(19) United States
(12) Patent Application Publication
(10) Pub. No.: US 2023/0104281 A1
(43) Pub. Date: Apr. 6, 2023

(54) FLUORESCENT RHODAMINE DYES WITH ENHANCED CELL PERMEABILITY

(71) Applicant: Max-Planck-Gesellschaft zur Foerderung der Wissenschaften e. V., Muenchen (DE)

(72) Inventors: Jonas BUCEVICIUS, Goettingen (DE); Ruta GERASIMAITE, Goettingen (DE); Georgij KOSTIUK, Goettingen (DE); Grazvydas LUKINAVICIUS, Goettingen (DE)

(21) Appl. No.: 17/798,440

(22) PCT Filed: Feb. 11, 2021
(86) PCT No.: PCT/EP2021/053319

§ 371 Date: Aug. 9, 2022

(30) Foreign Application Priority Data
Feb. 12, 2020 (EP) 20156964.7

(51) Int. Cl. G01N 33/58; C09B 11/24; G01N 21/64

(52) U.S. Cl. CPC G01N 33/582; C09B 11/24; G01N 21/6428; G01N 2021/6439

(57) ABSTRACT

The invention relates to novel fluorescent rhodamine dyes with enhanced cell permeability which are rhodamine 4′-isomers having the following general structural formula A: wherein Z is selected from O(alkyl), O(aryl), S(aryl), S(O)(alkyl), S(O) (aryl), S(O)₂(alkyl), S(O)₂(aryl), S(O)₂(—O-alkyl), S(O)₂(—O-aryl), S(O)₂NH(alkyl), S(O)₂NH(aryl), S(O)₂N(alkyl)₂, S(O)₂N(aryl)₂, S(O)₂N(alkyl)(aryl), C(O)O(alkyl), C(O)O(aryl), C(O)(alkyl), C(O)(aryl), P(O)OH(—NH-alkyl), P(O)OH(—O-alkyl), P(O)OH(—NH-aryl), P(O)OH(—O-aryl), P(O)(—O-alkyl)₂, P(O)(—NH-alkyl)₂, P(O)OH(—N(alkyl)₂), P(O)OH(—N(aryl)₂), P(O)(—N(aryl)₂)₂, P(O) (—N(alkyl)₂)(—N(alkyl)₂), P(O)(—O-aryl)₂, P(O)(—NH-aryl)₂, P(O)(—O-alkyl)(-O-aryl), P(O)(—NH-alkyl)(-O-aryl), P(O)(-O-alkyl)(-NH-aryl), P(O)(—NH-alkyl)(-NH-aryl), C(O)OH, C(O)NH(alkyl), C(O)NH(aryl), CON(alkyl)₂, CON(aryl)₂, in particular C(O)OH, C(O)NH(alkyl), C(O)NH(aryl), CON(alkyl)₂, CON(aryl)₂, C(O)O(alkyl) and C(O)O(aryl), or any group which is neither Cl, NH₂ or NO₂ and which induces a neighboring group effect via steric, ionic or bonding interactions with the adjacent carboxyl group resulting in a shift of the equilibrium between zwitterionic form and spirolactone form towards the spirolactone form. The invention further relates to 4′-isomer derivatives and probes comprising such 4′-isomers coupled to at least one reactive group or ligand which is capable to interact with or bind to other molecules, wherein said reactive group or ligand may be coupled to the rhodamine 4′-isomer fluorophore either directly or via a linker. Another aspect of the invention relates to the use of these compounds and conjugates as labels in microscopic, spectroscopic and other imaging techniques and/or as cell permeable substances penetrating through membranes of living and fixed cells in vivo or in vitro.

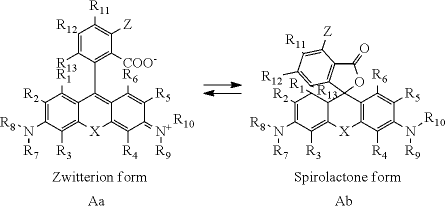

### BACKGROUND OF THE INVENTION

Modern super-resolution fluorescence microscopy techniques permit observation of biological processes in living organisms down to molecular level (Sahl, S. J.; Hell, S. W.; Jakobs, S., Nat. Rev. Mol. Cell. Biol. 2017, 18 (11), 685-701; Hell, S. W, Angew Chem Int Ed Engl 2015, 54 (28), 8054-66). Success of imaging experiments depends on the availability of biocompatible fluorescent probes for the specific labelling of cellular structures. Usually, these probes are constructed by coupling a fluorescent dye to a small molecule ligand via a linker to target a protein of interest. A linker and a fluorophore make significant contributions to the final properties of such probes often resulting in compromised cell permeability and off-targeting (Wang, L.; Frei, M. S.; Salim, A.; Johnsson, K., J Am Chem Soc 2019, 141 (7), 2770-2781). The ideal biocompatible fluorophore fulfil at least the following five criteria: First, the fluorescent dye should be highly photostable avoiding rapid bleaching in the course of an experiment. Second, it should have high extinction coefficient and quantum yield that results in a good brightness. Third, a derivatization of the fluorescent dye molecule with (i) ligands that specifically bind to other (bio)molecules in vitro or in vivo or, (ii) reactive groups such as activated esters (iii) molecules that can control the fluorescence properties of the fluorophore should be possible. Fourth, it should not interact with biomolecules present in the cell and show minimal background. Fifth, the fluorophore should be membrane permeable. Numerous fluorophores exist that fulfil the first three criteria, the tetramethylrhodamine (TMR) being an example of fluorophore whose derivatives show relatively low permeability and high off-targeting. Only few fluorophores available that also fulfil the fifth criterion. Recently several groups introduced fluorophores that show minimal background binding or are fluorogenic and are able to mask this unspecific binding. These fluorophores are various derivatives of rhodamines. The most prominent examples are carbopyronines (CP) (Butkevich A. N.; Mitronova G. Y.; Sidenstein. S. C.; Klocke J. L.; Kamin D.; Meineke D. N. H.; D'Este E.; Kraemer P. T.; Danzl J. G.; Belov V. N.; Hell S. W., Angew. Chem. Int. Ed. 2016, 55, 3290-3294) and silicon-rhodamines (SiR) (Figure 1) (Lukinavic̆ius G; Umezawa K; Olivier N; Honigmann A; Yang G; Plass T; Mueller V; Reymond L; Correa IR Jr; Luo ZG; Schultz C; Lemke EA; Heppenstall P; Eggeling C; Manley S; Johnsson K, Nat Chem, 2013, 5(2), 132-139).

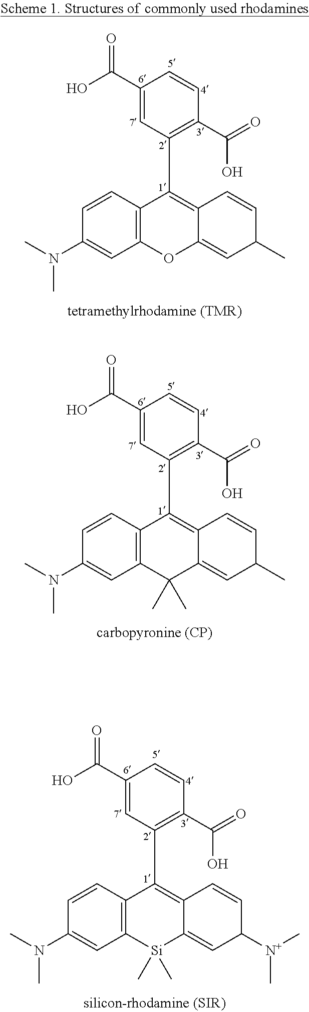

A lot of interest is dedicated to the rhodamine class fluorophores, which are able to cycle between non-fluorescent spirolactone and a fluorescent zwitterion forms (Scheme 2). This property is exploited to generate fluorogenic probes, which are sensitive to number of environmental factors: pH, ion concentration, enzyme, tension force, local microenvironment polarity or light (Uno, S. N.; Tiwari, D. K.; Kamiya, M.; Arai, Y.; Nagai, T.; Urano, Y., Microscopy (Oxf) 2015, 64 (4), 263-77). Because spirolactone is a more hydrophobic molecule than zwitterion, the change of the equilibrium between these two forms contributes to the permeability of the rhodamine-based fluorescent probes (Lukinavic̆ius, G.; Reymond, L.; Umezawa, K.; Sallin, O.; D'Este, E.; Gottfert, F.; Ta, H.; Hell, S. W.; Urano, Y.; Johnsson, K., J. Am. Chem. Soc. 2016, 138 (30), 9365-8.

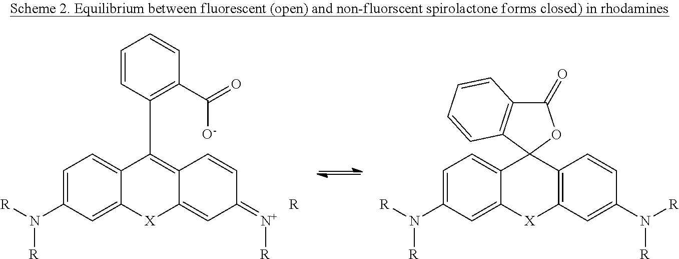

It is assumed that regular rhodamines and carbon-substituted analogs (carbopyronines) have this equilibrium shifted towards zwitterion form, resulting in relatively poor permeability. Several studies attempted to induce spirolactone form preference by introduction of the electron-withdrawing groups in the xanthene core (Zheng, Q.; Ayala, A. X.; Chung, I.; Weigel, A. V.; Ranjan, A.; Falco, N.; Grimm, J. B.; Tkachuk, A. N.; Wu, C.; Lippincott-Schwartz, J.; Singer, R. H.; Lavis, L. D., ACS Cent Sci 2019, 5 (9), 1602-1613) or in the benzoic acid substituent (Wang, L.; Tran, M.; D'Este, E.; Roberti, J.; Koch, B.; Xue, L.; Johnsson, K., bioRxiv 2019, 690867). However, all these approaches result in a bulkier core structure and alter physicochemical properties of the dyes.

The marking of proteins and nucleic acids with fluorescent dyes in living cells represents a widely used technique in life sciences and medical research. Multiple strategies have been developed to reach this goal. The most frequently used methods are: the tetracysteine tag that binds to biarsenical fluorophores, the SNAP/CLIP-tag that irreversibly reacts with benzylguanine (BG) or benzylcytosine (BC) derivatives (Keppler, A.; Gendreizig, S.; Gronemeyer, T.; Pick, H.; Vogel, H.; Johnsson, K., Nat Biotechnol 2003, 21 (1), 86-9), the Halo-tag that reacts with primary chlorides and dihydrofolate reductase that binds to trimethoprim derivatives (Hinner, M. J.; Johnsson, K., Curr Opin Biotechnol 2010, 21 (6), 766-76). Alternatively, a specific fluorescence labelling of a protein of interest can be achieved through the incorporation of an unnatural, fluorescent amino acid (Liu, C. C.; Schultz, P. G., Annu Rev Biochem 2010, 79, 413-44). Finally, proteins, DNA or RNA can be labelled using specifically binding ligands. The final probe is composed of the targeting moiety, linker and fluorescent dye. All these components contribute to the final properties of the probe. One on the most important property is cell membrane permeability, which allows efficient delivery of fluorophore to the target. This problem is often overcome by delivering fluorophore into the cell through invasive methods such as microinjection (Keppler, A.; Arrivoli, C.; Sironi, L.; Ellenberg, J., Biotechniques 2006, 41 (2), 167-70, 172, 174-5), bead-loading (Maurel, D.; Banala, S.; Laroche, T.; Johnsson, K., ACS Chem Biol 2010, 5 (5), 507-16) or electroporation (Jones, S. A.; Shim, S. H.; He, J.; Zhuang, X., Nat Methods 2011, 8 (6), 499-508).

Kocisova et al., in Journal of Molecular Structure, vol. 651-653 (2003), pages 115-122, disclose a carboxytetramethylrhodamine conjugate which has been prepared by using the commercial available 5-carboxytetramethylrhodamine N-hydroxysuccinimide ester (see page 117, left column, lines 2-5) and, thus, represents a 5-carboxytetramethylrhodamine conjugate.

Clerc et al., in Chem Bio Chem, vol. 10, no. 16, (2009), pages 2638-2643, disclose a rhodamine-tagged Syringolin A (SyIA) derivative which has been prepared by using a solution of a commercial available mixture of 5- and 6-rhodamine amine (see page 2642, left column, under synthesis of Rh-SyIA) and, thus, represents a 5/6-rhodamine conjugate.

Jolimaitre et al., in Chemistry and Physics of Lipids, vol. 133, no. 2 (2005), pages 215-223, disclose a tetramethylcarboxyrhodamine conjugate which has been prepared by reacting the N-amine of Lysine with the activated hydroxybenzotriazolyl ester of 5,6-tetramethylcarboxyrhodamine (see page 218, right column, third paragraph) and, thus, represents a 5,6-tetramethylcarboxyrhodamine conjugate.

Gee et al., in Bioorganic & Medicinal Chemistry Letters, vol. 11, no. 16, (2001), pages 2181-2183, disclose a caged Q-rhodamine dextran conjugate which has been prepared by using bisacylated 5- and 6-carboxy-Q-rhodamine (see page 2181, right column, second paragraph) and, thus, represents a mixture of 5/6-carboxyrhodamine isomers.

However, such 5′- and 6′-isomers of rhodamine are not able to provide a neighbouring group effect like the rhodamine 4′ isomers of the present invention.

In view of the drawbacks of fluorescent dyes of the prior art and the alternative strategies to obviate the limitations of their delivery to living cells and tissues, the main object of the present invention is to provide improved fluorescent dyes, in particular fluorescent dyes with enhanced cell permeability while still exhibiting desirable optical characteristics such as high photostability, high extinction coefficients and high quantum yields.

This objective has been achieved by providing novel compounds and fluorescent dyes which are rhodamine 4′ isomers according to claims 1-11, conjugates comprising said compounds and dyes according to claim 12, as well as the applications of the claimed compounds according to claims 13 to 22.

### DESCRIPTION OF THE INVENTION

The novel fluorescent dyes of the invention are rhodamine 4′-isomers that have the following general structural formula A

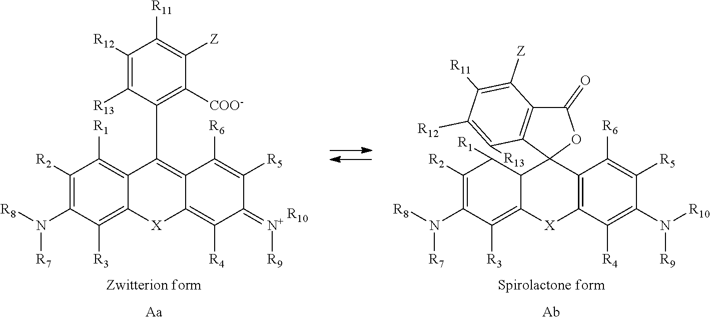

wherein
R₁, R₂, R₃, R₄ R₅ R₆ R₇ R₈ R₉ R₁₀ R₁₁ R₁₂ R₁₃ are independently selected from H, halogen, D, CN, OH, O(alkyl), O(aryl), SH, S(alkyl), S(aryl), S(O)(alkyl), S(O)(aryl), S(O)₂(alkyl), S(O)₂(aryl), N₃, NH₂, NH(alkyl), N(alkyl)₂, NH(aryl), NH(aryl)₂, NO₂, CHO, C(O)(alkyl), C(O)(aryl), COOH, COO(alkyl) COO(aryl), C(O)NH(alkyl), C(O)NH(aryl), C(O)N(alkyl)₂, C(O)N(aryl)₂, P(O)OH(alkyl), P(O)OH(aryl), P(O)(—O-alkyl)₂, P(O)(—O-aryl)₂, PO₃H₂, SO₃H, alkyl, substituted alkyl, alkenyl, substituted alkenyl;
R₂ and R₈ or R₇ and R₈ taken together may form a cyclic structure;
R₅ and R₉ or R₉ and R₁₀ taken together may form a cyclic structure;
R₃ and R₇ or R₄ and R₉ taken together may form a cyclic structure;
R₁ and R₂ or R₅ and R₆ taken together may form a cyclic structure;
X is selected from CR₁₄R₁₅, a heteroatom, in particular O, S, NR₁₄, SO₂, P(O)OH, P(O)OR₁₄, SiR₁₄R₁₅, GeR₁₄R₁₅, where R₁₄ and R₁₅ are alkyl or aryl;
Z is selected from O(alkyl), O(aryl), S(aryl), S(O)(alkyl), S(O)(aryl), S(O)₂(alkyl), S(O)₂(aryl), S(O)₂(—O-alkyl), S(O)₂(—O-aryl), S(O)₂NH(alkyl), S(O)₂NH(aryl), S(O)₂N(alkyl)₂, S(O)₂N(aryl)₂, S(O)₂N(alkyl)(aryl),, C(O)O(alkyl), C(O)O(aryl), C(O)(alkyl), C(O)(aryl), P(O)OH(—NH-alkyl), P(O)OH(—O-alkyl), P(O)OH(—NH-aryl), P(O)OH(—O-aryl), P(O)(—O-alkyl)₂, P(O)(—NH-alkyl)₂, P(O)OH(—N(alkyl)₂), P(O)OH(—N(aryl)₂), P(O)(—N(aryl)₂)₂, P(O)(—N(alkyl)₂)(—N(alkyl)₂), P(O)(—O-aryl)₂, P(O)(—NH-aryl)₂, P(O)(—O-alkyl)(-O-aryl), P(O)(—NH-alkyl)(-O-aryl), P(O)(—O-alkyl)(-NH-aryl), P(O)(—NH-alkyl)(-NH-aryl), C(O)OH, C(O)NH(alkyl), C(O)NH(aryl), CON(alkyl)₂, in particular C(O)OH, C(O)NH(alkyl), C(O)NH(aryl), CON(alkyl)₂, CON(aryl)₂, C(O)O(alkyl) and C(O)O(aryl), or any group which induces a neighboring group effect via steric, ionic or bonding interactions with the adjacent carboxyl group involved in spirolactone formation resulting in a shift of the equilibrium between zwitterionic form and spirolactone form towards the spirolactone form as indicated by an increased D₅₀ value of the 4′-isomer compared to the D₅₀ value of a reference isomer, in particular a 5′- or 6′-isomer, and preferably an absolute D₅₀ value of the 4′-isomer which is a numeric value of at least 13 or 14, more preferred at least 15, 20, or 25, wherein said D₅₀ value represents the dielectric constant at which the absorbance of a dye sample in a mixture of 1,4-dioxane and water is halved.

The D₅₀ values indicated herein are determined by measuring the respective absorbance of a sample (dye isomer or derivative or conjugate thereof), in varied water-1,4-dioxane mixtures with known dielectric constants according to an established method (e.g. Åkerlöf G, Short AO. J Am Chem Soc 1936, 58(7): 1241-1243). D₅₀ is commonly used as numeric value for the evaluation of the equilibrium changes and represents a dielectric constant at which absorbance is halved (Butkevich A. N.; Mitronova G. Y.; Sidenstein. S. C.; Klocke J. L.; Kamin D.; Meineke D. N. H.; D'Este E.; Kraemer P. T.; Danzl J. G.; Belov V. N.; Hell S. W., Angew. Chem. Int. Ed. 2016, 55, 3290-3294).

The D₅₀ values were calculated using the following equation:

$$A = {A}_{0} + ( {A}_{m a x} - {A}_{0} ) / ( 1 + {( \frac{{D}_{50}}{d} )}^{Hill} ) \qquad ( 1 )$$

where A₀—absorbance at λₘₐₓ at εᵣ=0, λₘₐₓ—the highest reached absorbance at λₘₐₓ, d—dielectric constant of 1,4-dioxane-water mixture at a given point, Hill—Hill slope coefficient determining the steepness of a dose-response curve, D₅₀—corresponds to d value that provokes half of the absorbance amplitude (λₘₐₓ−A₀).

The above indicated absolute D₅₀ value of 15 as obtained by said method corresponds to a mixture of 75% 1,4-dioxane and 25% water.

Preferably R₁ to R₁₃ are each independently selected from H, alkyl, in particular C₁-C₂₀ alkyl, C₁-C₁₂ alkyl, or C₁-C₆ alkyl.

X is preferably selected from O, CR₁₄R₁₅, SiR₁₄R₁₅, GeR₁₄R₁₅ where R₁₄ and R₁₅ are alkyl, in particular C₁-C₂₀ alkyl, C₁-C₁₂ alkyl, or C₁-C₆ alkyl, or aryl, in particular phenyl or naphthyl.

Said group Z which induces a neighboring group effect via steric, ionic or bonding interactions according to the invention is neither Cl, NH₂ or NO₂ and is preferably selected from the group consisting of C(O)OH, C(O)NH(alkyl), C(O)NH(aryl), CON(alkyl)₂, CON(aryl)₂, C(O)O(alkyl) and C(O)O(aryl), with alkyl in particular C₁-C₂₀ alkyl, C₁-C₁₂ alkyl, or C₁-C₆ alkyl.

In some specific embodiments, the fluorescent dyes according to the present invention have one of the following structural formulae B-E

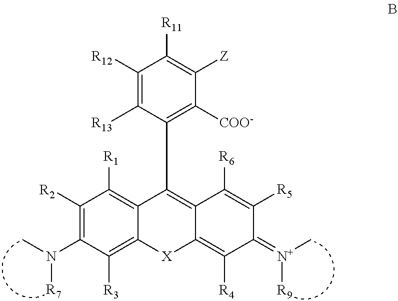

wherein the cyclic structure formed by R₇ and R₈ and/or by R₉ and R₁₀ taken together represents a non-aromatic heterocycle, in particular selected from azetidine, pyrrolidine, piperidine, morpholine, azepane or azecane;

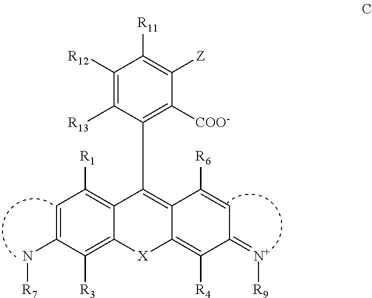

wherein the cyclic structure formed by R₂ and R₈ and/or by R₅ and R₁₀ taken together represents an aromatic or non-aromatic 5 or 6 atom membered heterocyclic structure, in particular selected from azetidine, pyrrolidine, piperidine, azepane or azecane;

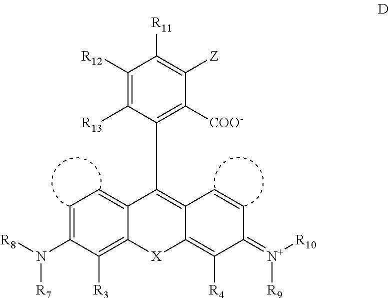

wherein the cyclic structure formed by R₁ and R₂ and/or by R₅ and R₆ taken together represents an aromatic or non-aromatic 5 or 6 atom membered heterocyclic structure, in particular selected from azetidine, pyrrolidine, piperidine, azepane or azecane;

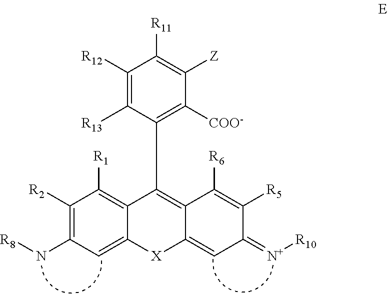

wherein the cyclic structure formed by R₃ and R₇ and/or by R₄ and R₉ taken together represents an aromatic or non-aromatic 5 or 6 atom membered heterocyclic structure, in particular selected from azetidine, pyrrolidine, piperidine, azepane or azecane.

In other specific embodiments, the fluorescent dyes according to the present invention have one of the following structural formulae

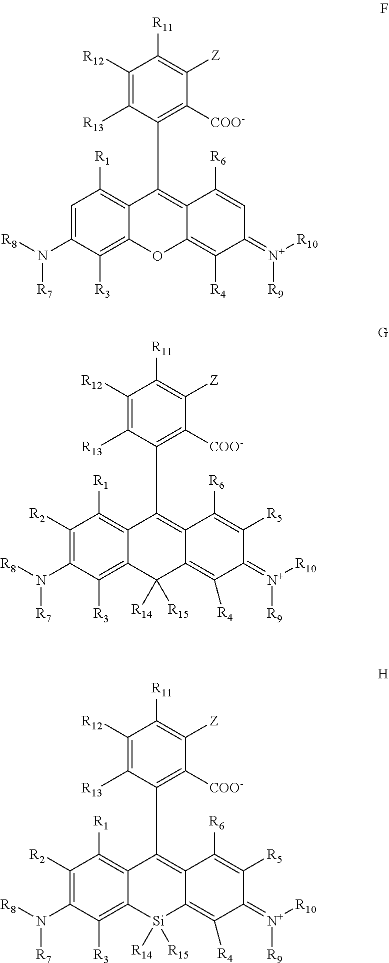

wherein
R₁, R₂, R₃, R₄ R₅ R₆ R₇ R₈ R₉ R₁₀ R₁₁ R₁₂ R₁₃ are independently selected from H, halogen, D, CN, OH, O(alkyl), O(aryl), SH, S(alkyl), S(aryl), S(O)(alkyl), S(O)(aryl), S(O)₂(alkyl), S(O)₂(aryl), N₃, NH₂, NH(alkyl), N(alkyl)₂, NH(aryl), NH(aryl)₂, NO₂, CHO, C(O)(alkyl), C(O)(aryl), COOH, COO(alkyl) COO(aryl), C(O)NH(alkyl), C(O)NH(aryl), C(O)N(alkyl)₂, C(O)N(aryl)₂, P(O)OH(alkyl), P(O)OH(aryl), P(O)(—O-alkyl)₂, P(O)(—O-aryl)₂, PO₃H₂, SO₃H, alkyl, bulky alkyl, substituted alkyl, alkenyl, substituted alkenyl, substituted bulky alkenyl;
R₂ and R₈ or R₇ and R₈ taken together may form a cyclic structure;
R₅ and R₉ or R₉ and R₁₀ taken together may form a cyclic structure;
R₃ and R₇ or R₄ and R₉ taken together may form a cyclic structure;
R₁ and R₂ or R₅ and R₆ taken together may form a cyclic structure;
Z is selected from O(alkyl), O(aryl), S(aryl), S(O)(alkyl), S(O)(aryl), S(O)₂(alkyl), S(O)₂(aryl), S(O)₂(—O-alkyl), S(O)₂(—O-aryl), S(O)₂NH(alkyl), S(O)₂NH(aryl), S(O)₂N(alkyl)₂, S(O)₂N(aryl)₂, S(O)₂N(alkyl)(aryl), C(O)O(alkyl), C(O)O(aryl), C(O)(alkyl), C(O)(aryl), P(O)OH(—NH-alkyl), P(O)OH(—O-alkyl), P(O)OH(—NH-aryl), P(O)OH(—O-aryl), P(O)(—O-alkyl)₂, P(O)(—NH-alkyl)₂, P(O)OH(—N(alkyl)₂), P(O)OH(—N(aryl)₂), P(O)(—N(aryl)₂)₂, P(O)(—N(alkyl)₂)(—N(alkyl)₂), P(O)(—O-aryl)₂, P(O)(—NH-aryl)₂, P(O)(—O-alkyl)(-O-aryl), P(O)(—NH-alkyl)(-O-aryl), P(O)(—O-alkyl)(-NH-aryl), P(O)(—NH-alkyl)(-NH-aryl), C(O)OH, C(O)NH(alkyl), C(O)NH(aryl), CON(alkyl)₂, in particular C(O)OH, C(O)NH(alkyl), C(O)NH(aryl), CON(alkyl)₂, CON(aryl)₂, C(O)O(alkyl) and C(O)O(aryl), or any group which induces a neighboring group effect via steric, ionic or bonding interactions as defined above.

More specifically, the fluorescent dyes have one of the following structural formulae:

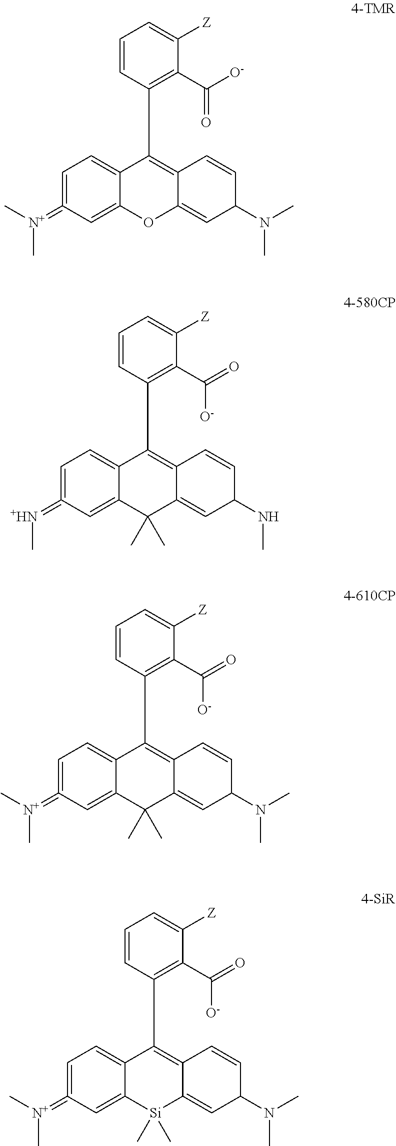

wherein
Z is selected from O(alkyl), O(aryl), S(aryl), S(O)(alkyl), S(O)(aryl), S(O)₂(alkyl), S(O)₂(aryl), S(O)₂(—O-alkyl), S(O)₂(—O-aryl), S(O)₂NH(alkyl), S(O)₂NH(aryl), S(O)₂N(alkyl)₂, S(O)₂N(aryl)₂, S(O)₂N(alkyl)(aryl), C(O)O(alkyl), C(O)O(aryl), C(O)(alkyl), C(O)(aryl), P(O)OH(—NH-alkyl), P(O)OH(—O-alkyl), P(O)OH(—NH-aryl), P(O)OH(—O-aryl), P(O)(—O-alkyl)₂, P(O)(—NH-alkyl)₂, P(O)OH(—N(alkyl)₂), P(O)OH(—N(aryl)₂), P(O)(—N(aryl)₂)₂, P(O)(—N(alkyl)₂)(—N(alkyl)₂), P(O)(—O-aryl)₂, P(O)(—NH-aryl)₂, P(O)(—O-alkyl)(-O-aryl), P(O)(—NH-alkyl)(-O-aryl), P(O)(—O-alkyl)(-NH-aryl), P(O)(—NH-alkyl)(-NH-aryl), C(O)OH, C(O)NH(alkyl), C(O)NH(aryl), CON(alkyl)₂, in particular C(O)OH, C(O)NH(alkyl), C(O)NH(aryl), CON(alkyl)₂, CON(aryl)₂, C(O)O(alkyl) and C(O)O(aryl), or any group which induces a neighboring group effect via steric, ionic or bonding interactions as defined above.

The terms “rhodamine 4′-isomer” or “4′-isomer”, are used herein as an abbreviated designation of the claimed rhodamine compounds which are, i.a., characterized by the presence of a substituent group Z at position 4′ which is located next to the benzene ring position carrying the COOH group which is involved in spirolactone formation and, thus, immediately adjacent to said COOH group.

The following scheme illustrates the numbering of positions as used in the present application.

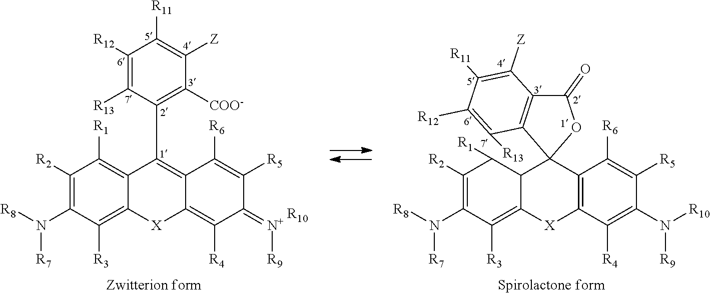

The term “substituted” as used herein, generally refers to the presence of one or more substituents, in particular substituents selected from the group comprising straight or branched alkyl, in particular C₁-C₄ alkyl, e.g. methyl, ethyl, propyl, butyl; isoalkyl, e.g. isopropyl, isobutyl (2-methylpropyl); secondary alkyl group, e.g. sec-butyl (but-2-yl); tert-alkyl group, e.g. tert-butyl (2-methylpropyl). Additionally, the term “substituted” may refer here to alkyl groups having at least one deuterium-, fluoro-, chloro- or bromo substituent instead of hydrogen atoms, or methoxy, ethoxy, 2-(alkyloxy)ethyloxy groups (alk-OCH₂CH₂O), and, in a more general case, oligo(ethylenglycol) residues of the art alk(OCH₂CH₂)ₙOCH₂CH₂—, where alk=CH₃, C₂H₅, C₃H₇, C₄H₁₀, and n=1-12.

The term “alkyl” refers to any alkyl group selected from the group comprising straight or branched alkyl, more specifically C₁-C₂₀ alkyl, C₁-C₁₂ alkyl, or C₁-C₆ alkyl, e.g. methyl, ethyl, propyl, butyl; isoalkyl, e.g. isopropyl, isobutyl (2-methylpropyl); secondary alkyl group, e.g. sec-butyl (but-2-yl); tert-alkyl group, e.g. tert-butyl (2-methylpropyl) etc. The alkyl group may be a bulky alkyl group which comprises, e.g., a large and/or highly branched alkyl group such as a tert.-alkyl group.

The term “alkenyl” refers to any alkenyl group selected from the group comprising straight or branched alkenyl, more specifically C₁-C₂₀ alkenyl, C₁-C₁₂ alkenyl, or C₁-C₆ alkenyl, e.g. an alkenyl group corresponding to one of the exemplary alkyl groups mentioned above. The alkenyl group may be a bulky alkylenyl group which comprises, e.g., a large and/or highly branched alkenyl group such as a tert.-alkenyl group.

The terms “aryl” or “aromatic group”, as used herein, generally refer to an unsubstituted or substituted mono-, bi- or tricyclic carbocyclic ring system having one, two or three aromatic rings. Representative but not limiting examples are including but not limited to phenyl, naphthyl, anthryl, azulyl, tetrahydronaphthyl, indanyl and indenyl.

The terms “aromatic heterocyclic group” or “heteroaromatic group”, as used herein, generally refer to an unsubstituted or substituted cyclic aromatic radical (residue) having from 5 to 10 ring atoms of which at least one ring atom is selected from S, O and N; the radical being joined to the rest of the molecule via any of the ring atoms. Representative, but not limiting examples are furyl, thienyl, pyridinyl, pyrazinyl, pyrimidinyl, pyrrolyl, imidazolyl, thiazolyl, oxazolyl, isooxazolyl, thiadiazolyl, oxadiazolyl, quinolinyl and isoquinolinyl.

The term “activated ester”, as used herein, generally refers to a carboxyl group substituted with a group which renders it more reactive with nucleophiles such as, but not limited to, free amino groups of peptides, polyaminoacids, polysaccharides, or analytes under such conditions that no interfering side reactions with other reactive groups of the nucleophile-carrying substance can usefully occur. Examples of such substituent groups that form activated esters directly or by introduction of the linker include N-succinimidyl, sulfo-N-succinimidyl, 1-benzotriazolyl, and the like.

The term “a positional isomer”, as used herein, generally refers to regioisomers of the discussed structures where a position of a functional group or other substituent on a parent structure changes, leading to different bonding patterns. The chemical composition and elemental analysis results of positional isomers are identical.

The term “linker”, as used herein, generally refers to a straight or branched alkyl chain which may be substituted by one or more functional groups and may include heteroatoms and/or aromatic groups and is connecting a targeting moiety (i.e. a reactive group or ligand) and fluorescent dye. More specifically, the linker may comprise or represent one or more of the following groups: Cₙ₊₁ alkyl linear or branched, e.g. methyl, ethyl, propyl, butyl; isoalkyl, e.g. isopropyl, isobutyl (2-methylpropyl); secondary alkyl group, e.g. sec-butyl (but-2-yl); tert-alkyl group, e.g. tert-butyl (2-methylpropyl), hydroxylated alkyl —(CH₂O)ₙ-linear or branched, fluorinated alkyl —(CHF)ₙ₊₁— or —(CF₂)ₙ₊₁—, alkenyl —(CH₂)ₖ—(CH═CH)ₙ₊₁—(CH₂)ₘ— with single or multiple double bonds, alkinyl —(CH₂)ₖ—(C≡C)ₙ₊₁—(CH₂)ₘ— with single or multiple triple bonds, tris(hydroxymethyl)aminomethane, O(alkyl)₂, O(aryl)₂, S(alkyl)₂, S(aryl)₂, S(O)(alkyl)₂, S(O)(aryl)₂, S(O)₂(alkyl)₂, S(O)₂(aryl)₂, N(alkyl)₂, NH(aryl)₂, C(O)(alkyl)₂, C(O)(aryl)₂, P(O)(—O-alkyl)₂, P(O)(—O-aryl)₂, C(O)NH(alkyl)₂. Here k, n and m=0-20.

The present invention further relates to rhodamine 4′-isomer derivatives or probes comprising a rhodamine 4′-isomer having one of the general formulae A-H as described above, wherein Z is selected from O(alkyl), O(aryl), S(aryl), S(O)(alkyl), S(O)(aryl), S(O)₂(alkyl), S(O)₂(aryl), S(O)₂(—O-alkyl), S(O)₂(—O-aryl), S(O)₂NH(alkyl), S(O)₂NH(aryl), S(O)₂N(alkyl)₂, S(O)₂N(aryl)₂, S(O)₂N(alkyl)(aryl), C(O)O(alkyl), C(O)O(aryl), C(O)(alkyl), C(O)(aryl), P(O)OH(—NH-alkyl), P(O)OH(—O-alkyl), P(O)OH(—NH-aryl), P(O)OH(—O-aryl), P(O)(—O-alkyl)₂, P(O)(—NH-alkyl)₂, P(O)OH(—N(alkyl)₂), P(O)OH(—N(aryl)₂), P(O)(—N(aryl)₂)₂, P(O)(—N(alkyl)₂)(—N(alkyl)₂), P(O)(—O-aryl)₂, P(O)(—NH-aryl)₂, P(O)(—O-alkyl)(-O-aryl), P(O)(—NH-alkyl)(-O-aryl), P(O)(—O-alkyl)(-NH-aryl), P(O)(—NH-alkyl)(-NH-aryl), in particular C(O)OH, C(O)NH(alkyl), C(O)NH(aryl), CON(alkyl)₂, or any group which induces a neighboring group effect via steric, ionic or bonding interactions as defined above, which rhodamine 4′-isomer is coupled to at least one reactive group or ligand which is capable to interact with or bind to other molecules. Said reactive group or ligand may be coupled to the rhodamine 4′-isomer fluorophore either directly or via a linker as defined above.

More specifically, Z is selected from the group consisting of C(O)OH, C(O)NH(alkyl), C(O)NH(aryl), CON(alkyl)₂, CON(aryl)₂, C(O)O(alkyl) and C(O)O(aryl).

Typically, the linker comprises or consists of a straight or branched alkyl chain with 1-21 C atoms which may be substituted by one or more functional groups and may include heteroatoms and/or aromatic groups, and preferably represents a moiety selected from the following group:

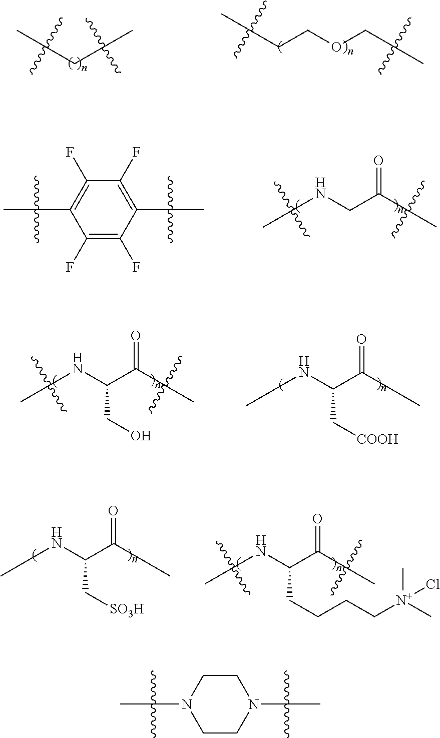

with n=an integer of 1-20.

More specifically, said reactive group may be selected from the group comprising an activated ester, an amine, a thiol, an azide, an ethyne, a maleimide, a tetrazine, N-hydroxysuccinimide or an alcohol group, or wherein the ligand is a ligand which binds specifically to a protein, peptide, nucleotide or nucleic acid, carbohydrate, iodoacetamide or which is capable to effect or participate in chelation of NH₄⁺ or metal ions, in particular Li⁺, Na⁺, K⁺, Cs⁺, Rb⁺, Cu⁺, Tl⁺, Hg⁺, Ag⁺, Au⁺, Ca²⁺, Ba²⁺, Sr²⁺, Be²⁺, Zn²⁺, Ni²⁺, Cu²⁺, Mg²⁺, Co²⁺, Fe²⁺, Mn²⁺, Pt²⁺, Cd²⁺, Hg²⁺, Sn²⁺, Pb²⁺, Au³⁺ Cr³⁺, Co³⁺, Fe³⁺, Al³⁺, Ga³⁺, In³⁺, Sc³⁺, Ti³⁺, Tl³⁺, V³⁺, Y³⁺, La³⁺, Pt⁴⁺, Pb⁴⁺, Ce⁴⁺, Ge⁴⁺, Th⁴⁺, Zr⁴⁺, U⁴⁺.

In a specific embodiment of the rhodamine 4′-isomer derivative according to the present invention, the reactive group or ligand is coupled directly or via a linker to group Z.

In this case, a linker may be also provided by Z, if said group Z as defined above already comprises a linking group, in particular as defined above.

In a preferred embodiment thereof, the reactive group or ligand is coupled by an amide bond formed between a carboxyl functional group or amine functional group provided by Z and an amine functional group or carboxyl functional group provided by the linker or ligand.

However, in other specific embodiments, the reactive group or ligand may be also coupled to other coupling sites on the same ring or other rings of the rhodamine structure, in particular at positions 6′ or 5′, preferably at position 6′.

In some other specific embodiments, the ligand of the rhodamine 4′-isomer derivative which is capable to interact with or bind to other molecules is benzylguanine and the protein is SNAP-tag, or the ligand is benzylcytosine and the proteine is CLIP-tag, or the ligand is a primary alkyl chloride and the protein is Halo-tag, or the ligand is trimethoprim and the protein is dihydrofolate reductase.

The rhodamine 4′-isomer derivative or probe of the invention has an increased D₅₀ value compared to the D₅₀ value of a reference isomer derivative, in particular a 5′- or 6′ isomer derivative, which is typically higher than that of the corresponding 5′- or 6′-isomer derivative by a difference of at least 10.

Preferably, the absolute D₅₀ value of the 4′-isomer derivative or probe is a numeric value of at least 15, preferably at least 20 or 25, and generally is in the range from 15 to 85, typically in the range from 20 to 75 or from 25 to 70.

More specifically, if X═O in the above general formulae A-H, preferably the absolute D₅₀ value of the 4′-isomer derivative or probe is in the range from 20 to 30 or 20 to 35, if X═CR₁₄R₁₅, preferably the absolute D₅₀ value of the 4′-isomer derivative or probe is in the range from 55 to 70, if X═SiR₁₄R₁₅ or GeR₁₄R₁₅, preferably the absolute D₅₀ value of the 4′-isomer derivative or probe is in the range from 70 to 80 or 70 to 85.

A further aspect of the present invention relates to conjugates comprising a rhodamine 4′-isomer or rhodamine 4′-isomer derivative as described above which is coupled to or associated with a molecule selected from the group comprising a peptide, protein, in particular an enzyme or antibody, tubulin or actin, a nucleotide, nucleic acid sequence, including DNA and RNA, a lipid, carbohydrate, an organic or inorganic polyphosphate, a drug, in particular a pharmaceutical drug, or a metabolite, in particular a metabolite of said drug, NH₄⁺, a metal ion, in particular Li⁺, Na⁺, K⁺, Cs⁺, Rb⁺, Cu⁺, Tl⁺, Hg⁺, Ag⁺, Au⁺, Ca²⁺, Ba²⁺, Sr²⁺, Be²⁺, Zn²⁺, Ni²⁺, Cu²⁺, Mg²⁺, Co²⁺, Fe²⁺, Mn²⁺, Pt²⁺, Cd²⁺, Hg²⁺, Sn²⁺, Pb²⁺, Au³⁺ Cr³⁺, Co³⁺, Fe³⁺, Al³⁺, Ga³⁺, In³⁺, Sc³⁺, Ti³⁺, Tl³⁺, V³⁺, Y³⁺, La³⁺, Pt⁴⁺, Pb⁴⁺, Ce⁴⁺, Ge⁴⁺, Th⁴⁺, Zr⁴⁺, U⁴⁺, or complex thereof.

The conjugation may comprise formation of at least one covalent chemical bond or at least one molecular complex with a chemical entity or substance, such as amine, carboxylic acid, aldehyde, alcohol, aromatic compound, heterocycle, dye, amino acid, amino acid residue coupled to any chemical entity or molecule, in particular as listed above.

More specifically, the conjugation may comprise formation of at least one covalent chemical bond or at least one molecular complex with a chemical compound by a reaction or association with a functional group present in this compound such as amine, carboxylic acid, aldehyde, alcohol, aromatic compound, heterocycle, dye, amino acid, amino acid residue coupled to any chemical compound of interest, in particular as listed above.

The present invention provides a new class of rhodamine positional isomers, i.e. rhodamine 4′-isomers as defined above, and derivatives thereof that (i); possess high photostability (ii); have high extinction coefficients and high quantum yields (iii); can be derivatized with different molecules (iv); show little or no background binding to cellular biomolecules (v) are extremely membrane-permeable due to a neighboring group effect illustrated in the general structure of the new 4′-isomer dyes shown below

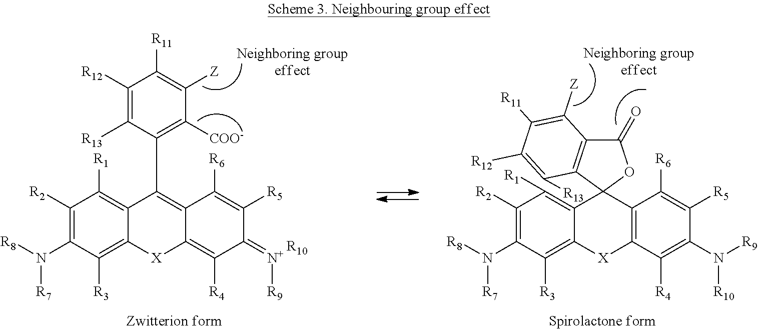

As mentioned, the essential feature of these novel fluorescent dyes is the presence of neighbouring groups Z in the benzene ring of the fluorophore. This provokes a specific effect, herein denoted as neighbouring group effect (NGE), which is a superposition of inductive, hydrogen bonding and steric effects (with the latter effects typically more relevant and a merely inductive effect typically will not be sufficient). For example, a significant NGE effect will be typically induced by groups Z which are bulky (steric interactions) and/or which are able to form hydrogen bonding with the carboxyl group responsible for spirolactonization. The exemplary groups Z listed above exhibit one or more of these properties.

This neighbouring group effect is only present in the 4′-isomers of rhodamines and influences the carboxyl group present in the benzene ring, changes spirolactonization rate and shifts equilibrium between spirolactone and zwitterion (see FIG. 1a). Said NGE effect can be detected by NMR or HPLC methods (FIG. 1): Rhodamine 4′-isomers containing NGE show significant NMR chemical shift of the interacting atoms which are not bound via covalent chemical bond and increased HPLC retention times. Most important the effect of NGE can be observed by measuring a change of D₅₀ value compared to D₅₀ value of other positional isomers and similarly with increased IC₅₀ values of conjugates. The NGE is especially profound if a linker or a ligand comprising an amine functional group is attached to a carboxylic group Z via formation of an amide bond resulting in conversion of the carboxylic group to carboxamide.

Generally, the NGE of 4′-isomer derivatives is higher than that of corresponding 4′-isomers as such because of the steric and electronic effects induced by substitutents.

Surprisingly, this neigbouring group effect results in dramatically increased cell membrane permeability while keeping all photophysical properties of the compounds almost unchanged.

### General Synthesis of the Novel Fluorescent Dyes, Probes and Conjugates of the Invention

### A. Synthesis of Rhodamine 4′-Isomer Dyes

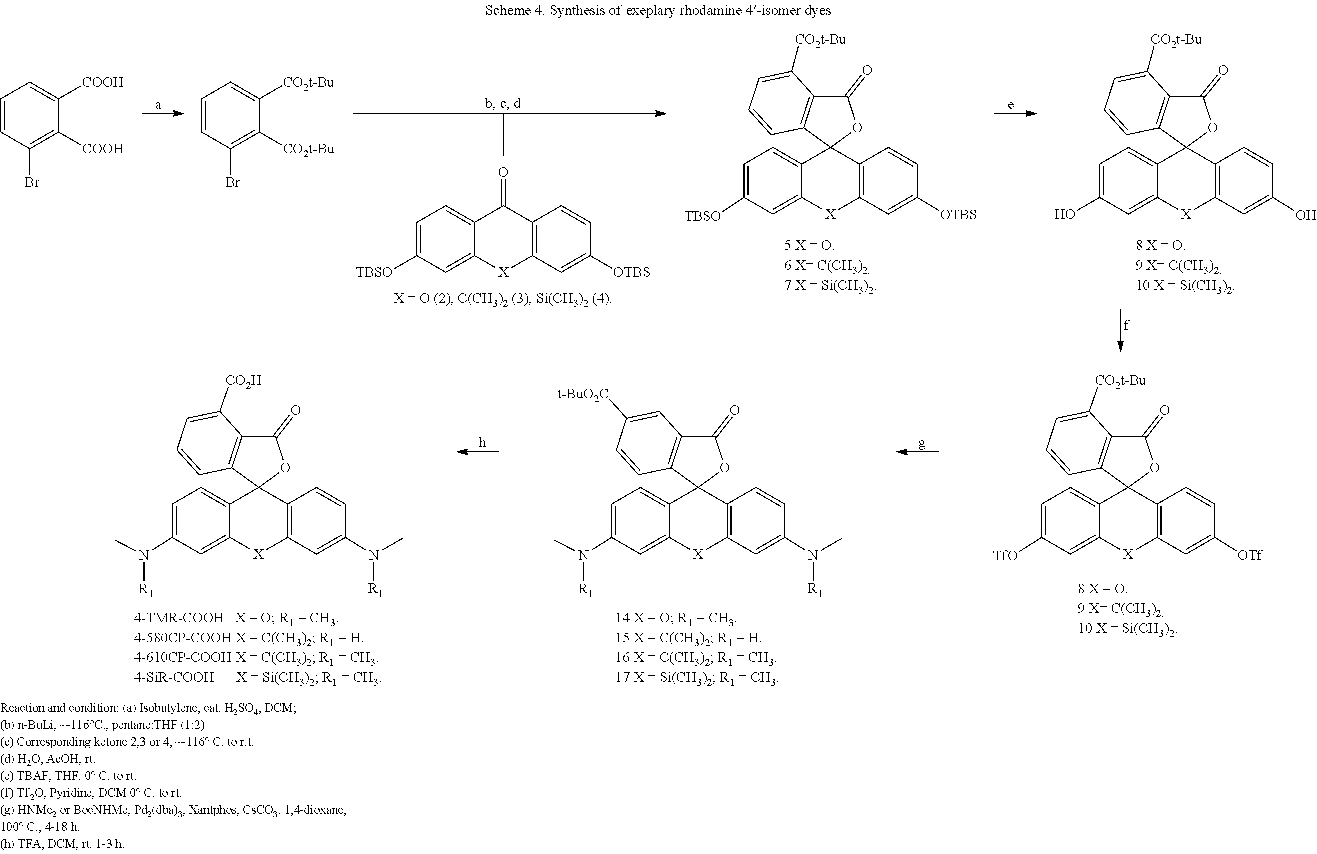

The general strategy for the synthesis of the novel rhodamine 4′ isomers is outlined above. More details with respect to the synthesis of exemplary compounds are provided in Synthesis Example A below.

### B. Synthesis of Rhodamine 4′-Isomer Fluorescent Probes

Generally, a fluorescent probe is comprised of the fluorophore molecule connected to the ligand, which interacts with the target of interest. The fluorescent probe absorbs light of a specific wavelength and emits light of a different, typically longer, wavelength and it acts as a marker of target of interest for analysis with fluorescent microscopy or spectroscopy. Fluorophore defines the operational wavelengths and ligand defines the target of interest. Fluorophore and the ligand is connected by covalent bond directly or through the linker. Optimal length and hydrophilicity of the linker is usually specific for each target of interest.

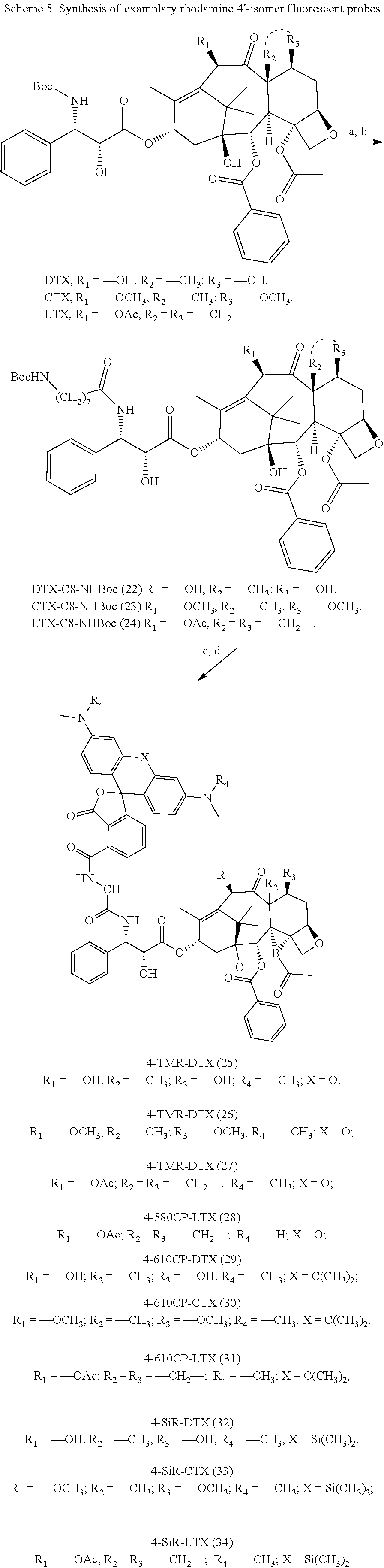

The general strategy for the synthesis of rhodamine 4′ isomer based fluorescent probes is outlined above. More details with respect to the synthesis of exemplary probes/conjugates are provided in Synthesis Example B below.

### Applications

In view of the advantageous properties of the novel fluorescent dyes according to the invention as outlined above, a further aspect of the present invention relates to their use as fluorescent dyes, in particular cell permeable fluorescent dyes, in a variety of applications.

Said fluorescent dyes may be a rhodamine 4′-isomer described above as such or a rhodamine 4′-isomer derivative comprising such a rhodamine 4′-isomer coupled to at least one reactive group or ligand which is capable to interact with or bind to other molecules. Said reactive group or ligand may be coupled to the rhodamine 4′-isomer fluorophore either directly or via a linker as defined above.

Said reactive group may be selected from the group comprising an activated ester, an amine, a thiol, an azide, an ethyne, a maleimide, a tetrazine, N-hydroxysuccinimide or an alcohol group, or wherein the ligand is a ligand which binds specifically to a protein, peptide, nucleotide or nucleic acid, carbohydrate, iodoacetamide or which is capable to effect or participate in chelation of NH₄⁺ or metal ions, in particular Li⁺, Na⁺, K⁺, Cs⁺, Rb⁺, Cu⁺, Tl⁺, Hg⁺, Ag⁺, Au⁺, Ca²⁺, Ba²⁺, Sr²⁺, Be²⁺, Zn²⁺, Ni²⁺, Cu²⁺, Mg²⁺, Co²⁺, Fe²⁺, Mn²⁺, Pt²⁺, Cd²⁺, Hg²⁺, Sn²⁺, Pb²⁺, Au³⁺ Cr³⁺, Co³⁺, Fe³⁺, Al³⁺, Ga³⁺, In³⁺, Sc³⁺, Ti³⁺, Tl³⁺, V³⁺, Y³⁺, La³⁺, Pt⁴⁺, Pb⁴⁺, Ce⁴⁺, Ge⁴⁺, Th⁴⁺, Zr⁴⁺, U⁴⁺.

In the following, a number of suitable or preferred ligands for various main targets are listed:

| Target | Ligand |
|---|---|
| Actin | 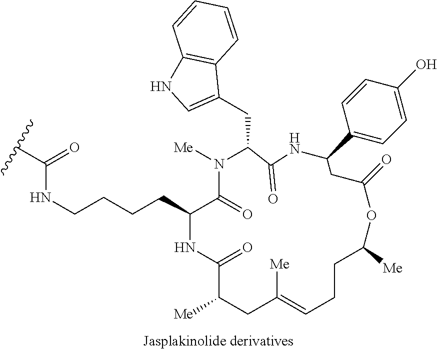 |
| | 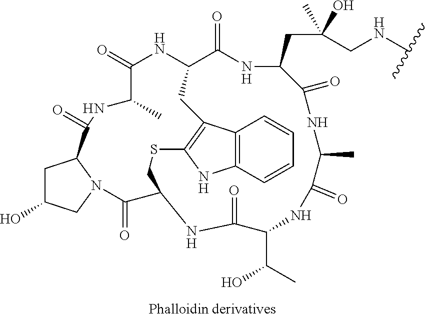 [SMILES: *NC[C@](C)(O)C[C@@H]1NC(=O)[C@@H]2Cc3c([nH]c4ccccc34)SC[C@@H](NC(=O)[C@@H]([C@H](C)O)NC(=O)[C@H](C)NC1=O)C(=O)N1C[C@H](O)C[C@H]1C(=O)N[C@@H](C)C(=O)N2] |
| Tubulin | 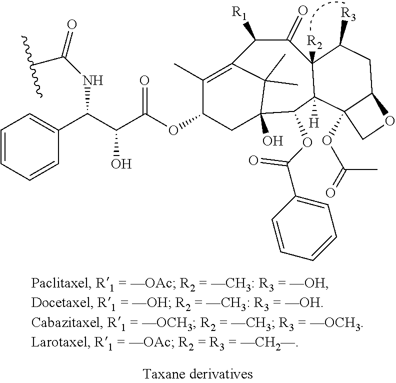 |
| | 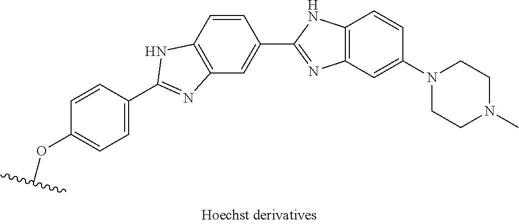 [SMILES: *Oc1ccc(-c2nc3cc(-c4nc5cc(N6CCN(C)CC6)ccc5[nH]4)ccc3[nH]2)cc1] |
| | 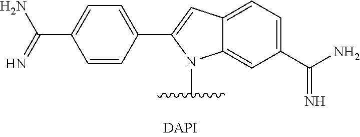 [SMILES: *n1c(-c2ccc(C(=N)N)cc2)cc2ccc(C(=N)N)cc21] |
| DNA | 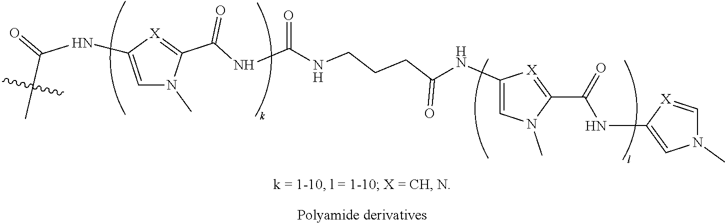 |
| | 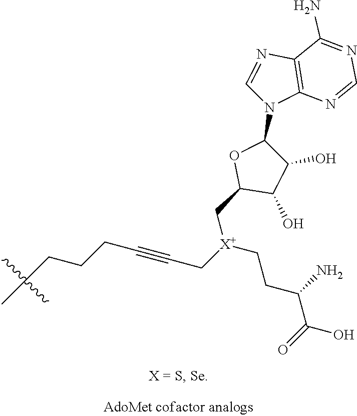 |
| Pepsin, cathepsins D and E. | 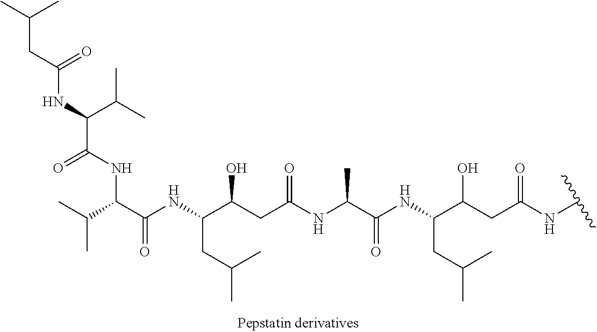 [SMILES: *NC(=O)CC(O)[C@H](CC(C)C)NC(=O)[C@H](C)NC(=O)C[C@H](O)[C@H](CC(C)C)NC(=O)[C@@H](NC(=O)[C@@H](NC(=O)CC(C)C)C(C)C)C(C)C] |
| Mitochondria | 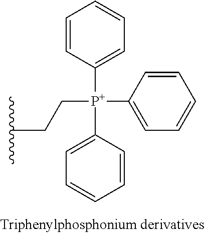 [SMILES: *CC[P+](c1ccccc1)(c1ccccc1)c1ccccc1] |
| FKBP12 | 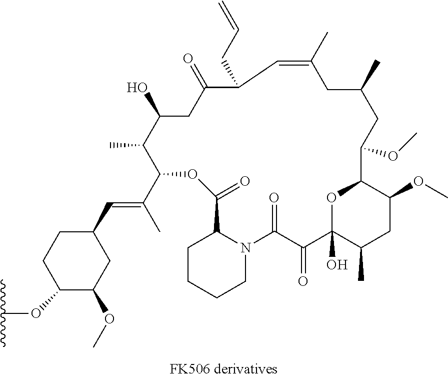 |
| Kinases | 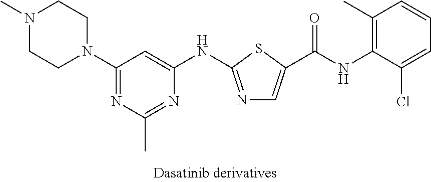 [SMILES: Cc1nc(Nc2ncc(C(=O)Nc3c(C)cccc3Cl)s2)cc(N2CCN(C)CC2)n1] |
| Halo-tag | 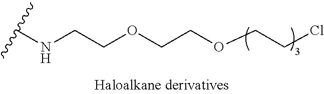 |
| SNAP-tag | 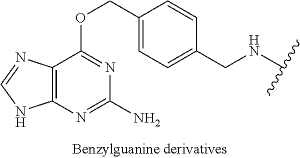 [SMILES: *NCc1ccc(COc2nc(N)nc3[nH]cnc23)cc1] |
| | 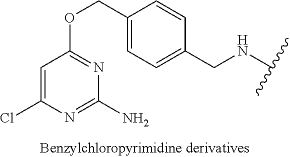 [SMILES: *NCc1ccc(COc2cc(Cl)nc(N)n2)cc1] |
| CLIP-tag | 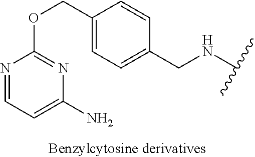 [SMILES: *NCc1ccc(COc2nccc(N)n2)cc1] |
| ACP tag and MCP-tag | 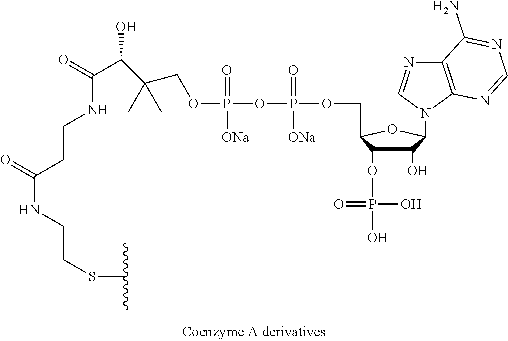 |
| His-tag | 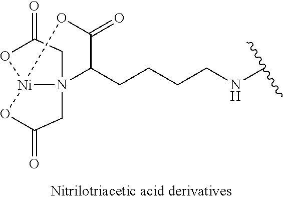 |
| Dihydrofolate reductase | 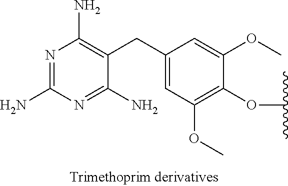 [SMILES: *Oc1c(OC)cc(Cc2c(N)nc(N)nc2N)cc1OC] |
| Pooly ADP ribose polymerase (PARP). | 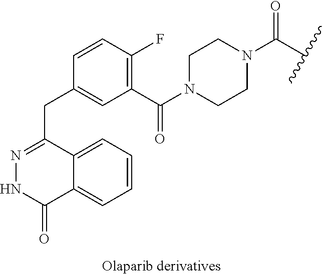 [SMILES: *C(=O)N1CCN(C(=O)c2cc(Cc3n[nH]c(=O)c4ccccc34)ccc2F)CC1] |
| | 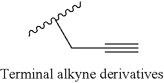 [SMILES: *CC#C] |
| Azide containing DNA, protein, sugar or lipid | 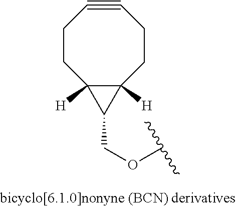 [SMILES: *OC[C@@H]1[C@@H]2CCC#CCC[C@@H]21] |
| Terminal alkyne containing DNA, protein, sugar or lipid | 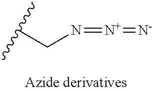 [SMILES: *CN=[N+]=[N-]] |
| Trans- cyclooctene (TCO) containing DNA, protein, sugar or lipid | 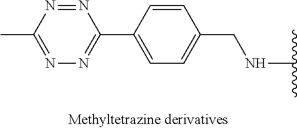 [SMILES: *NCc1ccc(-c2nnc(C)nn2)cc1] |
| Methyltetrazine containing DNA, protein, sugar or lipid | 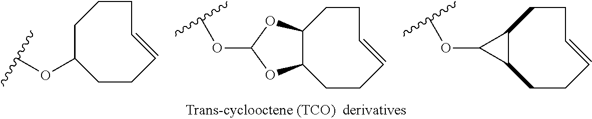 |

In some specific applications, the ligand of the rhodamine 4′-isomer derivative which is capable to interact with or bind to other molecules is benzylguanine and the protein is SNAP-tag, or the ligand is benzylcytosine and the proteine is CLIP-tag, or the ligand is a primary alkyl chloride and the protein is Halo-tag, or the ligand is trimethoprim and the protein is dihydrofolate reductase.

The fluorescent dyes may be used as such or as conjugates with various organic substances, e.g. an amino acid (naturally occurring or not), a peptide, protein, in particular an enzyme or antibody, tubulin or actin, a nucleotide, nucleic acid sequence, including DNA and RNA, a lipid, carbohydrate, an organic or inorganic polyphosphate, a (pharmaceutical) drug or its metabolite, a toxin, NH₄⁺, a metal ion, in particular, Li⁺, Na⁺, K⁺, Cs⁺, Rb⁺, Cu⁺, Tl⁺, Hg⁺, Ag⁺, Au⁺, Ca²⁺, Ba²⁺, Sr²⁺, Be²⁺, Zn²⁺, Ni²⁺, Cu²⁺, Mg²⁺, Co²⁺, Fe²⁺, Mn²⁺, Pt²⁺, Cd²⁺, Hg²⁺, Sn²⁺, Pb²⁺, Au⁺, Cr³⁺, Co³⁺, Fe³⁺, Al³⁺, Ga³⁺, In³⁺, Sc³⁺, Ti³⁺, Tl³⁺, V³⁺, Y³⁺, La³⁺, Pt⁴⁺, Pb⁴⁺, Ce⁴⁺, Ge⁴⁺, Th⁴⁺, Zr⁴⁺, U⁴⁺, or complex thereof.

The conjugation may comprise formation of at least one covalent chemical bond or at least one molecular complex with a chemical entity or substance, such as amine, carboxylic acid, aldehyde, alcohol, aromatic compound, heterocycle, dye, amino acid, amino acid residue coupled to any chemical entity or molecule, in particular as listed above.

More specifically, the conjugation may comprise formation of at least one covalent chemical bond or at least one molecular complex with a chemical compound by a reaction or association with a functional group present in this compound such as amine, carboxylic acid, aldehyde, alcohol, aromatic compound, heterocycle, dye, amino acid, amino acid residue coupled to any chemical compound of interest, in particular as listed above.

In a preferred embodiment of the present invention, the fluorescent dyes as described above or their conjugates are used as cell permeable substances penetrating through membranes of living and fixed cells in vivo or in vitro.

In one specific embodiment of the present invention, the fluorescent dyes as described above or their conjugates are used as such or after photoactivation for tracking and monitoring dynamic processes in a sample or in an object.

In a more specific embodiment, a rhodamine 4′-isomer derivative coupled to tubulin, DNA, RNA, lipid, or actin is used for cell cycle monitoring in living cells or living tissues.

In another specific embodiment, rhodamine 4′-isomers derivatives having a high singlet oxygen (¹O₂) quantum yield, preferably at least 5%, are used for chromophore-assisted light inactivation (CALI) and photodynamic therapy (PDT) in living cells and in living organisms such as humans, mammals, birds, fish, hemichordates, molluscs, tunicates, cnidarians, cephalochordates, flatworms, nematodes, annelids, tardigrades, reptiles, arthropods, echinoderms, chaetognathas, rotifers, frogs, plants, sponges or fungi.

The fluorescent dyes or their conjugates may be used as labels in microscopic, spectroscopic and other imaging techniques, in particular fluorescence microscopy and spectroscopy, in microfluidic devices, capillary electrophoresis, fluorescence activated cell sorting, DNA sequencing, sequence-specific genome labeling, analyte tracking techniques in vitro or in vivo.

Said imaging techniques comprise, e.g., stimulated emission depletion microscopy [STED], single molecule spectroscopy, single molecule switching (SMS) “nanoscopy” (diffraction unlimited optical resolution by using switching of the fluorescence of the single molecules, such as single molecule localization microscopy [SMLM], structured illumination microscopy (SIM), light-sheet microscopy, photoactivation localization microscopy [PALM, PALMIRA, fPALM], stochastic optical reconstruction microscopy [STORM]), fluorescence correlation spectroscopy [FCS] or fluorescence anisotropy spectroscopy, fluorescence recovery after photobleaching [FRAP], fluorescence lifetime imaging [FLIM], ground state depletion with individual molecular return [GSDIM], and fluorescence resonant energy transfer [FRET], correlative fluorescence-electron microscopy, correlative fluorescence—cryo-electron microscopy, microscale thermophoresis, fluorescence in situ hybridization (FISH), nuclear magnetic resonance spectroscopy.

Said analyte tracking techniques using a rhodamine 4′-isomer derivative of the present invention may be performed in vitro or in vivo and may be applied for tracking or monitoring a wide variety of analytes, in particular components of biological cells, such as proteins, lipids, carbohydrates, nucleic acids, sterols, nucleosides, nucleotides, polyphosphates, amino acids, polyamino acids, terpenes, lignines, chitin, chitosan, peptidoglycans, teichoic acids, lipoteichoic acid and vitamins.

In some exemplary and typical embodiments, a method or technique for tracking analytes using a rhodamine 4′-isomer derivative of the present invention comprises at least one of the following sequences of steps:
interacting with or binding of the rhodamine 4′-isomer derivative to the analyte, which is a metal ion, resulting in metal ion chelation and in a corresponding change in fluorescence properties of the rhodamine 4′-isomer derivative, and using this change in fluorescence properties for metal ion imaging in vitro or in vivo, in particular in living cells and in living organisms such as humans, mammals, birds, fish, hemichordates, molluscs, tunicates, cnidarians, cephalochordates, flatworms, nematodes, annelids, tardigrades, reptiles, arthropods, echinoderms, chaetognathas, rotifers, frogs, plants, sponges or fungi;
reacting of the rhodamine 4′-isomer derivative with reactive oxygen species (ROS) resulting in a corresponding change in fluorescence properties of the rhodamine 4′-isomer derivative, and using this change in fluorescence properties for ROS imaging in vitro or in vivo, in particular in living cells and in living organisms such as humans, mammals, birds, fish, hemichordates, molluscs, tunicates, cnidarians, cephalochordates, flatworms, nematodes, annelids, tardigrades, reptiles, arthropods, echinoderms, chaetognathas, rotifers, frogs, plants, sponges or fungi;
selectively interacting or reacting of the rhodamine 4′-isomer derivative with an enzyme, resulting in a corresponding change in fluorescence properties of the rhodamine 4′-isomer derivative, and using this change in fluorescence properties for detection, quantification and imaging of enzymatic activity in vitro and in vivo;
selectively interacting or reacting of the rhodamine 4′-isomer derivative with a lipid, organic and inorganic polyphosphate, protein, carbohydrate, metabolites, DNA or RNA, resulting in a corresponding change in fluorescence properties, and using this change in fluorescence properties for lipid, polyphosphate, protein, carbohydrate, metabolite, DNA or RNA imaging in vitro or in vivo;
interacting or reacting of rhodamine 4′-isomers derivatives coupled to any drug or drug candidate with a target molecule or target site in a cell or tissue, resulting in a corresponding change in fluorescence properties, and using this change in fluorescence properties for drug-target interaction monitoring using fluorescence imaging or NMR in vitro or in vivo;
interacting or reacting of a rhodamine 4′-isomer derivative with oxygen, fluoride or glucose through non-covalent complex or covalent bond formation, resulting in a corresponding change in fluorescence properties of the rhodamine 4′-isomer derivative, and using this change in fluorescence properties for oxygen, fluoride or glucose sensing in vitro or in vivo, in particular in living cells and in living organisms, such as humans, mammals, birds, fish, hemichordates, molluscs, tunicates, cnidarians, cephalochordates, flatworms, nematodes, annelids, tardigrades, reptiles, arthropods, echinoderms, chaetognathas, rotifers, frogs, plants, sponges or fungi.

### BRIEF DESCRIPTION OF THE FIGURES

FIG. 1 illustrates the neighboring group effect in the 4′ isomers of rhodamines: a, Neighboring group effect shifts spirolactone-zwitterion equilibrium of rhodamines; b, Chemical shift differences of amide proton of TMR-LTX regioisomeric probes; c, Comparison of retention times of TMR-LTX regioisomeric probes in HPLC analysis with a SB-C18 column and isocratic elution conditions.

FIG. 2 shows a) graphs representing the absorbance of TMR-LTX positional isomers at λₘₐₓ versus dielectric constant (D) of 1,4-dioxane-water mixtures; b) ^{Dye}D₅₀ values of positional isomers of TMR-COOH and ^{probe}D₅₀ of TMR-LTX.

FIG. 3 illustrates the tubulin labelling of mammalian cells: a, structures of the tubulin probes. b, Wide-field fluorescence microscopy of living primary fibroblasts stained with 100 nM TMR-LTX isomers for 1 h at 37° C.; Phase contrast image is shown at the bottom. c, Quantification of fluorescence signal in the cytoplasm of living cells stained with tubulin probes; d, Cytotoxicity of tubulin fluorescent probes presented as half maximal effective concentration (EC50) after 24 h incubation at 37° C. in growth media.

FIG. 4 shows the performance of DNA and actin fluorescent probes based on rhodamines' positional isomers: a, Structure of DNA probes showing attachment point positional isomerism; b, Wide-field microscopy images of living primary fibroblasts stained with 100 nM 4/5/6-580CP-Hoechst for 1 h at 37° C. Phase contrast image is shown at the bottom; c, Quantification of DNA probes' fluorescence signal in the nuclei; d, Structure of actin probes showing attachment point positional isomerism; e, Wide-field microscopy images of the phase contrast (bottom) and fluorescence channel (top). Living primary fibroblasts stained with 100 nM 4/5/6-610CP-JAS for 1 h at 37° C.; f, Quantification of 4/5/6-610CP-JAS fluorescence signal in the cytoplasm of living cells.

FIG. 5 shows the reactivity of the Halo-tag substrates based on 610CP positional isomers in living U2OS cells and in the cell extracts. The cells express genome-encoded vimentin-Halo-tag protein. The samples were fractionated on 4-15% gradient SDS-PAGE and vimentin-Halo-tag was visualized by fluorescence scanning in red channel. To compare the input, the image of the same gel stained with Coomassie blue is shown.

FIG. 6 shows STED nanoscopy imaging of living cells stained with rhodamine 4′-isomer probes: a, Confocal and STED microscopy images of microtubules in living human fibroblasts stained with 100 nM 4-610CP-CTX for 1 h at 37° C.; b, Confocal and STED microscopy images of microtubules in living human fibroblasts stained with 100 nM 4-SiR-CTX for 1 h at 37° C.; c, Deconvolved STED image of human female primary fibroblast nucleus stained with 100 nM 4-580CP-Hoechst showing the inactivated X chromosome (Xi). Inserts—confocal and STED microscopy zoomed-in images of Xi region; d, Two-colour STED no-wash image of primary human fibroblasts stained with 100 nM 4-610CP-JAS and 10 nM 4-TMR-LTX for 1 h at 37° C.

The following Examples are provided to illustrate the present invention in more detail, however, without limiting the invention to the specific conditions and parameters thereof.

### General Materials and Methods

All chemical reagents and solvents for synthesis were purchased from commercial suppliers (Sigma-Aldrich, Fluka, Acros) and were used without further purification. The composition of mixed solvents is given by the volume ratio (v/v).

NMR spectra were recorded at 25° C. with an Agilent 400-MR spectrometer at 400.06 MHz (¹H) and 100.60 MHz (13C), Bruker Avance III HD 500 spectrometer (av500) at 500.25 MHz (¹H) and 125.80 MHz (¹³C), Varian Mercury Plus 300 spectrometer at 300.14 MHz (¹H), Varian INOVA 600 (1600) spectrometer at 599.74 MHz (¹H) and are reported in ppm. All ¹H and ¹¹C spectra are referenced to tetramethylsilane (5=0 ppm) using the residual signals of the solvents according to the values reported in literature (Gottlieb H. E.; Kotlyar V.; Nudelman A., J Org Chem 1997, 62, 7512-7515). Multiplicities of signals are described as follows: s=singlet, d=doublet, t=triplet, q=quartet, p=pentet, m=multiplet or overlap of non-equivalent resonances; br=broad signal. Coupling constants (J) are given in Hz.

ESI-MS were recorded on a Varian 500-MS spectrometer (Agilent). ESI-HRMS were recorded on a MICROTOF spectrometer (Bruker) equipped with ESI ion source (Apollo) and direct injector with LC autosampler Agilent RR 1200. Liquid chromatography: Analytical LC-MS analysis was performed on an Agilent 1260 Infinity II LC/MS system equipped with an autosampler, diode array detector WR, fluorescence detector Spectra and Infinity Lab LC/MSD 6100 series quadruple with API electrospray. Analysis was done by using an Agilent Zorbax SB-C18 RRHT, 2.1×50 mm, 1.8 μm threaded column and SUPELCO Titan C18, 2.1×75 mm, 1.9 μm column with A: 25 mM HCOONH₄ (pH=3.6) aqueous buffer and B: MeOH

Preparative HPLC was performed on an Interchim puriFlash 4250 2× preparative HPLC/Flash hybrid system (Article No. 1I5140, Interchim) with a 2 mL/5 mL injection loop, a 200-600 nm UV-Vis detector and an integrated ELSD detector (Article No. 1A3640, Interchim). Preparative column: Eurospher II 100-5 C18 5 μm, 250×20.0 mm (Article No.: 25PE181E2J, Knauer), typical flow rate: 25 mL/min, unless specified otherwise. Analytical TLC was performed on Merck Millipore ready-to-use plates with silica gel 60 (F254) (Cat. No. 1.05554.0001). Flash chromatography was performed on Biotage Isolera flash purification system using the type of cartridge and solvent gradient indicated.

### Maintenance and Preparation of Cells

Human primary dermal fibroblasts and HeLa cells were cultured in high-glucose DMEM (Life Technologies, Cat. No. 31053-028) supplemented with GlutaMAX-1 (Life Technologies, Cat. No. 35050-038) and 10% fetal bovine serum (FBS, Life Technologies, Cat. No. 10270-106) in a humidified 5% CO₂ incubator at 37° C. The cells were split every 3-4 days or at confluence. Cells were seeded in glass bottom 12-well plates (MatTek Corporation, Cat. No. P12G-1.0-14-F).

Cells were stained with the fluorescent probes in DMEM (Thermo Fisher Scientific, Cat. No. 31053-028) supplemented with 10% FBS (Thermo Fisher Scientific, Cat. No. 10082139) at 37° C. and 5% CO₂. If needed, the cells were washed 2 times with HBSS (Hanks' balanced salt solution, Lonza, Cat. No. BE10-527F) and imaged in DMEM with 10% FBS. No-wash experiments were performed in DMEM with 10% FBS after probe addition and incubation for the indicated period of time. For Airyscan experiments, cells were seeded in a 10-well plate (Greiner bio-one culture slides, PS, 75/25 mm. (Art. Nr.:543079)) 1 day before staining. The probes were applied to the cells in DMEM medium and after 1 h or overnight incubation cells were imaged without washing.

STED Microscope with 775 nm Depletion Laser

Comparative confocal and STED images were acquired on Abberior STED 775 QUAD scanning microscope (Abberior Instruments GmbH, Germany) equipped with 561 nm and 640 nm 40 MHz pulsed excitation lasers, a pulsed 775 nm 40 MHz STED laser, and an UPlanSApo 100×/1.40 Oil objective. The following detection windows were used: TMR/580CP channel 615/20 nm and 610CP/SiR channel 685/70 nm. In this setup, voxel size was 15-30 nm in xy plane and 150 nm in z-axis for STED images.

### Measurements of Absorbance Spectra in 1,4-Dioxane-Water Mixtures

Measurements of the absorbance changes in 1,4-dioxane-water mixtures were performed by pipetting 2 μL stock solutions of dyes or probes in DMSO into a 96 glass bottom well plate (11 wells per sample) made from propylene (Corning 3364). To the wells going from right to left 300 μL of 1,4-dioxane-water mixtures containing 100%, 90%, 80%, 70%, 60%, 50%, 40%, 30%, 20%, 10% or 0% 1,4-dioxane was added (if needed, mixtures with 0.3% SDS are used, with an exception in 100% dioxane due to solubility issues). After incubation for 1 hour at room temperature, absorption of solutions in each well was recorded from 320 nm to 850 nm with wavelength step size of 1 nm on a multiwell plate reader Spark® 20M (Tecan). The background absorption of the glass bottom plate was measured in wells containing only 1,4-dioxane-water mixture with similar amount of DMSO and subtracted from the spectra of the samples. Plots of λₘₐₓ versus dielectric constant (D) of 1,4-dioxane-water mixtures (G. Åkerlöf, A. O. Short, The Dielectric Constant of Dioxane-Water Mixtures between 0 and 80°, J. Am. Chem. Soc. 1936 58(7) 1241-1243). D₅₀ value was obtained by fitting to dose-response equation EC₅₀ as implemented in GraphPad 6.0 software:

$$A = {A}_{0} + ( {A}_{m a x} - {A}_{0} ) / ( 1 + {( \frac{{D}_{50}}{d} )}^{Hill} ) \qquad ( 1 )$$

where A₀—absorbance at λₘₐₓ at εᵣ=0, λₘₐₓ—the highest reached absorbance at λₘₐₓ. d—dielectric constant of 1,4-dioxane-water mixture at a given point, Hill—Hill slope coefficient determining the steepness of a dose-response curve, D₅₀—corresponds to d value that provokes half of the absorbance amplitude (λₘₐₓ−A₀).

### Cell Cycle Analysis by Imaging Flow Cytometry and EC₅₀ Determination

HeLa cells were grown in 6-well plates (^{˜}250.000 cells per well) for 24 h in the presence of the fluorescent probe in variable concentrations. The probes were dissolved in DMSO at 500-2000-fold stock concentration and added to the media of cultured cells at 500-2000-fold dilution accordingly. In parallel, the appropriate DMSO control samples were prepared by adding corresponding amount of DMSO volume to the separate well. Cells were processed according to the NucleoCounter® NC-3000™ two-step cell cycle analysis protocol for cells attached to T-flasks, cell culture plates or micro-carriers. In particular, the 250 μl lysis solution (Solution 10, Chemometec Cat. No. 910-3010) supplemented with 10 μg/ml DAPI (Solution 12, Chemometec Cat. No. 910-3012) was used per well, incubated at 37° C. for 5 min. Then 250 μl of stabilization solution (Solution 11, Chemometec Cat. No. 910-3011) was added. Cells were counted on a NucleoCounter® NC-3000™ in NC-Slide A2™ slides (Chemometec, Cat. No. 942-0001) loaded with ˜30 μl of each of the cell suspensions into the chambers of the slide. Each time, ^{˜}10.000 cells in total were measured, and the obtained cell cycle histograms were analysed with ChemoMetec NucleoView NC-3000 software, version 2.1.25.8. All experiments were repeated three times and the results are presented as means with standard deviations. The EC₅₀ values were determined by plotting the percentage of cells in subG1 phase and fitting the curve in GraphPad Prism 6 to the following function:

$$Y = {Y}_{m i n} + ( {Y}_{m a x} - {Y}_{m i n} ) / ( 1 + {( \frac{{EC}_{50}}{X} )}^{Hill} ) \qquad ( 2 )$$

where Yₘᵢₙ—cells population percentage in subG1 phase then no probe was added, Yₘₐₓ—highest reachable percentage of cells in subG1 phase and shared value for all data sets equal to 69%. X—cells population percentage in subG1 phase then added probe is at X concentration, Hill—Hill slope coefficient determining the steepness of a dose-response curve, EC₅₀—the concentration of probe that provoking halfway of subG1 cells in a population between the baseline (Yₘᵢₙ) and maximum response (Yₘₐₓ).

### Processing and Visualization of Acquired Images

All acquired or reconstructed images were processed and visualized using Fiji (J. Schindelin, I. Arganda-Carreras, E. Frise, V. Kaynig, M. Longair, T. Pietzsch, S. Preibisch, C. Rueden, S. Saalfeld, B. Schmid, J. Y. Tinevez, D. J. White, V. Hartenstein, K. Eliceiri, P. Tomancak, A. Cardona, Fiji: an open-source platform for biological-image analysis, Nat Methods 2012 9(7) 676-82). Line profiles were measured using the “straight line” tool with the line width set to 3 pixels.

For the signal measurements, image files were converted to TIF file using Fiji and analyzed with CellProfiler 3.1.8 (A. E. Carpenter, T. R. Jones, M. R. Lamprecht, C. Clarke, I. H. Kang, O. Friman, D. A. Guertin, J. H. Chang, R. A. Lindquist, J. Moffat, P. Golland, D. M. Sabatini, CellProfiler: image analysis software for identifying and quantifying cell phenotypes, Genome Biol 2006 7(10) R100), where the pipeline identified the nuclear region and measured the mean signal in this region. Background signal was measured in the region which is 3 pixels (450 nm) away from the nuclear border and 7 pixels (1050 nm) wide. The background subtracted signal was processed with GraphPad Prism 6.

Actin/tubulin cytosolic signal was estimated using CellProfiler v.3.1.8. Briefly, probe channel was smoothed using median filter, nuclei were identified in DAPI channel and were used as seeds to find cell outlines in a smoothed actin channel. Background was defined as a lower quartile of pixel intensity in the area not covered by cells in the original probe channel. The background was subtracted from the original probe channel, and actin/tubulin staining was measured as mean pixel intensity per object in background-corrected probe channel. Statistical analysis performed by GraphPad Prism 6.

### Labeling of Halo-Tagged Vimentin in Living Cells and Extracts

For assessing living cell staining, U2OS cells expressing vimentin-Halo-tag were grown on 35 mm diameter plastic plates in DMEM with 10% FBS. 100 nM x-610CP-Halo substrates were added and the cells were incubated at 37° C. for 1 h. Then, the cells were washed twice with growth medium and twice with HBSS. 200 μl of CelLytic M solution (Sigma), supplemented with complete EDTA-free protease inhibitor cocktail (Sigma) was added, and the cells were allowed to lyse for 15 min. at room temperature with gentle agitation. The solution was collected and centrifuged for 15 min. at 13000 rpm (4° C.). 180 μl of supernatant was mixed with 60 μl 4×SDS-PAGE sample buffer and boiled for 5 min.

To assess reactivity in the cell extracts, the cells were lysed as described above, and the lysates were incubated with 100 nM HaloTag substrates for 30 min. at 37° C. before boiling. The samples were fractionated in 4-15% gradient.

### Synthesis Example A

### Synthesis of Rhodamine 4′-Isomer Dyes

### A.1 Di-tert-butyl 3-bromophthalate (1)

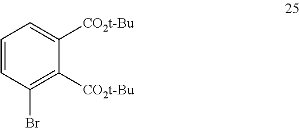 [SMILES: CC(C)(C)OC(=O)c1cccc(Br)c1C(=O)OC(C)(C)C]

3-Bromophtalic acid (1.0 g, 4.08 mmol) was suspended in DCM (15 mL) in a sealable pressure glass tube. The mixture was cooled in a NaCl/ice bath and ^{˜}10 mL of isobutylene gas was condensed in the mixture. Catalytic amount of concentrated sulphuric acid (0.1 mL, 1.87 mmol) was added to the stirred and cooled reaction mixture and the pressure tube was tightly sealed. Reaction mixture was stirred at room temperature for 48 h, during this time the suspension became a clear solution. Then reaction mixture was cooled in ice bath and the tube was carefully opened with release of pressure. The resulting solution was poured to saturated NaHCO₃ solution (50 mL), extracted with DCM (2×30 mL). The organic extracts were combined and washed with water and brine, dried over Na₂SO₄. The product was isolated by flash column chromatography (Teledyne Isco RediSep Rf 40 g, isocratic hold 5% of EtOAc in Hexane), fractions containing the product were evaporated to give 1.02 g (70%) of white solid.

¹H NMR (400 MHz, cdcl₃) δ 7.79 (dd, J=7.8, 1.1 Hz, 1H), 7.67 (dd, J=8.0, 1.1 Hz, 1H), 7.23 (t, J=7.9 Hz, 1H), 1.61 (s, 9H), 1.56 (s, 9H).

¹³C NMR (101 MHz, cdcl₃) δ 165.9, 163.6, 137.9, 136.1, 131.6, 129.5, 128.5, 120.3, 83.0, 82.1, 28.1, 28.0.

ESI-MS, positive mode: m/z=357.1, 359.1 [M+H]⁺.

HRMS (ESI) calcd for C₁₆H₂₂N₂BrO₄ [M+H]⁺ 357.0696, found 357.0698.

### A.2 Tert-butyl 3′,6′-bis((tert-butyldimethylsilyl)oxy)-3-oxo-3H-spiro[isobenzofuran-1,9′-xanthene]-4-carboxylate (5)

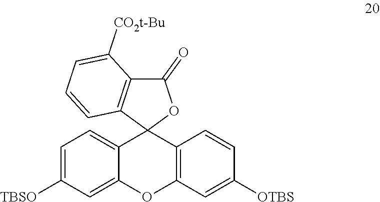 [SMILES: CC(C)(C)OC(=O)c1cccc2c1C(=O)OC21c2ccc(O[Si](C)(C)C(C)(C)C)cc2Oc2cc(O[Si](C)(C)C(C)(C)C)ccc21]

In a 25 mL round-bottom flask, a degassed solution of 1 (740 mg, 2.07 mmol, 2 eq.) in anhydrous THF (6 mL) and pentane (4 mL) was cooled to ^{˜}−116° C. (diethyl ether-liquid N₂). n-Butyllithium (1.3 mL of 1.6 M solution in hexanes, 2.07 mmol, 2 eq.) was carefully introduced through a needle. Clear solution quickly turned orange and then deep brown; it was stirred at ^{˜}−116° C. for 10 min, and the solution of ketone 2 (475 mg, 1.04 mmol, 1 eq.) (Martinez-Peragon, A.; Miguel, D.; Jurado, R.; Justicia, J.; Alvarez-Pez, J. M.; Cuerva, J. M.; Crovetto, L., Chemistry, 2014, 20 (2), 447-55) in THF (3 mL) was injected along the wall of the flask. Stirred at ^{˜}−116° C. for 10 minutes and then flask was taken out of the cooling bath and left to warm to rt and stirred for further 30 min. The reaction mixture was quenched with water (10 mL), adjusted to pH ^{˜}5 with acetic acid, extracted with ethyl acetate (3×30 mL), the combined organic layers were washed with brine and dried over Na₂SO₄. The products were isolated by flash column chromatography (Büchi Reveleris HP silica 40 g; gradient 0% to 20% ethyl acetate-hexane.) In some cases additional purification was needed (Teledyne Isco RediSep Rf 40 g, gradient 20% to 100% of DCM-Hexane). Product yield 433 mg (63%) of off-white solid.

¹H NMR (400 MHz, d₆-acetone) δ 7.86 (t, J=7.6 Hz, 1H), 7.82 (dd, J=7.6, 1.4 Hz, 1H), 7.40 (dd, J=7.6, 1.4 Hz, 1H), 6.81 (d, J=2.4 Hz, 2H), 6.76 (d, J=8.6 Hz, 2H), 6.70 (dd, J=8.6, 2.4 Hz, 2H), 1.66 (s, 9H), 1.00 (s, 18H), 0.27 (s, 12H).

¹³C NMR (101 MHz, d₆-acetone) δ 165.9, 165.0, 157.6, 153.9, 152.1, 135.2, 133.1, 129.3, 129.1, 126.0, 123.2, 116.9, 112.4, 107.4, 82.4, 81.3, 27.2, 25.0, 17.9, −5.3.

ESI-MS, positive mode: m/z=661.3 [M+H]⁺.

HRMS (ESI) calcd for C₃₇H₄₉O₇Si₂ [M+H]⁺ 661.3011, found 661.3004.

### A.3 Tert-butyl 3,6-bis((tert-butyldimethylsilyl)oxy)-10,10-dimethyl-3′-oxo-3′H,10H-spiro[anthracene-9,1′-isobenzofuran]-4′-carboxylate (6)

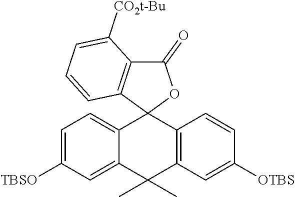 [SMILES: CC(C)(C)OC(=O)c1cccc2c1C(=O)OC21c2ccc(O[Si](C)(C)C(C)(C)C)cc2C(C)(C)c2cc(O[Si](C)(C)C(C)(C)C)ccc21]

In a 25 mL round-bottom flask, a degassed solution of 1 (740 mg, 2.07 mmol, 2 eq.) in anhydrous THF (6 mL) and pentane (4 mL) was cooled to ^{˜}−116° C. (diethyl ether-liquid N₂). n-Butyllithium (1.3 mL of 1.6 M solution in hexanes, 2.07 mmol, 2 eq.) was carefully introduced through a needle. Clear solution quickly turned orange and then deep brown; it was stirred at ^{˜}−116° C. for 10 min, and the solution of ketone 3 (502 mg, 1.04 mmol, 1 eq.) (Grimm, J. B.; Sung, A. J.; Legant, W. R.; Hulamm, P.; Matlosz, S. M.; Betzig, E.; Lavis, L. D., ACS Chem Biol, 2013, 8(6), 1303-1310) in THF (3 mL) was injected along the wall of the flask. Stirred at ^{˜}−116° C. for 10 minutes and then flask was taken out of the cooling bath and left to warm to rt and stirred for further 30 min. The reaction mixture was quenched with water (10 mL), adjusted to pH ^{˜}5 with acetic acid, extracted with ethyl acetate (3×30 mL), the combined organic layers were washed with brine and dried over Na₂SO₄. The products were isolated by flash column chromatography (Büchi Reveleris HP silica 40 g; gradient 0% to 20% ethyl acetate-hexane.) In some cases additional purification was needed (Teledyne Isco RediSep Rf 40 g, gradient 20% to 100% of DCM-Hexane). Product yield 471 mg (66%) of off-white solid.

¹H NMR (600 MHz, cdcl₃) δ 7.72 (dd, J=7.6, 0.8 Hz, 1H), 7.58 (t, J=7.6 Hz, 1H), 7.07 (d, J=0.8 Hz, 1H), 7.05 (d, J=2.0 Hz, 2H), 6.66 (d, J=8.6 Hz, 2H), 6.61 (dd, J=8.6, 2.0 Hz, 2H), 1.79 (s, 3H), 1.71 (s, 3H), 1.70 (s, 9H), 0.98 (s, 18H), 0.21 (s, 12H).

¹³C NMR (126 MHz, cdcl₃) δ 167.3, 165.3, 156.4, 156.1, 146.6, 134.1, 133.0, 129.2, 128.9, 125.7, 124.1, 123.2, 118.9, 117.4, 85.0, 83.3, 38.0, 34.9, 33.1, 28.1, 25.7, 18.3, −4.2,

ESI-MS, positive mode: m/z=687.4 [M+H]⁺.

HRMS (ESI) calcd for C₄₀H₅₅O₆Si₂ [M+H]⁺ 687.3532, found 687.3523.

### A.4 Tert-butyl 3,7-bis((tert-butyldimethylsilyl)oxy)-5,5-dimethyl-3′-oxo-3′H,5H-spiro[dibenzo[b,e]siline-10,1′-isobenzofuran]-4′-carboxylate (7)

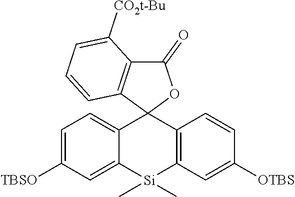 [SMILES: CC(C)(C)OC(=O)c1cccc2c1C(=O)OC21c2ccc(O[Si](C)(C)C(C)(C)C)cc2[Si](C)(C)c2cc(O[Si](C)(C)C(C)(C)C)ccc21]

2.07 mmol, 2 eq.) in anhydrous THF (6 mL) and pentane (4 mL) was cooled to ^{˜}−116° C. (diethyl ether-liquid N₂). n-Butyllithium (1.3 mL of 1.6 M solution in hexanes, 2.07 mmol, 2 eq.) was carefully introduced through a needle. Clear solution quickly turned orange and then deep brown; it was stirred at ^{˜}−116° C. for 10 min, and the solution of ketone 4 (520 mg, 1.04 mmol, 1 eq.) (Butkevich, A. N.; Belov, V. N.; Kolmakov, K.; Sokolov, V. V.; Shojaei, H.; Sidenstein, S. C.; Kamin, D.; Matthias, J.; Vlijm, R.; Engelhardt, J.; Hell, S. W., Chemistry—A European Journal, 2017, 23(50), 12114-12119) in THF (3 mL) was injected along the wall of the flask. Stirred at ^{˜}−116° C. for 10 minutes and then flask was taken out of the cooling bath and left to warm to rt and stirred for further 30 min. The reaction mixture was quenched with water (10 mL), adjusted to pH ^{˜}5 with acetic acid, extracted with ethyl acetate (3×30 mL), the combined organic layers were washed with brine and dried over Na₂SO₄. The products were isolated by flash column chromatography (Büchi Reveleris HP silica 40 g; gradient 0% to 20% ethyl acetate-hexane.) In some cases additional purification was needed (Teledyne Isco RediSep Rf 40 g, gradient 20% to 100% of DCM-Hexane). Product yield 446 mg (61%) of off-white solid.

¹H NMR (400 MHz, cdcl₃) δ 7.69 (dd, J=7.6, 1.0 Hz, 1H), 7.62 (t, J=7.6 Hz, 1H), 7.31 (dd, J=7.6, 1.0 Hz, 1H), 7.09 (d, J=2.7 Hz, 2H), 6.87 (dd, J=8.7, 0.4 Hz, 2H), 6.67 (dd, J=8.7, 2.7 Hz, 2H), 1.65 (s, 9H), 0.96 (s, 18H), 0.60 (s, 3H), 0.56 (s, 3H), 0.17 (s, 12H).

¹³C NMR (101 MHz, cdcl₃) δ 167.5, 165.6, 155.2, 155.2, 137.2, 136.9, 134.0, 133.5, 128.9, 128.6, 126.3, 125.0, 123.1, 121.2, 89.1, 83.4, 28.1, 25.7, 18.3, −1.3, −2.9, −4.3, −4.

ESI-MS, positive mode: m/z=703.3 [M+H]⁺.

HRMS (ESI) calcd for C₃₉H₅₅O₆Si₃ [M+H]⁺ 703.3301, found 703.3304.

### A.5 Tert-butyl 3′,6′-dihydroxy-3-oxo-3H-spiro[isobenzofuran-1,9′-xanthene]-4-carboxylate (8)

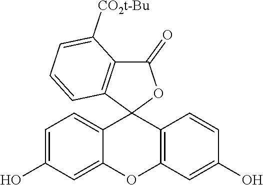 [SMILES: CC(C)(C)OC(=O)c1cccc2c1C(=O)OC21c2ccc(O)cc2Oc2cc(O)ccc21]

To a cooled (ice-water bath) solution of corresponding compound 5 (420 mg, 0.635 mmol, 1 eq.) in THF (15 mL) tetrabutylammonium fluoride trihydrate (800 mg, 2.54 mmol, 4 eq.) solution in THF (5 mL) was added. The resulting intensively coloured solution was stirred at 0° C. for 1 h. Sat. aq. NH₄Cl (20 mL) was added followed by minimal amount of water necessary to dissolve the solids, the mixture was extracted with ethyl acetate (3×30 mL), the combined organic layers were washed with brine and dried over Na₂SO₄. The products were isolated by flash column chromatography (Teledyne Isco RediSep Rf 24 g; gradient 2% to 30% ethyl acetate-CH₂Cl₂) and evaporated to obtain viscous oil which solidifies overtime. Product yield 214 mg (78%) of orange solid material.

¹H NMR (400 MHz, d₆-dmso) δ 10.15 (s, 2H), 7.87-7.76 (m, 2H), 7.39 (dd, J=7.3, 1.4 Hz, 1H), 6.69 (d, J=2.0 Hz, 2H), 6.63-6.47 (m, 4H), 1.60 (s, 9H).

¹³C NMR (101 MHz, d₆-dmso) δ 166.1, 164.9, 159.5, 153.4, 151.9, 135.6, 132.2, 129.2, 128.9, 126.3, 122.6, 112.7, 109.3, 102.3, 82.6, 82.2, 27.7.

ESI-MS, positive mode: m/z=433.1 [M+H]⁺.

HRMS (ESI) calcd for C₂₅H₂₁O₇[M+H]⁺ 433.1282, found 433.1279.

### A.6 Tert-butyl 3,6-dihydroxy-10,10-dimethyl-3′-oxo-3′H,10H-spiro[anthracene-9,1′-isobenzofuran]-4′-carboxylate (9)

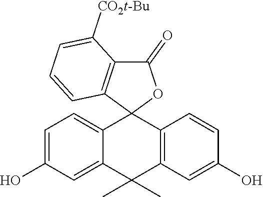 [SMILES: CC(C)(C)OC(=O)c1cccc2c1C(=O)OC21c2ccc(O)cc2C(C)(C)c2cc(O)ccc21]

To a cooled (ice-water bath) solution of corresponding compound 6 (445 mg, 0.647 mmol, 1 eq.) in THF (15 mL) tetrabutylammonium fluoride trihydrate (817 mg, 2.59 mmol, 4 eq.) solution in THF (5 mL) was added. The resulting intensively coloured solution was stirred at 0° C. for 1 h. Sat. aq. NH₄Cl (20 mL) was added followed by minimal amount of water necessary to dissolve the solids, the mixture was extracted with ethyl acetate (3×30 mL), the combined organic layers were washed with brine and dried over Na₂SO₄. The products were isolated by flash column chromatography (Teledyne Isco RediSep Rf 24 g; gradient 2% to 30% ethyl acetate-CH₂Cl₂) and evaporated to obtain viscous oil which solidifies overtime. Product yield 264 mg (89%) of orange solid material.

¹H NMR (300 MHz, cdcl₃) δ 7.73 (dd, J=7.6, 1.0 Hz, 1H), 7.60 (t, J=7.6 Hz, 1H), 7.14 (s, 2H), 7.05 (dd, J=7.6, 1.0 Hz, 1H), 6.98 (d, J=2.4 Hz, 2H), 6.51 (dd, J=8.6, 2.4 Hz, 2H), 6.44 (d, J=8.6 Hz, 2H), 1.68 (s, 9H).

¹³C NMR (126 MHz, cdcl₃) δ 169.0, 165.9, 156.7, 156.2, 147.3, 134.7, 132.7, 129.2, 129.1, 126.1, 123.2, 122.4, 114.8, 112.9, 87.0, 84.1, 38.1, 34.8, 32.3, 28.1.

ESI-MS, positive mode: m/z=459.2 [M+H]⁺.

HRMS (ESI) calcd for C₂₈H₂₇O₆[M+H]⁺ 459.1802, found 459.1805.

### A.7 Tert-butyl 3,7-dihydroxy-5,5-dimethyl-3′-oxo-3′H,5H-spiro[dibenzo[b,e]siline-10,1′-isobenzofuran]-4′-carboxylate (10)

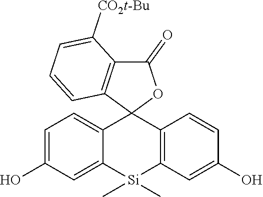 [SMILES: CC(C)(C)OC(=O)c1cccc2c1C(=O)OC21c2ccc(O)cc2[Si](C)(C)c2cc(O)ccc21]

To a cooled (ice-water bath) solution of corresponding compound 7 (420 mg, 0.575 mmol, 1 eq.) in THF (15 mL) tetrabutylammonium fluoride trihydrate (725 mg, 2.3 mmol, 4 eq.) solution in THF (5 mL) was added. The resulting intensively coloured solution was stirred at 0° C. for 1 h. Sat. aq. NH₄Cl (20 mL) was added followed by minimal amount of water necessary to dissolve the solids, the mixture was extracted with ethyl acetate (3×30 mL), the combined organic layers were washed with brine and dried over Na₂SO₄. The products were isolated by flash column chromatography (Teledyne Isco RediSep Rf 24 g; gradient 2% to 30% ethyl acetate-CH₂Cl₂) and evaporated to obtain viscous oil which solidifies overtime. Product yield 254 mg (93%) of orange solid material.

¹H NMR (400 MHz, d₆-dmso) δ 9.74 (s, 2H), 7.83 (t, J=7.6 Hz, 1H), 7.72 (dd, J=7.6, 0.9 Hz, 1H), 7.46 (dd, J=7.6, 0.9 Hz, 1H), 7.16-7.08 (m, 2H), 6.74-6.60 (m, 4H), 1.55 (s, 9H), 0.56 (s, 3H), 0.48 (s, 3H).

¹³C NMR (101 MHz, d₆-dmso) δ 167.4, 165.4, 157.3, 154.6, 137.5, 135.2, 134.5, 133.5, 129.0, 128.6, 127.1, 122.3, 120.7, 117.3, 90.0, 83.0, 28.0, 0.4, −1.4.

ESI-MS, positive mode: m/z=475.2 [M+H]⁺.

HRMS (ESI) calcd for C₂₇H₂₇O₆Si [M+H]⁺ 475.1571, found 475.1573.

### A.8 Tert-butyl 3-oxo-3′,6′-bis(((trifluoromethyl)sulfonyl)oxy)-3H-spiro[isobenzofuran-1,9′-xanthene]-4-carboxylate (11)

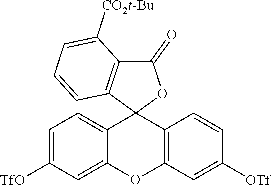 [SMILES: CC(C)(C)OC(=O)c1cccc2c1C(=O)OC21c2ccc(OS(=O)(=O)C(F)(F)F)cc2Oc2cc(OS(=O)(=O)C(F)(F)F)ccc21]

Trifluoromethanesulfonic anhydride 1M solution in DCM (1.85 mL, 1.85 mmol, 4 eq.) was slowly added dropwise to a solution of corresponding compound 8 (200 mg, 0.463 mmol, 1 eq.) and pyridine (0.3 mL, 3.7 mmol; 8 eq.) in dry DCM (10 mL), cooled in ice-water bath. The flask was then removed from the cooling bath, and the mixture was stirred at rt for 1 h. Afterwards, the mixture was diluted with water (30 mL), extracted with CH₂Cl₂ (3×20 mL), the combined extracts were washed with water, brine and dried over Na₂SO₄. The products were isolated by flash column chromatography (Teledyne Isco RediSep Rf 24 g; gradient 5% to 40% ethyl acetate-hexane).

Product yield 255 mg (79%) of white solid material.

¹H NMR (400 MHz, cdcl₃) δ 7.87 (d, J=7.4 Hz, 1H), 7.73 (t, J=7.4 Hz, 1H), 7.30 (d, J=2.2 Hz, 2H), 7.22 (d, J=7.4 Hz, 1H), 7.08-6.99 (m, 4H), 1.70 (s, 9H).

¹³C NMR (101 MHz, cdcl₃) δ 165.4, 164.6, 153.3, 151.2, 150.2, 135.4, 133.7, 130.7, 130.0, 125.7, 122.4, 119.1, 118.6 (q, ¹J_{C-F}=319 Hz, —CF₃), 117.7, 110.7, 83.9, 78.8, 28.0.

ESI-MS, positive mode: m/z=697.0 [M+H]⁺.

HRMS (ESI) calcd for C₂₇H₁₉F₆O₁₁S₂[M+H]⁺ 697.0267, found 697.0249.

### A.9 Tert-butyl 10,10-dimethyl-3′-oxo-3,6-bis(((trifluoromethyl)sulfonyl)oxy)-3′H,10H-spiro[anthracene-9,1′-isobenzofuran]-4′-carboxylate (12)

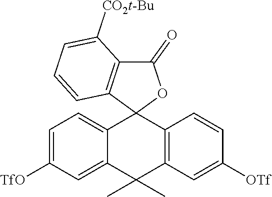 [SMILES: CC(C)(C)OC(=O)c1cccc2c1C(=O)OC21c2ccc(OS(=O)(=O)C(F)(F)F)cc2C(C)(C)c2cc(OS(=O)(=O)C(F)(F)F)ccc21]

Trifluoromethanesulfonic anhydride 1M solution in DCM (2.14 mL, 2.14 mmol, 4 eq.) was slowly added dropwise to a solution of corresponding compound 9 (245 mg, 0.535 mmol, 1 eq.) and pyridine (0.35 mL, 4.3 mmol; 8 eq.) in dry DCM (10 mL), cooled in ice-water bath. The flask was then removed from the cooling bath, and the mixture was stirred at rt for 1 h. Afterwards, the mixture was diluted with water (30 mL), extracted with CH₂Cl₂ (3×20 mL), the combined extracts were washed with water, brine and dried over Na₂SO₄. The products were isolated by flash column chromatography (Teledyne Isco RediSep Rf 24 g; gradient 5% to 40% ethyl acetate-hexane). Product yield 271 mg (70%) of white solid material.

¹H NMR (400 MHz, cdcl₃) δ 7.83 (dd, J=7.6, 0.9 Hz, 1H), 7.68 (t, J=7.6 Hz, 1H), 7.54 (d, J=2.5 Hz, 2H), 7.11 (dd, J=8.8, 2.5 Hz, 2H), 7.07 (dd, J=7.6, 0.9 Hz, 1H), 6.94 (d, J=8.8 Hz, 2H), 1.89 (s, 3H), 1.79 (s, 3H), 1.70 (s, 9H).

¹³C NMR (101 MHz, cdcl₃) δ 166.4, 164.8, 154.8, 150.1, 146.9, 135.1, 133.8, 131.3, 130.3, 130.2, 125.6, 122.6, 120.3, 119.6, 118.9 (q, 1JC-F=319 Hz,—CF3), 83.8, 82.6, 38.8, 34.7, 33.0, 28.0.

ESI-MS, positive mode: m/z=745.1 [M+Na]⁺.

HRMS (ESI) calcd for C₃₀H₂₅O₁₀S₂F₆[M+H]⁺ 723.0788, found 723.0800.

### A.10 Tert-butyl 5,5-dimethyl-3′-oxo-3,7-bis(((trifluoromethyl)sulfonyl)oxy)-3′H,5H-spiro[dibenzo[b,e]siline-10,1′-isobenzofuran]-4′-carboxylate (13)

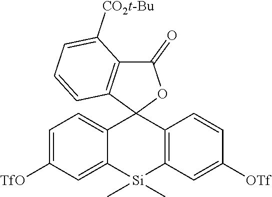 [SMILES: CC(C)(C)OC(=O)c1cccc2c1C(=O)OC21c2ccc(OS(=O)(=O)C(F)(F)F)cc2[Si](C)(C)c2cc(OS(=O)(=O)C(F)(F)F)ccc21]

Trifluoromethanesulfonic anhydride 1M solution in DCM (1.94 mL, 1.94 mmol, 4 eq.) was slowly added dropwise to a solution of corresponding compound 10 (230 mg, 0.484 mmol, 1 eq.) and pyridine (0.31 mL, 3.87 mmol; 8 eq.) in dry DCM (10 mL), cooled in ice-water bath. The flask was then removed from the cooling bath, and the mixture was stirred at rt for 1 h. Afterwards, the mixture was diluted with water (30 mL), extracted with CH₂Cl₂ (3×20 mL), the combined extracts were washed with water, brine and dried over Na₂SO₄. The products were isolated by flash column chromatography (Teledyne Isco RediSep Rf 24 g; gradient 5% to 40% ethyl acetate-hexane). Product yield 271 mg (70%) of white solid material. Product yield 258 mg (72%) of white solid material.

¹H NMR (400 MHz, cdcl₃) δ 7.81 (dd, J=7.6, 1.1 Hz, 1H), 7.75 (t, J=7.6 Hz, 1H), 7.56 (dd, J=2.3, 0.8 Hz, 2H), 7.39 (dd, J=7.6, 1.1 Hz, 1H), 7.24-7.17 (m, 4H), 1.67 (s, 9H), 0.74 (s, 3H), 0.70 (s, 3H).

¹³C NMR (101 MHz, cdcl₃) δ 166.2, 164.8, 152.8, 149.3, 144.0, 138.7, 134.8, 134.3, 130.0, 129.2, 126.3, 126.1, 122.8, 122.6, 118.7 (q, 1JC-F=319 Hz,—CF3), 87.3, 83.8, 28.0, −0.2, −1.7.

ESI-MS, positive mode: m/z=761.0 [M+Na]⁺.

HRMS (ESI) calcd for C₂₉H₂₅O₁₀S₂SiF₆ [M+H]⁺ 739.0557, found 739.0562.

### A.11 Tert-butyl 3′,6′-bis(dimethylamino)-3-oxo-3H-spiro[isobenzofuran-1,9′-xanthene]-4-carboxylate (14)

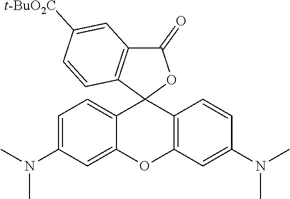

A mixture of Pd₂(dba)₃ (25.6 mg, 0.026 mmol, 0.1 eq.), Xantphos (44.8 mg, 0.077 mmol, 0.3 eq.), Cs₂CO₃ (251 mg, 0.77, 3 eq.) and corresponding triflate 11 (180 mg, 0.258 mmol, 1 eq) in dry 1,4-dioxane (2.5 mL) was degassed on a Schlenk line. Then 2M solution of dimethylamine in THF (323 μL, 0.645 mmol, 2.5 eq.) were introduced. Reaction mixture was stirred in a septa sealed tube at 100° C. under argon for 18 h. Upon cooling, the resulting brown mixture was diluted with water (30 mL), pH adjusted to 5-6 with AcOH and extracted with ethyl acetate (3×30 mL), the combined organic layers were washed with brine and dried over Na₂SO₄. The filtrate was evaporated and the product was isolated by flash chromatography Büchi Reveleris HP silica 24 g; gradient 2% to 30% MeOH-DCM. The fractions containing the product were evaporated, the residue was redissolved in acetonitrile-water (1:1), microfiltered through a 0.45 μm PTFE membrane filter and lyophilized to obtain 67 mg (53%) of pink solid. ¹H NMR (400 MHz, cd₃od) δ 8.00 (dd, J=7.8, 1.3 Hz, 1H), 7.57 (t, J=7.8 Hz, 1H), 7.39 (dd, J=7.8, 1.3 Hz, 1H), 7.31 (d, J=9.5 Hz, 2H), 7.00 (dd, J=9.5, 2.5 Hz, 2H), 6.85 (d, J=2.5 Hz, 2H), 3.26 (s, 12H), 1.61 (s, 9H).

¹³C NMR (101 MHz, cd₃od) δ 173.7, 167.8, 159.1, 158.9, 158.8, 144.0, 133.6, 133.3, 131.9, 131.8, 131.6, 128.1, 115.3, 114.9, 97.3, 83.3, 40.9, 28.3.

ESI-MS, positive mode: m/z=487.2 [M+H]⁺.

HRMS (ESI) calcd for C₂₉H₃₁N₂O₅ [M+H]⁺ 487.2227, found 487.2232

### A.12 Tert-butyl 10,10-dimethyl-3,6-bis(methylamino)-3′-oxo-3′H,10H-spiro[anthracene-9,1′-isobenzofuran]-4′-carboxylate (15)

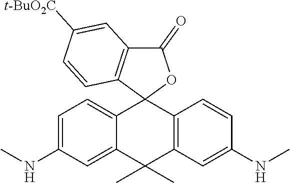

A mixture of Pd₂(dba)₃ (30.2 mg, 0.033 mmol, 0.1 eq.), Xantphos (57.3 mg, 0.099 mmol, 0.3 eq.), Cs₂CO₃ (322.5 mg, 0.99 mmol, 3 eq.) and corresponding triflate 12 (240 mg, 0.33 mmol, 1 eq) in dry 1,4-dioxane (2.5 mL) was degassed on a Schlenk line. Then solution of tert-butyl N-methylcarbamate (108 mg, 0.825 mmol, 2.5 eq.) in 1,4-dioxane (0.5 mL) were introduced. Reaction mixture was stirred in a septa sealed tube at 100° C. under argon for 18 h. Upon cooling, the resulting brown mixture was diluted with water (30 mL), pH adjusted to 5-6 with AcOH extracted with ethyl acetate (3×30 mL), the combined organic layers were washed with brine and dried over Na₂SO₄. Product was isolated by flash column chromatography Büchi Reveleris HP silica 24 g; gradient 5% to 50% EtOAc-Hexane. The fractions containing the product were evaporated, the residue was redissolved in acetonitrile-water (1:1), microfiltered through a 0.45 μm PTFE membrane filter and lyophilized to obtain 173 mg (76%) of violet powder.

¹H NMR (400 MHz, cdcl₃) δ 7.75 (dd, J=7.6, 0.9 Hz, 1H), 7.59 (t, J=7.6 Hz, 1H), 7.52 (d, J=2.2 Hz, 2H), 7.06 (dd, J=7.6, 0.9 Hz, 1H), 7.01 (dd, J=8.6, 2.2 Hz, 2H), 6.76 (d, J=8.6 Hz, 2H), 3.27 (s, 6H), 1.85 (s, 3H), 1.76 (s, 3H), 1.70 (s, 9H), 1.46 (s, 18H).

¹³C NMR (101 MHz, cdcl₃) δ 167.4, 165.5, 156.2, 154.6, 145.3, 144.7, 134.5, 133.4, 129.5, 128.4, 127.9, 126.0, 123.6, 123.6, 123.2, 84.4, 83.6, 80.8, 38.3, 37.3, 35.0, 33.3, 28.5, 28.2.

ESI-MS, positive mode: m/z=685.4 [M+H]⁺.

HRMS (ESI) calcd for C₄₀H₄₉N₂O₈ [M+H]⁺ 685.3483, found 685.3474.

### A.13 Tert-butyl 3,6-bis(dimethylamino)-10,10-dimethyl-3′-oxo-3′H,10H-spiro[anthracene-9,1′-isobenzofuran]-4′-carboxylate (16)

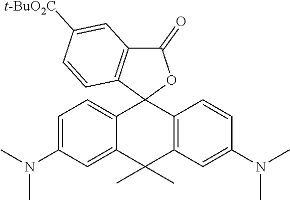

A mixture of Pd₂(dba)₃ (33 mg, 0.036 mmol, 0.1 eq.), Xantphos (62.5 mg, 0.108 mmol, 0.3 eq.), Cs₂CO₃ (352 mg, 1.08 mmol, 3 eq.) and corresponding triflate 12 (250 mg, 0.36 mmol, 1 eq) in dry 1,4-dioxane (2.5 mL) was degassed on a Schlenk line. Then 2M solution of dimethylamine in THF (450 μL, 0.9 mmol, 2.5 eq.) were introduced. Reaction mixture was stirred in a septa sealed tube at 100° C. under argon for 18 h. Upon cooling, the resulting brown mixture was diluted with water (30 mL), pH adjusted to 5-6 with AcOH and extracted with ethyl acetate (3×30 mL), the combined organic layers were washed with brine and dried over Na₂SO₄. Product was isolated by flash column chromatography Büchi Reveleris HP silica 24 g; gradient 20% to 80% EtOAc-Hexane. The fractions containing the product were evaporated, the residue was redissolved in 1,4-dioxane-water (1:1), microfiltered through a 0.45 μm PTFE membrane filter and lyophilized to obtain 131 mg (71%) of violet powder.

¹H NMR (400 MHz, cdcl₃) δ 7.69 (dd, J=7.6, 1.0 Hz, 1H), 7.55 (t, J=7.6 Hz, 1H), 7.09 (dd, J=7.6, 1.0 Hz, 1H), 6.88 (d, J=2.6 Hz, 2H), 6.65 (d, J=8.8 Hz, 2H), 6.53 (dd, J=8.8, 2.6 Hz, 2H), 2.99 (s, 12H), 1.87 (s, 3H), 1.78 (s, 3H), 1.70 (s, 9H).

¹³C NMR (101 MHz, cdcl₃) δ 167.9, 166.0, 157.0, 150.8, 146.7, 134.0, 133.0, 129.1, 128.8, 126.1, 124.0, 119.5, 111.8, 109.2, 86.5, 83.3, 40.6, 38.6, 35.6, 33.1, 28.2.

ESI-MS, positive mode: m/z=513.6 [M+H]⁺.

HRMS (ESI) calcd for C₃₂H₃₇N₂O₄ [M+H]⁺ 513.2748, found 513.2748.

### A.14 Tert-butyl 3,7-bis(dimethylamino)-5,5-dimethyl-3′-oxo-3′H,5H-spiro[dibenzo[b,e]siline-10,1′-isobenzofuran]-4′-carboxylate (17)

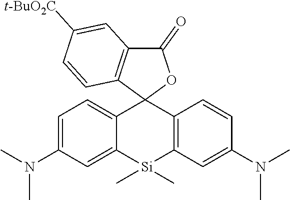

A mixture of Pd₂(dba)₃ (26 mg, 0.0284 mmol, 0.1 eq.), Xantphos (49.3 mg, 0.0852 mmol, 0.3 eq.), Cs₂CO₃ (278 mg, 0.85 mmol, 3 eq.) and corresponding triflate 13 (210 mg, 0.284 mmol, 1 eq) in dry 1,4-dioxane (2.5 mL) was degassed on a Schlenk line. Then 2M solution of dimethylamine in THF (355 μL, 0.71 mmol, 2.5 eq.) were introduced. Reaction mixture was stirred in a septa sealed tube at 100° C. under argon for 18 h. Upon cooling, the resulting brown mixture was diluted with water (30 mL), pH adjusted to 5-6 with AcOH and extracted with ethyl acetate (3×30 mL), the combined organic layers were washed with brine and dried over Na₂SO₄.

Product was isolated by flash column chromatography Büchi Reveleris HP silica 24 g; gradient 5% to 50% EtOAc-Hexane. The fractions containing the product were evaporated, the residue was redissolved in 1,4-dioxane-water (1:1), microfiltered through a 0.45 μm PTFE membrane filter and lyophilized to obtain 73 mg (48%) of light-blue powder.

¹H NMR (400 MHz, cdcl₃) δ 7.69 (dd, J=7.6, 1.0 Hz, 1H), 7.61 (t, J=7.6 Hz, 1H), 7.32 (dd, J=7.6, 1.0 Hz, 1H), 6.96 (d, J=2.9 Hz, 2H), 6.82 (d, J=8.9 Hz, 2H), 6.57 (dd, J=8.9, 2.9 Hz, 2H), 2.96 (s, 12H), 1.67 (s, 9H), 0.64 (s, 3H), 0.60 (s, 3H).

¹³C NMR (101 MHz, cdcl₃) δ 167.8, 166.0, 155.8, 149.4, 136.7, 133.8, 133.3, 131.8, 128.7, 128.3, 126.5, 123.8, 116.6, 113.6, 90.3, 83.3, 40.4, 28.1, 0.4, −1.1.

ESI-MS, positive mode: m/z=529.3 [M+H]⁺.

HRMS (ESI) calcd for C₃₁H₃₇N₂O₄Si [M+H]⁺ 529.2517, found 529.2521.

### A.15 4-TMR-COOH (18)

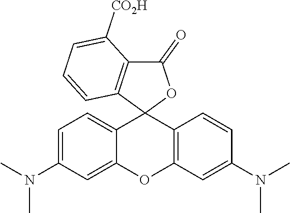

Trifluoroacetic acid (2 mL) was added dropwise to a solution of compound 14 (67 mg, 0.138 mmol) in DCM (8 mL). The resulting coloured solution was stirred at room temperature for 2 h. The reaction mixture was then evaporated to dryness, the residue was re-evaporated three times with toluene to remove excess of trifluoroacetic acid. The residue was lyophilized from 1,4-dioxane-water (1:1). Obtained 74 mg (98%) of pink powder as trifluoroacetic acid salt.

¹H NMR (400 MHz, cd₃od) δ 8.25 (dd, J=7.8, 1.2 Hz, 1H), 7.82 (t, J=7.8 Hz, 1H), 7.60 (dd, J=7.8, 1.2 Hz, 1H), 7.21 (d, J=9.5 Hz, 2H), 7.08 (dd, J=9.5, 2.4 Hz, 2H), 6.87 (d, J=2.4 Hz, 2H), 3.29 (s, 12H).

¹³C NMR (101 MHz, cd₃od) δ 170.5, 168.6, 159.0, 159.0, 157.0, 136.8, 134.1, 132.8, 132.5, 132.2, 132.0, 131.0, 115.5, 115.2, 97.4, 41.0.

ESI-MS, positive mode: m/z=431.2 [M+H]⁺.

HRMS (ESI) calcd for C₂₅H₂₃N₂O₅ [M+H]⁺ 431.1601, found 431.1606.

### A.16 4-580CP-COOH (19)

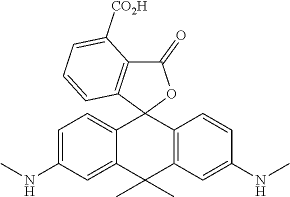

Trifluoroacetic acid (2 mL) was added dropwise to a solution of compound 15 (220 mg, 0.321 mmol) in DCM (8 mL). The resulting coloured solution was stirred at room temperature for 3 h. The reaction mixture was then evaporated to dryness, the residue was re-evaporated three times with toluene to remove excess of trifluoroacetic acid. The residue was lyophilized from 1,4-dioxane-water (1:1). Obtained 170 mg (98%) of violet powder as trifluoroacetic acid salt.

¹H NMR (400 MHz, cd₃od) δ 8.17 (dd, J=7.7, 1.1 Hz, 1H), 7.76 (t, J=7.7 Hz, 1H), 7.52 (dd, J=7.7, 1.1 Hz, 1H), 7.11 (d, J=2.2 Hz, 2H), 7.03 (d, J=9.2 Hz, 2H), 6.64 (dd, J=9.2, 2.2 Hz, 2H), 3.06 (s, 6H), 1.81 (s, 3H), 1.68 (s, 3H).

¹³C NMR (101 MHz, cd₃od) δ 170.6, 168.9, 162.5, 159.4, 139.6, 139.5, 136.6, 136.0, 134.2, 131.72, 131.68, 130.57, 122.1, 112.7 (visible in HSQC spectra), 42.7, 35.8, 31.9, 30.1.

ESI-MS, positive mode: m/z=429.2 [M+H]⁺.

HRMS (ESI) calcd for C₂₆H₂₅N₂O₄ [M+H]⁺ 429.1809, found 429.1807.

### A.17 4-610CP-COOH (20)

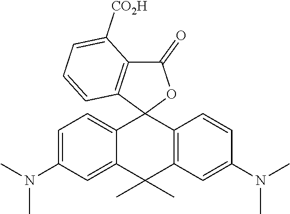

Trifluoroacetic acid (2 mL) was added dropwise to a solution of compound 16 (230 mg, 0.448 mmol) in DCM (8 mL). The resulting coloured solution was stirred at room temperature for 1 h. The reaction mixture was then evaporated to dryness, the residue was re-evaporated three times with toluene to remove excess of trifluoroacetic acid. The residue was lyophilized from 1,4-dioxane-water (1:1). Obtained 251 mg (98%) of dark-violet powder as trifluoroacetic acid salt.

¹H NMR (400 MHz, cd₃od) δ 8.17 (dd, J=7.8, 1.2 Hz, 1H), 7.77 (t, J=7.8 Hz, 1H), 7.53 (dd, J=7.8, 1.2 Hz, 1H), 7.23 (d, J=2.5 Hz, 2H), 7.09 (d, J=9.4 Hz, 2H), 6.84 (dd, J=9.4, 2.5 Hz, 2H), 3.34 (s, 12H), 1.86 (s, 3H), 1.72 (s, 3H).

¹³C NMR (101 MHz, cd₃od) δ 170.6, 168.9, 158.3, 157.9, 138.9, 136.6, 135.9, 134.2, 131.8, 131.7, 130.6, 122.0, 114.0, 112.1, 43.2, 41.0, 36.2, 32.3.

ESI-MS, positive mode: m/z=457.2 [M+H]⁺.

HRMS (ESI) calcd for C₂₈H₂₉N₂O₄ [M+H]⁺ 457.2122, found 457.2119.

### A.18 4-SiR—COOH (21)

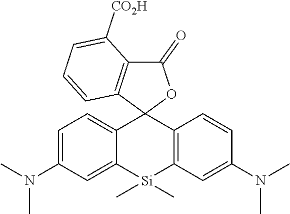

Trifluoroacetic acid (2 mL) was added dropwise to a solution of compound 17 (50 mg, 0.095 mmol) in DCM (8 mL). The resulting coloured solution was stirred at room temperature for 1 h. The reaction mixture was then evaporated to dryness, the residue was re-evaporated three times with toluene to remove excess of trifluoroacetic acid. The residue was lyophilized from 1,4-dioxane-water (1:1). Obtained 54 mg (98%) of blue powder as trifluoroacetic acid salt.

¹H NMR (400 MHz, d₅-pyridine) δ 8.17 (dd, J=7.7, 0.9 Hz, 1H), 7.77 (t, J=7.7 Hz, 1H), 7.54 (dd, J=7.7, 0.9 Hz, 1H), 7.18 (d, J=2.9 Hz, 2H), 7.04 (d, J=9.0 Hz, 2H), 6.54 (dd, J=9.0, 2.9 Hz, 2H), 2.85 (s, 12H), 0.73 (s, 3H), 0.65 (s, 3H).

¹³C NMR (101 MHz, d₅-pyridine) δ 170.1, 169.4, 150.4 (overlapped with pyridine, visible in HMBC spectra), 157.1, 137.4, 135.6, 134.9, 132.3, 129.7, 129.2, 127.0, 124.2 (overlapped with pyridine, visible in HMBC spectra), 117.4, 114.6, 92.3, 40.4, 0.8, −0.8.

ESI-MS, positive mode: m/z=473.2 [M+H]⁺.

HRMS (ESI) calcd for C₂₇H₂₉N₂O₄Si [M+H]⁺ 473.1891, found 473.1890.

### Synthesis Example B

### Synthesis of Rhodamine 4′-Isomerfluorescent Probes

### B.1 DTX-C8-NHBOC (22)

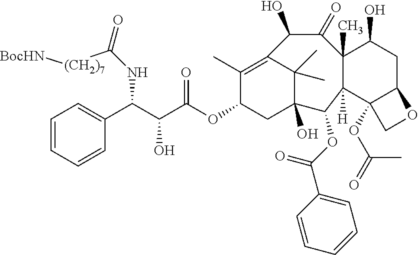

A solution of docetaxel (194 mg, 0.24 mmol) in 95% formic acid (2 mL) was stirred at room temperature for 4 h. Reaction progress was monitored by HPLC analysis. Once reaction was complete, formic acid was evaporated on rotary evaporator and residue was dissolved in water and lyophilised to obtain white powder.

Into a solution of Boc-8-aminooctanoic acid (1.4 eq., 0.336 mmol, 91.7 mg) in MeCN (2 mL) was added HBTU (1.2 eq., 0.288 mmol, 109 mg), followed by DIPEA (4.3 eq., 1 mmol, 100 μL). Mixture was stirred at rt for 5 min and previously obtained des-Boc-docetaxel (0.24 mmol) was added. Mixture was stirred for 1 hour then solvent was removed by rotary evaporator. The residue was re-dissolved in 70% MeCN—H2O mixture, microfiltered through a 0.45 μm PTFE membrane filter and purified by the preparative HPLC (preparative column: Knauer 100 C18, 5 μm, 250×30 mm; solvent A: acetonitrile, solvent B: H2O+0.2% v/v HCOOH; temperature 25° C., gradient A:B—5 min 50:50 isocratic, 5-30 min 50:50 to 100:0 gradient). Fractions containing the product were collected, solvent was removed and obtained residue was lyophilised from 50:50 MeCN:H2O mixture to obtain product as white solid. Obtained 52% 118 mg of white fluffy solid.

¹H NMR (400 MHz, d₆-dmso) δ 8.36 (d, J=9.0 Hz, 1H), 7.99 (dt, J=7.0, 1.4 Hz, 2H), 7.69 (tt, J=7.4, 1.4 Hz, 1H), 7.60 (t, J=7.5 Hz, 2H), 7.39-7.29 (m, 4H), 7.21 (tt, J=7.2, 1.5 Hz, 1H), 6.73 (t, J=5.7 Hz, 1H), 5.95-5.88 (m, 2H), 5.42 (d, J=7.2 Hz, 1H), 5.27 (dd, J=9.0, 5.9 Hz, 1H), 5.10 (s, 1H), 5.01 (d, J=7.2 Hz, 1H), 4.97-4.88 (m, 2H), 4.58 (s, 1H), 4.41 (t, J=6.3 Hz, 1H), 4.09-3.99 (m, 3H), 3.69 (d, J=7.2 Hz, 1H), 2.87 (q, J=6.7 Hz, 2H), 2.28 (s, 1H), 2.24 (s, 3H), 2.15 (t, J=7.3 Hz, 2H), 1.98 (dd, J=15.3, 9.2 Hz, 1H), 1.82 (dd, J=15.4, 8.9 Hz, 1H), 1.75 (s, 3H), 1.73-1.59 (m, 1H), 1.53 (s, 3H), 1.49-1.43 (m, 2H), 1.36 (s, 9H), 1.34-1.26 (m, 2H), 1.18 (q, J=9.0, 7.1 Hz, 6H), 1.03 (s, 3H), 0.99 (s, 3H).

¹³C NMR (101 MHz, d₆-dmso) δ 209.3, 172.6, 171.9, 169.7, 165.3, 155.5, 139.7, 136.8, 135.9, 133.3, 130.0, 129.6, 128.7, 128.1, 127.14, 127.10, 83.7, 80.3, 77.3, 76.9, 75.5, 74.8, 73.8, 73.6, 70.8, 70.0, 57.0, 55.0, 46.0, 42.9, 39.8, 36.5, 35.4, 35.1, 29.5, 28.6, 28.5, 28.3, 26.5, 26.2, 25.4, 22.4, 20.8, 13.7, 9.8.

ESI-MS, positive mode: m/z=949.5 [M+H]⁺.

HRMS (ESI) calcd for C₅₁H₆₉N₂O₁₅ [M+H]⁺ 949.4692, found 949.4683.

### B.2 CTX-C8-NHBOC (23)

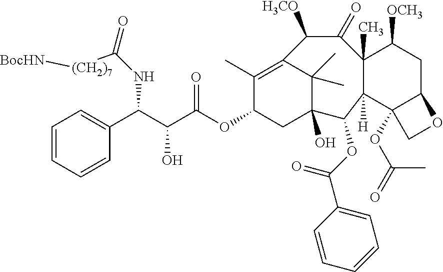

A solution of cabazitaxel (200 mg, 0.24 mmol) in 95% formic acid (2 mL) was stirred at room temperature for 2 h. Reaction progress was monitored by HPLC analysis. Once reaction was complete, formic acid was evaporated on rotary evaporator and residue was dissolved in water and lyophilised to obtain white powder.

Into a solution of Boc-8-aminooctanoic acid (91.7 mg, 0.336 mmol, 1.4 eq.,) in MeCN (2 mL) was added HBTU (109 mg, 0.288 mmol, 1.2 eq.), followed by DIPEA (100 μL, 1 mmol, 4.3 eq.). Mixture was stirred at rt for 5 min and previously obtained des-Boc-docetaxel (0.24 mmol) was added. Mixture was stirred for 1 hour then solvent was removed by rotary evaporator. The residue was re-dissolved in 70% MeCN—H2O mixture, microfiltered through a 0.45 μm PTFE membrane filter and purified by the preparative HPLC (preparative column: Knauer 100 C18, 5 μm, 250×30 mm; solvent A: acetonitrile, solvent B: H2O+0.2% v/v HCOOH; temperature 25° C., gradient A:B—5 min 50:50 isocratic, 5-30 min 50:50 to 100:0 gradient). Fractions containing the product were collected, solvent was removed and obtained residue was lyophilised from 50:50 MeCN:H2O mixture to obtain product as white solid. Obtained 69% (162 mg) of white fluffy solid.

¹H NMR (400 MHz, d₆-dmso) δ 8.37 (d, J=9.1 Hz, 1H), 8.00 (dt, J=7.1, 1.4 Hz, 2H), 7.70 (tt, J=7.4, 2.2 Hz, 1H), 7.61 (t, J=7.4 Hz, 2H), 7.40-7.30 (m, 4H), 7.23 (tt, J=7.4, 1.4 Hz, 1H), 6.74 (t, J=5.7 Hz, 1H), 5.99-5.91 (m, 2H), 5.40 (d, J=7.1 Hz, 1H), 5.30 (dd, J=9.2, 5.8 Hz, 1H), 4.97 (dd, J=9.7, 2.0 Hz, 1H), 4.72 (s, 1H), 4.66 (s, 1H), 4.44 (dd, J=6.8, 5.8 Hz, 1H), 4.04 (s, 2H), 3.77 (dd, J=10.6, 6.5 Hz, 1H), 3.65 (d, J=7.1 Hz, 1H), 3.32 (s, 3H), 3.23 (s, 3H), 2.87 (q, J=6.6 Hz, 2H), 2.72-2.63 (m, 1H), 2.26 (s, 3H), 2.17 (t, J=7.2 Hz, 2H), 1.99 (dd, J=15.4, 9.1 Hz, 1H), 1.92-1.86 (m, 1H), 1.85 (s, 3H), 1.53 (s, 3H), 1.52-1.43 (m, 3H), 1.37 (s, 9H), 1.35-1.30 (m, 2H), 1.24-1.16 (m, 6H), 1.04 (s, 3H), 0.99 (s, 3H).

¹³C NMR (101 MHz, d₆-dmso) δ 204.7, 172.6, 171.9, 169.9, 165.2, 155.5, 139.6, 138.4, 134.9, 133.3, 129.9, 129.6, 128.7, 128.1, 127.1, 83.2, 82.1, 80.3, 80.2, 77.3, 76.9, 75.3, 74.4, 73.6, 70.0, 56.6, 56.6, 56.0, 54.9, 46.4, 43.0, 39.8, 35.4, 34.9, 31.7, 29.5, 28.6, 28.5, 28.3, 26.7, 26.2, 25.4, 22.4, 21.2, 14.0, 10.2.

ESI-MS, positive mode: m/z=977.5 [M+H]⁺.

HRMS (ESI) calcd for C₅₃H₇₃N₂O₁₅ [M+H]⁺ 977.5005, found 977.4995.

### B.3 LTX-C8-NHBOC (24)

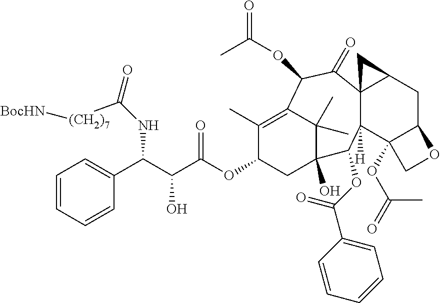

A solution of larotaxel (200 mg, 0.24 mmol) in 95% formic acid (2 mL) was stirred at room temperature for 1 h. Reaction progress was monitored by HPLC analysis. Once reaction was complete, formic acid was evaporated on rotary evaporator and residue was dissolved in water and lyophilised to obtain white powder.

Into a solution of Boc-8-aminooctanoic acid (91.7 mg, 0.336 mmol, 1.4 eq.,) in MeCN (2 mL) was added HBTU (109 mg, 0.288 mmol, 1.2 eq.), followed by DIPEA (100 μL, 1 mmol, 4.3 eq.). Mixture was stirred at rt for 5 min and previously obtained des-Boc-docetaxel (0.24 mmol) was added. Mixture was stirred for 1 hour then solvent was removed by rotary evaporator. The residue was re-dissolved in 70% MeCN—H2O mixture, microfiltered through a 0.45 μm PTFE membrane filter and purified by the preparative HPLC (preparative column: Knauer 100 C18, 5 μm, 250×30 mm; solvent A: acetonitrile, solvent B: H2O+0.2% v/v HCOOH; temperature 25° C., gradient A:B—5 min 50:50 isocratic, 5-30 min 50:50 to 100:0 gradient). Fractions containing the product were collected, solvent was removed and obtained residue was lyophilised from 50:50 MeCN:H2O mixture to obtain product as white solid.

Obtained 57% (133 mg) of white fluffy solid.

¹H NMR (400 MHz, d₆-dmso) δ 8.34 (d, J=9.2 Hz, 1H), 8.08-8.00 (m, 2H), 7.69 (tt, J=7.4, 1.4 Hz, 1H), 7.61 (t, J=7.5 Hz, 2H), 7.41-7.29 (m, 4H), 7.19 (tt, J=7.4, 1.7 Hz, 1H), 6.73 (t, J=5.7 Hz, 1H), 6.12 (s, 1H), 6.02-5.91 (m, 1H), 5.87 (d, J=6.8 Hz, 1H), 5.44 (d, J=7.7 Hz, 1H), 5.34 (dd, J=9.2, 5.2 Hz, 1H), 4.77 (s, 1H), 4.72 (d, J=3.0 Hz, 1H), 4.49 (dd, J=6.8, 5.2 Hz, 1H), 4.10-3.94 (m, 2H), 3.91 (d, J=7.6 Hz, 1H), 2.86 (q, J=6.5 Hz, 2H), 2.36-2.23 (m, 4H), 2.17-1.87 (m, 9H), 1.74 (d, J=1.4 Hz, 3H), 1.54 (dd, J=7.3, 4.8 Hz, 1H), 1.50-1.41 (m, 2H), 1.36 (s, 9H), 1.35-1.28 (m, 2H), 1.19-1.14 (m, 6H), 1.12 (s, 3H), 1.08 (s, 3H).

¹³C NMR (101 MHz, d₆-dmso) δ 202.0, 172.8, 172.4, 170.2, 169.5, 165.8, 156.0, 140.5, 140.3, 134.0, 133.7, 130.4, 130.1, 129.2, 128.6, 127.5, 127.5, 84.2, 80.0, 79.0, 77.8, 77.7, 75.7, 74.9, 74.0, 70.0, 55.1, 43.0, 40.3, 38.0, 36.1, 35.8, 34.9, 31.7, 29.9, 29.1, 28.9, 28.7, 26.7, 26.4, 26.0, 25.8, 22.4, 21.8, 21.0, 15.2, 14.4.

ESI-MS, positive mode: m/z=973.6 [M+H]⁺.

HRMS (ESI) calcd for C₅₃H₆₉N₂O₁₅ [M+H]⁺ 973.4692, found 973.4687.

### B.4 4-TMR-DTX (25)

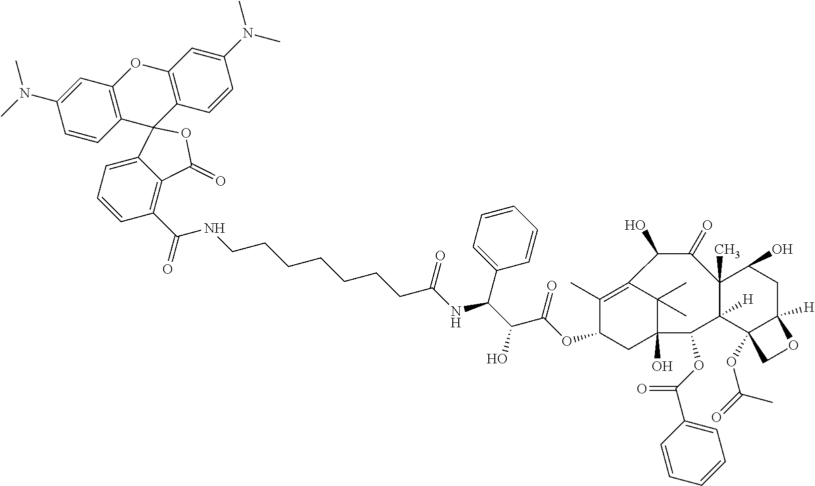

A solution of DTX-C8-NHBoc (22) (14.2 mg, 0.015 mmol) in 95% formic acid (1 mL) was stirred at room temperature for 1 h. Reaction progress was monitored by HPLC analysis. Once reaction was complete, formic acid was evaporated on rotary evaporator and residue was dissolved in water and lyophilised to obtain white powder, which was used further without any additional purifications.

The 4-TMR-COOH (18) dye TFA salt (5.5 mg, 0.01 mmol, 1 eq), DIPEA (52 μL, 0.3 mmol, 30 eq.) and HBTU (4.5 mg, 0.012 mmol, 1.2 eq.) were dissolved in 400 μL of dry MeCN and stirred at room temperature for 5 min. A solution of previously obtained deprotected DTX-C8-NH2 derivative (0.015 mmol, 1.5 eq) in MeCN and 10 μL of DIPEA were added to the reaction mixture and stirring continued for 1 hour. Reaction was monitored by HPLC analysis. Purified by preparative HPLC (preparative column: Eurospher 100 C18, 5 μm, 250×20 mm; solvent A: acetonitrile, solvent B: H2O+0.2% v/v HCOOH; temperature 25° C., gradient A:B—5 min 20:80 isocratic, 5-30 min 20:80 to 70:30 gradient). Additionally purified by flash column chromatography (Interchim Puriflash 12 g, 15 μm normal phase column, gradient 3% to 30% DCM-MeOH), microfiltered through a 0.45 μm PTFE membrane filter and lyophilized from acetonitrile: water mixture. Yield 26% (3.3 mg) of light purple fluffy solid.

¹H NMR (400 MHz, d₆-dmso) δ 9.06 (t, J=5.5 Hz, 1H), 8.38 (d, J=9.0 Hz, 1H), 8.02-7.95 (m, 2H), 7.84 (dd, J=7.6, 1.1 Hz, 1H), 7.76 (t, J=7.6 Hz, 1H), 7.67 (tt, J=7.2, 1.3 Hz, 1H), 7.64-7.55 (m, 2H), 7.41-7.28 (m, 4H), 7.25-7.17 (m, 2H), 6.64-6.57 (m, 2H), 6.53-6.43 (m, 4H), 5.99-5.86 (m, 2H), 5.41 (d, J=7.2 Hz, 1H), 5.27 (dd, J=9.0, 5.9 Hz, 1H), 5.09 (d, J=2.6 Hz, 1H), 5.01 (d, J=7.3 Hz, 1H), 4.94 (d, J=2.5 Hz, 1H), 4.90 (dd, J=9.6, 2.2 Hz, 1H), 4.58 (s, 1H), 4.41 (t, J=6.4 Hz, 1H), 4.12-3.96 (m, 3H), 3.68 (d, J=7.1 Hz, 1H), 3.31-3.27 (m, 2H, overlapped with HDO), 2.94 (s, 13H), 2.33-2.26 (m, 1H), 2.23 (s, 3H), 2.17 (t, J=7.3 Hz, 2H), 2.00-1.94 (m, 1H), 1.86-1.78 (m, 1H), 1.75 (s, 3H), 1.69-1.60 (m, 1H), 1.57-1.46 (m, 7H), 1.35-1.25 (m, 6H), 1.03 (s, 3H), 0.98 (s, 3H).

¹H-¹³C NMR ((400, 101) MHz, d₆-dmso) δ (7.95 129.99), (7.81 130.12), (7.73 135.55), (7.64 133.72), (7.56 129.09), (7.33 128.55), (7.28 127.56), (7.20 125.80), (7.16 127.51), (6.57 129.08), (6.46 98.37), (6.46 109.29), (5.88 70.41), (5.38 75.24), (5.24 55.43), (5.06 74.19), (4.87 84.14), (4.38 74.03), (4.01 71.22), (3.98 75.85), (3.65 46.37), (3.29 39.79), (2.91 40.22), (2.23, 1.63 36.87), (2.20 22.82), (2.14 35.86), (1.97, 1.78 35.53), (1.71 14.09), (1.53 29.31), (1.49 10.24), (1.47 25.85), (1.31 26.89), (1.21 29.04), (1.21 29.22), (0.99 26.94), (0.95 21.26).

ESI-MS, positive mode: m/z=1261.6 [M+H]⁺.

HRMS (ESI) calcd for C₇₁H₈₁N₄O₁₇ [M+H]⁺ 1261.5591, found 1261.5594.

### B.5 4-TMR-CTX (26)

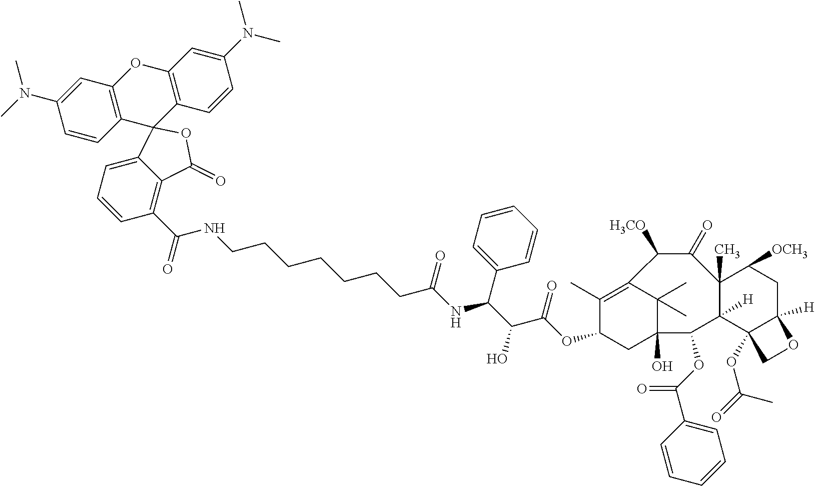

A solution of CTX-C8-NHBoc (23) (14.6 mg, 0.015 mmol) in 95% formic acid (1 mL) was stirred at room temperature for 1 h. Reaction progress was monitored by HPLC analysis. Once reaction was complete, formic acid was evaporated on rotary evaporator and residue was dissolved in water and lyophilised to obtain white powder, which was used further without any additional purifications.

The 4-TMR-COOH (18) dye TFA salt (5.5 mg, 0.01 mmol, 1 eq), DIPEA (52 μL, 0.3 mmol, 30 eq.) and HBTU (4.5 mg, 0.012 mmol, 1.2 eq.) were dissolved in 400 μL of dry MeCN and stirred at room temperature for 5 min. A solution of previously obtained deprotected CTX-C8-NH2 derivative (0.015 mmol, 1.5 eq) in MeCN and 10 μL of DIPEA were added to the reaction mixture and stirring continued for 1 hour. Reaction was monitored by HPLC analysis. Purified by preparative HPLC (preparative column: Eurospher 100 C18, 5 μm, 250×20 mm; solvent A: acetonitrile, solvent B: H2O+0.2% v/v HCOOH; temperature 25° C., gradient A:B—5 min 20:80 isocratic, 5-30 min 20:80 to 70:30 gradient). Additionally purified by flash column chromatography (Interchim Puriflash 12 g, 15 μm column, gradient 3% to 30% DCM-MeOH), microfiltered through a 0.45 μm PTFE membrane filter and lyophilised from acetonitrile: water mixture. Yield 26% (3.3 mg) of light purple fluffy solid.

¹H NMR (400 MHz, d₆-dmso) δ 9.03 (t, J=5.5 Hz, 1H), 8.35 (d, J=9.1 Hz, 1H), 7.95 (d, J=7.0 Hz, 2H), 7.81 (dd, J=7.5, 1.1 Hz, 1H), 7.73 (t, J=7.6 Hz, 1H), 7.67-7.61 (m, 1H), 7.59-7.53 (m, 2H), 7.36-7.28 (m, 4H), 7.22-7.15 (m, 2H), 6.58 (dd, J=9.4, 1.6 Hz, 2H), 6.50-6.42 (m, 4H), 5.97-5.87 (m, 2H), 5.36 (d, J=7.0 Hz, 1H), 5.27 (dd, J=6.9, 2.4 Hz, 1H), 4.92 (d, J=9.1 Hz, 1H), 4.67 (s, 1H), 4.62 (s, 1H), 4.40 (dd, J=6.9, 5.8 Hz, 1H), 3.99 (s, 2H), 3.72 (dd, J=10.9, 6.4 Hz, 1H), 3.60 (d, J=7.1 Hz, 1H), 3.28-3.25 (m, 5H), 3.18 (s, 3H), 2.91 (s, 12H), 2.66-2.58 (m, 1H), 2.22 (s, 3H), 2.15 (t, J=7.8 Hz, 2H), 1.99-1.92 (m, 3H), 1.88-1.81 (m, 1H), 1.80 (s, 3H), 1.54-1.47 (m, 8H), 1.27-1.23 (m, 6H), 1.00 (s, 3H), 0.94 (s, 3H).

¹H-¹³C NMR ((400, 101) MHz, d₆-dmso) δ (7.95 130.02), (7.80 130.16), (7.74 135.58), (7.64 133.77), (7.56 129.10), (7.32 128.58), (7.30 127.56), (7.20 125.78), (7.18 127.53), (6.57 129.08), (6.46 109.29), (6.46 98.37), (5.91 70.40), (5.35 74.79), (5.26 55.39), (4.93 83.66), (4.67 82.52), (4.39 74.01), (3.99 75.71), (3.72 80.68), (3.60 46.84), (3.29 39.78), (3.27 57.02), (3.18 57.08), (2.91 40.22), (2.63, 1.46 32.13), (2.21 22.82), (2.15 35.84), (1.98, 1.83 35.48), (1.95 26.99), (1.80 14.44), (1.49 10.56), (1.47 25.85), (1.32 26.90), (1.21 29.21), (1.20 31.71), (1.00 27.12), (0.94 21.63).

ESI-MS, positive mode: m/z=1289.6 [M+H]⁺.

HRMS (ESI) calcd for C₇₃H₈₅N₄O₁₇ [M+H]⁺ 1289.5904, found 1289.5919.

### B.6 4-TMR-LTX (27)

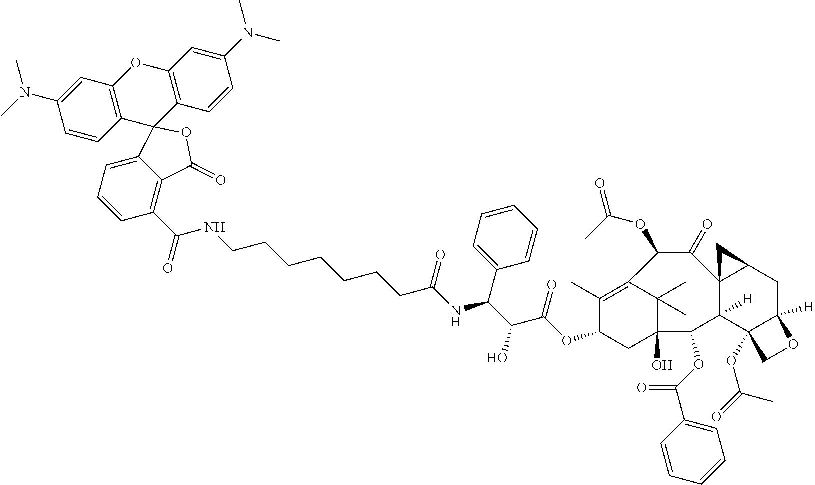

A solution of LTX-C8-NHBoc (23) (14.6 mg, 0.015 mmol) in 95% formic acid (1 mL) was stirred at room temperature for 1 h. Reaction progress was monitored by H PLC analysis. Once reaction was complete, formic acid was evaporated on rotary evaporator and residue was dissolved in water and lyophilised to obtain white powder, which was used further without any additional purifications.

The 4-TMR-COOH (18) dye TFA salt (5.5 mg, 0.01 mmol, 1 eq), DIPEA (52 μL, 0.3 mmol, 30 eq.) and HBTU (4.5 mg, 0.012 mmol, 1.2 eq.) were dissolved in 400 μL of dry MeCN and stirred at room temperature for 5 min. A solution of previously obtained deprotected LTX-C8-NH2 derivative (0.015 mmol, 1.5 eq) in MeCN and 10 μL of DIPEA were added to the reaction mixture and stirring continued for 1 hour. Reaction was monitored by HPLC analysis.

Purified by preparative HPLC (preparative column: Eurospher 100 C18, 5 μm, 250×20 mm; solvent A: acetonitrile, solvent B: H2O+0.2% v/v HCOOH; temperature 25° C., gradient A:B—5 min 20:80 isocratic, 5-30 min 20:80 to 70:30 gradient). Additionally purified by flash column chromatography (Interchim Puriflash 12 g, 15 μm column, gradient 3% to 30% DCM-MeOH), microfiltered through a 0.45 μm PTFE membrane filter and lyophilised from acetonitrile: water mixture. Yield 44% (5.6 mg) of light purple fluffy solid.

¹H NMR (400 MHz, d₆-dmso) δ 9.07 (t, J=5.5 Hz, 1H), 8.50 (d, J=9.1 Hz, 1H), 8.02 (d, J=7.2 Hz, 2H), 7.84 (d, J=7.5 Hz, 1H), 7.76 (t, J=7.6 Hz, 1H), 7.68 (dd, J=8.4, 6.1 Hz, 1H), 7.60 (t, J=7.6 Hz, 2H), 7.37-7.30 (m, 4H), 7.23 (d, J=7.5 Hz, 1H), 7.17 (tt, J=7.3, 1.9 Hz, 1H), 6.61 (d, J=9.5 Hz, 2H), 6.50-6.46 (m, 4H), 6.12 (s, 1H), 5.94 (t, J=9.0 Hz, 1H), 5.50-5.33 (m, 2H), 5.32 (dd, J=9.1, 5.4 Hz, 1H), 4.77 (s, 1H), 4.71 (d, J=3.1 Hz, 1H), 4.49 (d, J=5.5 Hz, 1H), 4.05-3.96 (m, 2H), 3.90 (d, J=7.6 Hz, 1H), 3.30-3.27 (m, 2H), 2.94 (s, 12H), 2.32-2.25 (m, 4H), 2.17-1.86 (m, 9H), 1.74 (s, 3H), 1.59-1.44 (m, 5H), 1.35-1.20 (m, 7H), 1.11 (s, 3H), 1.08 (s, 3H).

¹H-¹³C NMR ((400, 101) MHz, d₆-dmso) δ (8.00 130.09), (7.81 130.12), (7.74 135.60), (7.65 133.75), (7.57 129.19), (7.31 128.62), (7.30 127.50), (7.20 125.77), (7.15 127.38), (6.57 129.09), (6.47 109.30), (6.46 98.37), (6.09 75.67), (5.92 69.90), (5.40 79.98), (5.29 55.23), (4.69 84.16), (4.46 73.96), (3.96 74.90), (3.88 37.97), (3.29 39.77), (2.91 38.96), (2.91 40.22), (2.26 22.36), (2.25 26.03), (2.13 35.83), (2.09 20.99), (1.81 14.82), (1.71 14.39), (1.52 29.37), (1.45 25.88), (1.30 26.91), (1.27 29.01), (1.21 29.10), (1.15 31.63), (1.08 21.82), (1.05 26.34).

ESI-MS, positive mode: m/z=1285.7 [M+H]⁺.

HRMS (ESI) calcd for C₇₃H₈₁N₄O₁₇ [M+H]⁺ 1285.5591, found 1285.5591.

### B.7 4-580CP-LTX (28)

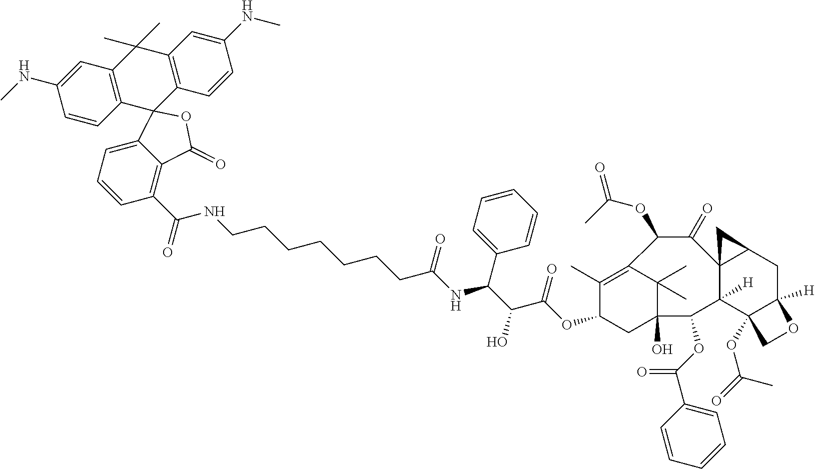

A solution of LTX-C8-NHBoc (23) (14.6 mg, 0.015 mmol) in 95% formic acid (1 mL) was stirred at room temperature for 1 h. Reaction progress was monitored by HPLC analysis. Once reaction was complete, formic acid was evaporated on rotary evaporator and residue was dissolved in water and lyophilised to obtain a white powder, which was used further without any additional purifications.

The 4-580CP-COOH (19) dye TFA salt (5.4 mg, 0.01 mmol, 1 eq), DIPEA (52 μL, 0.3 mmol, 30 eq.) and HBTU (4.5 mg, 0.012 mmol, 1.2 eq.) were dissolved in 400 μL of dry MeCN and stirred at room temperature for 5 min. A solution of previously obtained deprotected LTX-C8-NH2 derivative (0.015 mmol, 1.5 eq) in MeCN and 10 μL of DIPEA were added to the reaction mixture and stirring continued for 1 hour. Reaction was monitored by HPLC analysis.

Purified by preparative HPLC (preparative column: Eurospher 100 C18, 5 μm, 250×20 mm; solvent A: acetonitrile, solvent B: H2O+0.2% v/v HCOOH; temperature 25° C., gradient A:B—5 min 30:70 isocratic, 5-30 min 30:70 to 70:30 gradient). Additionally purified by flash column chromatography (Interchim Puriflash 12 g, 15 μm column, gradient 2% to 15% DCM-MeOH) and lyophilised from acetonitrile: water mixture. Yield 38% (4.9 mg) of violet fluffy solid.

¹H NMR (500 MHz, d₆-dmso) δ 9.14 (t, J=5.5 Hz, 1H), 8.37 (d, J=9.2 Hz, 1H), 8.03 (d, J=7.1 Hz, 2H), 7.78 (dd, J=7.5, 1.0 Hz, 1H), 7.71-7.65 (m, 2H), 7.60 (t, J=7.6 Hz, 2H), 7.38-7.31 (m, 4H), 7.17 (tt, J=7.0, 1.6 Hz, 1H), 7.05 (dd, J=7.7, 1.0 Hz, 1H), 6.76 (d, J=2.4 Hz, 2H), 6.45 (d, J=8.6 Hz, 2H), 6.41-6.36 (m, 2H), 6.12 (s, 1H), 5.96 (t, J=8.9 Hz, 1H), 5.91-5.80 (m, 3H), 5.43 (d, J=7.7 Hz, 1H), 5.34 (dd, J=9.2, 5.2 Hz, 1H), 4.78 (s, 1H), 4.72 (d, J=4.1 Hz, 1H), 4.49 (dd, J=6.8, 5.3 Hz, 1H), 4.05-3.97 (m, 2H), 3.91 (d, J=7.6 Hz, 1H), 3.30-3.16 (m, 2H), 2.70 (s, 3H), 2.69 (s, 3H), 2.31-2.25 (m, 4H), 2.18-1.88 (m, 9H), 1.75 (s, 3H), 1.74 (s, 3H), 1.65 (s, 3H), 1.57-1.45 (m, 5H), 1.37-1.23 (m, 7H), 1.11 (s, 3H), 1.08 (s, 3H).

¹H-¹³C NMR ((500, 126) MHz, d₆-dmso) δ (8.03 129.69), (7.77 129.15), (7.69 134.93), (7.68 133.36), (7.60 128.78), (7.35 128.15), (7.33 127.07), (7.17 127.04), (7.06 125.16), (6.75 107.95), (6.46 128.47), (6.38 111.40), (6.11 75.23), (5.95 69.56), (5.44 79.53), (5.34 54.71), (4.71 83.75), (4.49 73.54), (4.02, 3.99 74.38), (3.91 37.53), (3.32 39.34), (2.69 29.56), (2.31, 1.95 25.56), (2.30 21.95), (2.16 35.41), (2.12 20.57), (2.09, 1.89 35.55), (2.03, 1.53 14.88), (1.75 33.25), (1.73 13.98), (1.65 34.56), (1.55 28.92), (1.47 25.46), (1.36 28.30), (1.33 26.48), (1.23 28.88), (1.23 28.57), (1.11 21.41), (1.07 25.91).

ESI-MS, positive mode: m/z=1283.7 [M+H]⁺.

HRMS (ESI) calcd for C₇₄H₈₂N₄O₁₆ [M+H]⁺ 1283.5799, found 1283.5796

### B.8 4-610CP-DTX (29)

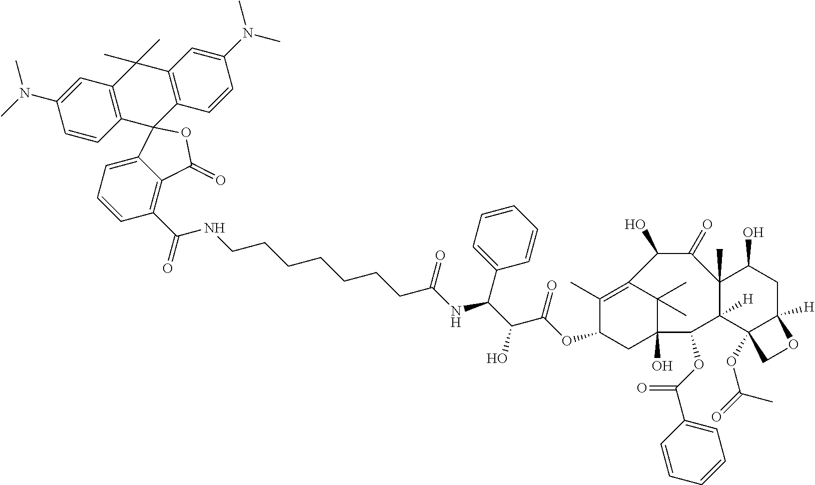

A solution of DTX-C8-NHBoc (22) (14.2 mg, 0.015 mmol) in 95% formic acid (1 mL) was stirred at room temperature for 1 h. Reaction progress was monitored by HPLC analysis. Once reaction was complete, formic acid was evaporated on rotary evaporator and residue was dissolved in water and lyophilised to obtain white powder, which was used further without any additional purifications.

The 4-610CP-COOH (20) dye TFA salt (5.7 mg, 0.01 mmol, 1 eq), DIPEA (52 μL, 0.3 mmol, 30 eq.) and HBTU (4.5 mg, 0.012 mmol, 1.2 eq.) were dissolved in 400 μL of dry MeCN and stirred at room temperature for 5 min. A solution of previously obtained deprotected DTX-C8-NH2 derivative (0.015 mmol, 1.5 eq) in MeCN and 10 μL of DIPEA were added to the reaction mixture and stirring continued for 1 hour. Reaction was monitored by HPLC analysis.

Purified by preparative HPLC (preparative column: Eurospher 100 C18, 5 μm, 250×20 mm; solvent A: acetonitrile, solvent B: H2O+0.2% v/v HCOOH; temperature 25° C., gradient A:B—5 min 30:70 isocratic, 5-30 min 30:70 to 100:0 gradient). Lyophilised from acetonitrile: water mixture. Yield 42% (5.4 mg) of slightly blue fluffy solid.

¹H NMR (400 MHz, d₆-dmso) δ 9.11 (t, J=5.5 Hz, 1H), 8.37 (d, J=9.0 Hz, 1H), 8.03-7.95 (m, 2H), 7.78 (dd, J=7.5, 1.0 Hz, 1H), 7.72-7.64 (m, 2H), 7.59 (t, J=7.4 Hz, 2H), 7.39-7.30 (m, 4H), 7.20 (tt, J=7.1, 1.4 Hz, 1H), 7.04 (d, J=7.7 Hz, 1H), 6.91 (s, 2H), 6.63-6.52 (m, 4H), 6.03-5.82 (m, 2H), 5.41 (d, J=7.3 Hz, 1H), 5.28 (dd, J=9.1, 5.8 Hz, 1H), 5.10 (s, 1H), 5.01 (s, 1H), 4.90 (dd, J=9.8, 1.6 Hz, 1H), 4.58 (s, 1H), 4.41 (t, J=5.0 Hz, 1H), 4.10-3.96 (m, 3H), 3.68 (d, J=7.2 Hz, 1H), 3.31-3.27 (m, 2H), 2.94 (s, 12H), 2.32-2.25 (m, 1H), 2.24 (s, 3H), 2.17 (t, J=7.3 Hz, 2H), 1.97 (dd, J=15.1, 9.6 Hz, 1H), 1.86-1.80 (m, 4H), 1.75 (s, 3H), 1.72 (s, 3H), 1.69-1.63 (m, 1H), 1.61-1.55 (m, 2H), 1.52 (s, 3H), 1.52-1.47 (m, 2H), 1.38-1.25 (m, 6H), 1.03 (s, 3H), 0.98 (s, 3H).

¹H-¹³C NMR ((400, 101) MHz, d₆-dmso) δ (7.95 130.00), (7.74 129.59), (7.65 135.43), (7.64 133.73), (7.56 129.09), (7.33 128.57), (7.29 127.57), (7.17 127.53), (7.01 125.46), (6.88 109.57), (6.54 112.20), (6.53 128.80), (5.88 70.45), (5.38 75.26), (5.25 55.43), (5.06 74.19), (4.86 84.14), (4.38 74.02), (4.02 71.22), (3.99 75.89), (3.65 46.41), (3.30 39.78), (2.90 40.44), (2.24, 1.62 36.90), (2.21 22.82), (2.14 35.86), (1.94, 1.78 35.44), (1.79 34.01), (1.72 14.09), (1.68 34.96), (1.54 29.35), (1.49 10.24), (1.47 25.86), (1.32 26.91), (1.24 29.03), (1.20 29.22), (1.00 26.94), (0.95 21.26).

ESI-MS, positive mode: m/z=1287.6 [M+H]⁺.

HRMS (ESI) calcd for C₇₄H₈₇N₄O₁₆ [M+H]⁺ 1287.6112, found 1287.6110.

### B.9 4-610CP-CTX (30)

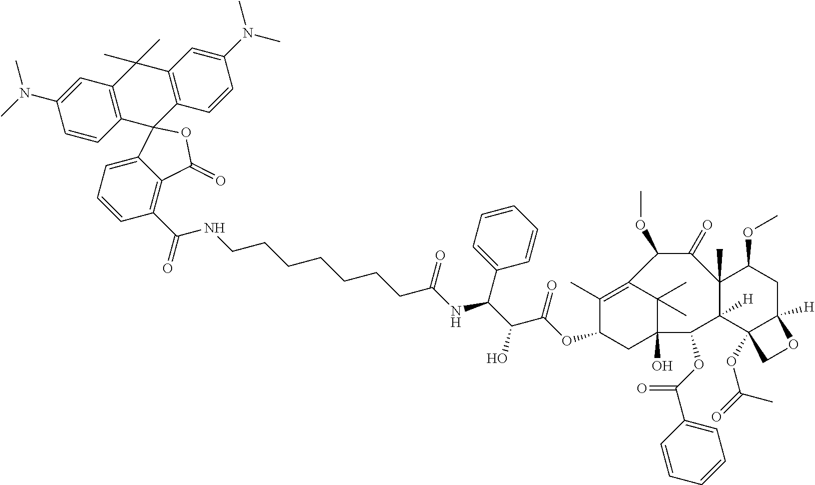

A solution of CTX-C8-NHBoc (23) (14.6 mg, 0.015 mmol) in 95% formic acid (1 mL) was stirred at room temperature for 1 h. Reaction progress was monitored by HPLC analysis. Once reaction was complete, formic acid was evaporated on rotary evaporator and residue was dissolved in water and lyophilised to obtain white powder, which was used further without any additional purifications.

The 4-610CP-COOH (20) dye TFA salt (5.7 mg, 0.01 mmol, 1 eq), DIPEA (52 μL, 0.3 mmol, 30 eq.) and HBTU (4.5 mg, 0.012 mmol, 1.2 eq.) were dissolved in 400 μL of dry MeCN and stirred at room temperature for 5 min. A solution of previously obtained deprotected CTX-C8-NH2 derivative (0.015 mmol, 1.5 eq) in MeCN and 10 μL of DIPEA were added to the reaction mixture and stirring continued for 1 hour. Reaction was monitored by HPLC analysis.

Purified by preparative HPLC (preparative column: Eurospher 100 C18, 5 μm, 250×20 mm; solvent A: acetonitrile, solvent B: H2O+0.2% v/v HCOOH; temperature 25° C., gradient A:B—5 min 30:70 isocratic, 5-30 min 30:70 to 100:0 gradient). Lyophilised from acetonitrile: water mixture.

Yield 46% (6.0 mg) of light blue fluffy solid.

¹H NMR (400 MHz, d₆-dmso) δ 9.09 (t, J=5.5 Hz, 1H), 8.37 (d, J=9.0 Hz, 1H), 7.98-7.91 (m, 2H), 7.75 (dd, J=7.5, 1.0 Hz, 1H), 7.68-7.61 (m, 2H), 7.61-7.51 (m, 2H), 7.36-7.26 (m, 4H), 7.18 (tt, J=7.1, 1.6 Hz, 1H), 7.01 (dd, J=7.7, 1.0 Hz, 1H), 6.88 (d, J=2.0 Hz, 2H), 6.60-6.49 (m, 4H), 6.00-5.87 (m, 2H), 5.36 (d, J=7.1 Hz, 1H), 5.26 (dd, J=9.1, 5.8 Hz, 1H), 4.92 (dd, J=9.6, 2.1 Hz, 1H), 4.68 (s, 1H), 4.62 (s, 1H), 4.40 (t, J=6.2 Hz, 1H), 3.99 (s, 2H), 3.72 (dd, J=10.6, 6.6 Hz, 1H), 3.60 (d, J=7.1 Hz, 1H), 3.34-3.31 (m, 2H), 3.27 (s, 3H), 3.18 (s, 3H), 2.91 (s, 12H), 2.69-2.57 (m, 1H), 2.21 (s, 3H), 2.15 (t, J=7.3 Hz, 2H), 1.97-1.91 (m, 1H), 1.88-1.82 (m, 1H), 1.80 (s, 3H), 1.79 (s, 3H), 1.69 (s, 3H), 1.56-1.43 (m, 8H), 1.37-1.23 (m, 6H), 1.00 (s, 3H), 0.94 (s, 3H).

¹H-¹³C NMR ((400, 101) MHz, d₆-dmso) δ (7.95 130.01), (7.75 129.60), (7.66 135.42), (7.64 133.80), (7.56 129.10), (7.33 128.58), (7.30 127.57), (7.18 127.54), (7.01 125.47), (6.88 109.55), (6.53 112.18), (6.53 128.79), (5.92 70.41), (5.36 74.79), (5.26 55.41), (4.91 83.63), (4.67 82.52), (4.40 74.01), (3.99 75.70), (3.72 80.67), (3.60 46.85), (3.30 39.78), (3.27 57.03), (3.18 57.07), (2.90 40.41), (2.62, 1.46 32.14), (2.21 22.81), (2.15 35.84), (1.94, 1.84 35.26), (1.80 14.44), (1.79 34.01), (1.68 34.94), (1.54 29.33), (1.49 10.58), (1.48 25.84), (1.32 26.90), (1.24 29.05), (1.22 29.13), (1.00 27.12), (0.94 21.63).

ESI-MS, positive mode: m/z=1315.6 [M+H]⁺.

HRMS (ESI) calcd for C76H₉₁N₄₀₁₆ [M+H]⁺ 1315.6425, found 1315.6409.

### B.10 4-610CP-LTX (31)

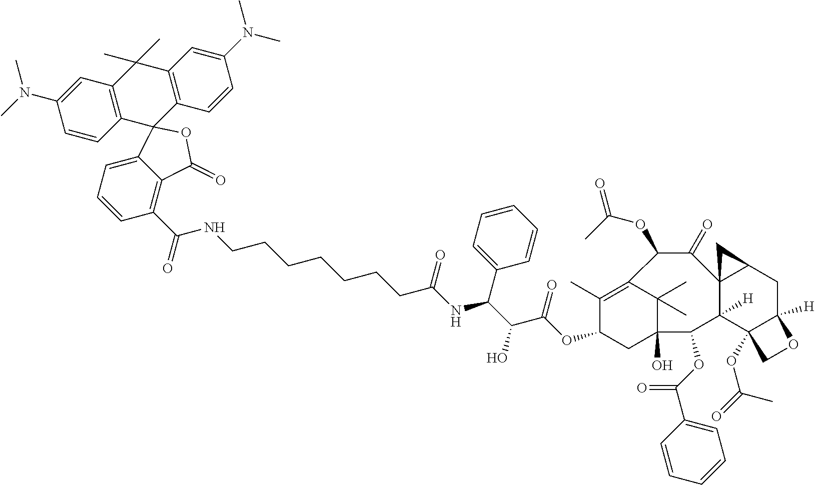

A solution of LTX-C8-NHBoc (23) (14.6 mg, 0.015 mmol) in 95% formic acid (1 mL) was stirred at room temperature for 1 h. Reaction progress was monitored by HPLC analysis. Once reaction was complete, formic acid was evaporated on rotary evaporator and residue was dissolved in water and lyophilised to obtain white powder, which was used further without any additional purifications.

The 4-610CP-COOH (20) dye TFA salt (5.7 mg, 0.01 mmol, 1 eq), DIPEA (52 μL, 0.3 mmol, 30 eq.) and HBTU (4.5 mg, 0.012 mmol, 1.2 eq.) were dissolved in 400 μL of dry MeCN and stirred at room temperature for 5 min. A solution of previously obtained deprotected LTX-C8-NH2 derivative (0.015 mmol, 1.5 eq) in MeCN and 10 μL of DIPEA were added to the reaction mixture and stirring continued for 1 hour. Reaction was monitored by HPLC analysis.

Purified by preparative HPLC (preparative column: Eurospher 100 C18, 5 μm, 250×20 mm; solvent A: acetonitrile, solvent B: H2O+0.2% v/v HCOOH; temperature 25° C., gradient A:B—5 min 30:70 isocratic, 5-30 min 30:70 to 100:0 gradient). Lyophilised from acetonitrile: water mixture.

Yield 35% (4.7 mg) of light blue fluffy solid.

¹H NMR (400 MHz, d₆-dmso) δ 9.09 (t, J=5.5 Hz, 1H), 8.37 (d, J=8.8 Hz, 1H), 8.04-7.96 (m, 2H), 7.75 (d, J=7.4 Hz, 1H), 7.70-7.61 (m, 2H), 7.57 (t, J=7.6 Hz, 2H), 7.37-7.25 (m, 4H), 7.14 (tt, J=7.0, 1.9 Hz, 1H), 7.01 (d, J=7.6 Hz, 1H), 6.88 (d, J=2.1 Hz, 2H), 6.59-6.48 (m, 4H), 6.09 (s, 1H), 6.00-5.81 (m, 2H), 5.40 (d, J=7.6 Hz, 1H), 5.31 (dd, J=9.1, 5.3 Hz, 1H), 4.74 (s, 1H), 4.68 (d, J=3.7 Hz, 1H), 4.46 (d, J=3.8 Hz, 1H), 3.97 (dd, J=20.5, 8.5 Hz, 2H), 3.88 (d, J=7.6 Hz, 1H), 3.28-3.26 (m, 2H), 2.91 (s, 12H), 2.27 (s, 4H), 2.15-1.84 (m, 9H), 1.79 (s, 3H), 1.71 (s, 3H), 1.69 (s, 3H), 1.55-1.44 (m, 5H), 1.33-1.21 (m, 7H), 1.08 (s, 3H), 1.05 (s, 3H).

ESI-MS, positive mode: m/z=1311.6 [M+H]⁺.

HRMS (ESI) calcd for C₇₆H₈₇N₄O₁₆ [M+H]⁺ 1311.6112, found 1311.6085.

### B.11 4-SiR-DTX (32)

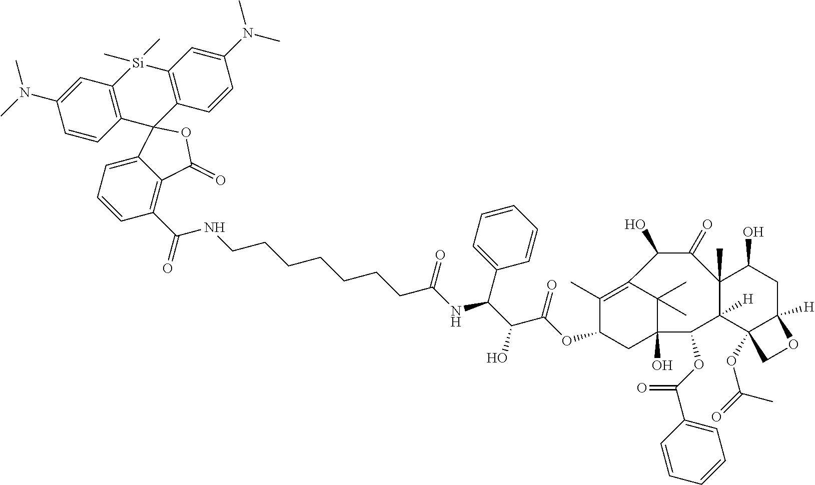

A solution of DTX-C8-NHBoc (22) (14.2 mg, 0.015 mmol) in 95% formic acid (1 mL) was stirred at room temperature for 1 h. Reaction progress was monitored by HPLC analysis. Once reaction was complete, formic acid was evaporated on rotary evaporator and residue was dissolved in water and lyophilised to obtain white powder, which was used further without any additional purifications.

The 4-SiR—COOH (20) dye TFA salt (5.9 mg, 0.01 mmol, 1 eq), DIPEA (52 μL, 0.3 mmol, 30 eq.) and HBTU (4.5 mg, 0.012 mmol, 1.2 eq.) were dissolved in 400 μL of dry MeCN and stirred at room temperature for 5 min. A solution of previously obtained deprotected DTX-C8-NH2 derivative (0.015 mmol, 1.5 eq) in MeCN and 10 μL of DIPEA were added to the reaction mixture and stirring continued for 1 hour. Reaction was monitored by HPLC analysis.

Purified by preparative HPLC (preparative column: Eurospher 100 C18, 5 μm, 250×20 mm; solvent A: acetonitrile, solvent B: H2O+0.2% v/v HCOOH; temperature 25° C., gradient A:B—5 min 40:60 isocratic, 5-30 min 40:60 to 100:0 gradient) and lyophilised from acetonitrile and water mixture. Yield 29% (3.8 mg) of slightly blue fluffy solid.

¹H NMR (400 MHz, d₆-dmso) δ 9.01 (t, J=5.5 Hz, 1H), 8.38 (d, J=9.0 Hz, 1H), 8.03-7.95 (m, 2H), 7.80-7.72 (m, 2H), 7.67 (tt, J=7.3, 1.5 Hz, 1H), 7.59 (t, J=7.7, 7.2 Hz, 2H), 7.40-7.29 (m, 4H), 7.27-7.22 (m, 1H), 7.22-7.16 (m, 1H), 7.00 (d, J=2.8 Hz, 2H), 6.70 (dd, J=8.9, 1.0 Hz, 2H), 6.67-6.60 (m, 2H), 5.96-5.87 (m, 2H), 5.41 (d, J=7.2 Hz, 1H), 5.27 (dd, J=9.0, 5.9 Hz, 1H), 5.09 (d, J=2.5 Hz, 1H), 5.01 (d, J=7.2 Hz, 1H), 4.94 (d, J=2.5 Hz, 1H), 4.90 (dd, J=9.7, 2.2 Hz, 1H), 4.58 (s, 1H), 4.41 (t, J=6.4 Hz, 1H), 4.10-3.96 (m, 3H), 3.68 (d, J=7.1 Hz, 1H), 3.30-3.25 (m, 2H), 2.92 (s, 12H), 2.34-2.25 (m, 1H), 2.24 (s, 3H), 2.17 (t, J=7.4 Hz, 2H), 2.02-1.92 (m, 1H), 1.81 (dd, J=15.3, 9.0 Hz, 1H), 1.75 (s, 3H), 1.66 (dd, J=15.4, 10.1 Hz, 1H), 1.58-1.45 (m, 7H), 1.39-1.24 (m, 6H), 1.03 (s, 3H), 0.98 (s, 3H), 0.62 (s, 3H), 0.52 (s, 3H).

¹H-¹³C NMR ((400, 101) MHz, d₆-dmso) δ (7.98 130.02), (7.76 129.42), (7.76 134.93), (7.67 133.74), (7.60 129.08), (7.36 128.57), (7.32 127.56), (7.24 125.80), (7.20 127.58), (7.00 116.66), (6.69 128.36), (6.64 114.05), (5.91 70.43), (5.42 75.21), (5.28 55.43), (5.10 74.21), (4.90 84.11), (4.42 73.99), (4.06 71.20), (4.03 75.88), (3.68 46.40), (3.30 39.76), (2.92 40.22), (2.27, 1.66 36.90), (2.24 22.83), (2.17 35.85), (1.97, 1.81 35.53), (1.75 14.10), (1.54 29.30), (1.53 10.24), (1.50 25.85), (1.34 26.88), (1.26 29.03), (1.25 29.06), (1.03 26.94), (0.98 21.26), (0.62-0.75), (0.52 0.41).

ESI-MS, positive mode: m/z=1303.6 [M+H]⁺.

HRMS (ESI) calcd for C₇₃H₈₇N₄O₁₆Si [M+H]⁺ 1303.5881, found 1303.5882.

### B.12 4-SiR-CTX (33)

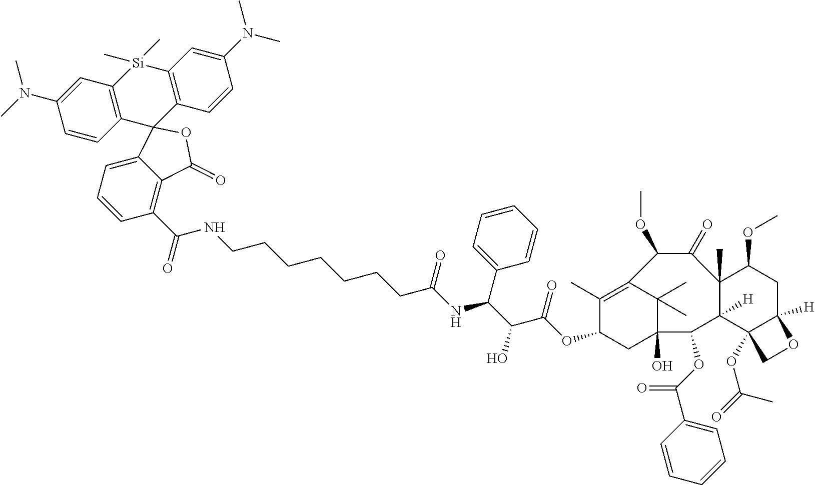

solution of CTX-C8-NHBoc (23) (14.6 mg, 0.015 mmol) in 95% formic acid (1 mL) was stirred at room temperature for 1 h. Reaction progress was monitored by HPLC analysis. Once reaction was complete, formic acid was evaporated on rotary evaporator and residue was dissolved in water and lyophilised to obtain white powder, which was used further without any additional purifications.

The 4-SiR—COOH (20) dye TFA salt (5.9 mg, 0.01 mmol, 1 eq), DIPEA (52 μL, 0.3 mmol, 30 eq.) and HBTU (4.5 mg, 0.012 mmol, 1.2 eq.) were dissolved in 400 μL of dry MeCN and stirred at room temperature for 5 min. A solution of previously obtained deprotected CTX-C8-NH2 derivative (0.015 mmol, 1.5 eq) in MeCN and 10 μL of DIPEA were added to the reaction mixture and stirring continued for 1 hour. Reaction was monitored by HPLC analysis.

Purified by preparative HPLC (preparative column: Eurospher 100 C18, 5 μm, 250×20 mm; solvent A: acetonitrile, solvent B: H2O+0.2% v/v HCOOH; temperature 25° C., gradient A:B—5 min 40:60 isocratic, 5-30 min 40:60 to 100:0 gradient) and lyophilised from acetonitrile and water mixture. Yield 51% (6.8 mg) of slightly blue fluffy solid.

¹H NMR (400 MHz, d₆-dmso) δ 8.99 (t, J=5.5 Hz, 1H), 8.34 (d, J=9.0 Hz, 1H), 7.95 (d, J=6.9 Hz, 2H), 7.74-7.71 (m, 2H), 7.66-7.61 (m, 1H), 7.56 (t, J=7.5 Hz, 2H), 7.32 (ddd, J=15.2, 8.2, 6.8 Hz, 4H), 7.21 (h, J=2.9, 1.9 Hz, 2H), 6.97 (d, J=2.8 Hz, 2H), 6.67 (dd, J=9.0, 1.2 Hz, 2H), 6.63-6.59 (m, 2H), 5.92 (dd, J=8.5, 6.4 Hz, 2H), 5.36 (d, J=7.1 Hz, 1H), 5.26 (dd, J=9.1, 5.8 Hz, 1H), 4.92 (d, J=9.5 Hz, 1H), 4.67 (s, 1H), 4.62 (s, 1H), 4.40 (dd, J=6.9, 5.8 Hz, 1H), 3.99 (s, 2H), 3.72 (dd, J=10.6, 6.5 Hz, 1H), 3.60 (d, J=7.1 Hz, 1H), 3.27 (s, 3H), 3.27-3.24 (m, 2H), 3.18 (s, 3H), 2.89 (s, 12H), 2.69-2.56 (m, 1H), 2.22 (s, 3H), 2.15 (t, J=7.4 Hz, 2H), 1.94 (dd, J=15.3, 8.9 Hz, 1H), 1.87-1.81 (m, 1H), 1.80 (s, 3H), 1.54-1.43 (m, 8H), 1.34-1.17 (m, 6H), 1.00 (s, 3H), 0.94 (s, 3H), 0.59 (s, 3H), 0.49 (s, 3H).

¹H-¹³C NMR ((400, 101) MHz, d₆-dmso) δ (7.95 129.99), (7.73 129.43), (7.73 134.94), (7.64 133.81), (7.56 129.12), (7.33 128.58), (7.30 127.56), (7.21 125.83), (7.18 127.50), (6.97 116.66), (6.66 128.36), (6.61 114.05), (5.92 70.41), (5.36 74.79), (5.26 55.36), (4.92 83.65), (4.67 82.53), (4.39 74.02), (3.99 75.71), (3.73 80.65), (3.60 46.85), (3.27 57.03), (3.27 39.77), (3.18 57.09), (2.88 40.22), (2.62, 1.47 32.13), (2.22 22.83), (2.14 35.85), (1.94, 1.85 35.32), (1.80 14.45), (1.51 29.28), (1.49 10.58), (1.47 25.86), (1.30 26.87), (1.22 29.10), (1.21 29.15), (1.00 27.13), (0.94 21.64), (0.59-0.75), (0.49 0.41).

ESI-MS, positive mode: m/z=1331.6 [M+H]⁺.

HRMS (ESI) calcd for C₇₅H₉₁N₄O₁₆Si [M+H]⁺ 1331.6194, found 1331.6184.

### B.13 4-SiR-LTX (34)

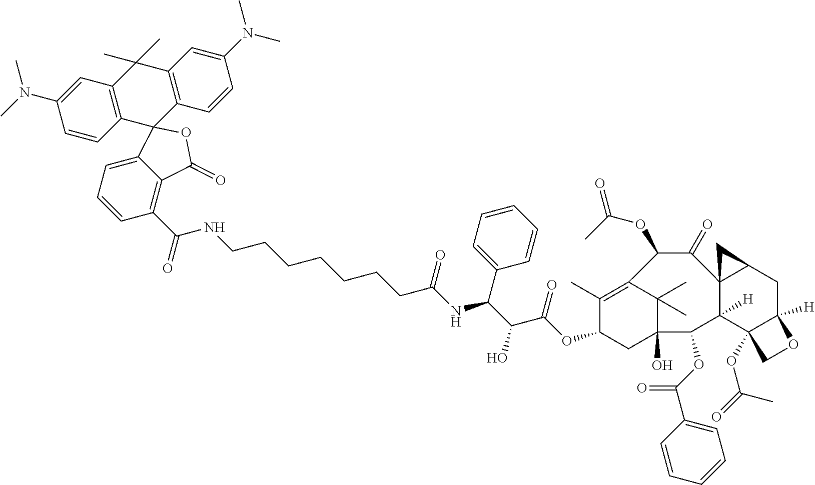

A solution of LTX-C8-NHBoc (23) (14.6 mg, stirred at room temperature for 1 h. Reaction progress was monitored by HPLC analysis. Once reaction was complete, formic acid was evaporated on rotary evaporator and residue was dissolved in water and lyophilised to obtain white powder, which was used further without any additional purifications.

The 4-SiR—COOH (20) dye TFA salt (5.7 mg, 0.01 mmol, 1 eq), DIPEA (52 μL, 0.3 mmol, 30 eq.) and HBTU (4.5 mg, 0.012 mmol, 1.2 eq.) were dissolved in 400 μL of dry MeCN and stirred at room temperature for 5 min. A solution of previously obtained deprotected LTX-C8-NH2 derivative (0.015 mmol, 1.5 eq) in MeCN and 10 μL of DIPEA were added to the reaction mixture and stirring continued for 1 hour. Reaction was monitored by HPLC analysis.

Purified by preparative HPLC (preparative column: Eurospher 100 C18, 5 μm, 250×20 mm; solvent A: acetonitrile, solvent B: H2O+0.2% v/v HCOOH; temperature 25° C., gradient A:B—5 min 40:60 isocratic, 5-30 min 40:60 to 100:0 gradient). Lyophilised from acetonitrile: water mixture.

Yield 41% (5.4 mg) of light blue fluffy solid.

¹H NMR (400 MHz, d₆-dmso) δ 8.99 (t, J=5.5 Hz, 1H), 8.35 (d, J=9.2 Hz, 1H), 8.00 (dd, J=7.0, 1.4 Hz, 2H), 7.78-7.68 (m, 2H), 7.65 (tt, J=7.3, 1.3 Hz, 1H), 7.57 (t, J=7.4 Hz, 2H), 7.37-7.25 (m, 4H), 7.22 (dd, J=5.7, 3.0 Hz, 1H), 7.14 (tt, J=7.2, 1.9 Hz, 1H), 6.97 (d, J=2.8 Hz, 2H), 6.67 (dd, J=8.8, 0.8 Hz, 2H), 6.60 (ddd, J=9.0, 2.8, 1.4 Hz, 2H), 6.09 (s, 1H), 5.99-5.84 (m, 2H), 5.41 (d, J=7.7 Hz, 1H), 5.31 (dd, J=9.2, 5.2 Hz, 1H), 4.74 (s, 1H), 4.68 (d, J=4.1 Hz, 1H), 4.46 (t, J=5.3 Hz, 1H), 3.98 (dd, J=20.6, 8.6 Hz, 2H), 3.88 (d, J=7.6 Hz, 1H), 3.25 (d, J=6.7 Hz, 2H), 2.89 (s, 12H), 2.27 (s, 4H), 2.15-1.83 (m, 9H), 1.71 (s, 3H), 1.55-1.40 (m, 5H), 1.31-1.16 (m, 7H), 1.08 (s, 3H), 1.05 (s, 3H), 0.59 (s, 3H), 0.49 (s, 3H).

¹H-¹³C NMR ((400, 101) MHz, d₆-dmso) δ (7.99 130.06), (7.74 129.45), (7.73 134.93), (7.64 133.75), (7.57 129.16), (7.30 127.71), (7.22 125.83), (7.14 127.47), (6.97 116.67), (6.66 128.37), (6.65 114.02), (6.61 114.03), (6.09 75.65), (5.92 69.96), (5.40 79.98), (5.31 55.15), (4.68 84.19), (4.46 73.97), (3.97 74.81), (3.88 37.96), (3.26 39.74), (2.88 40.21), (2.27 22.36), (2.24, 1.96 26.00), (2.12 35.84), (2.09 20.98), (2.07, 1.87 36.02), (1.99, 1.51 15.14), (1.71 14.39), (1.50 29.29), (1.44 25.87), (1.28 26.86), (1.22 26.99), (1.20 29.11), (1.14 31.67), (1.08 21.82), (1.05 26.34), (0.59-0.76), (0.49 0.41).

ESI-MS, positive mode: m/z=1327.6 [M+H]⁺.

HRMS (ESI) calcd for C₇₅H₈₇N₄O₁₆Si [M+H]⁺ 1327.5881, found 1327.5895.

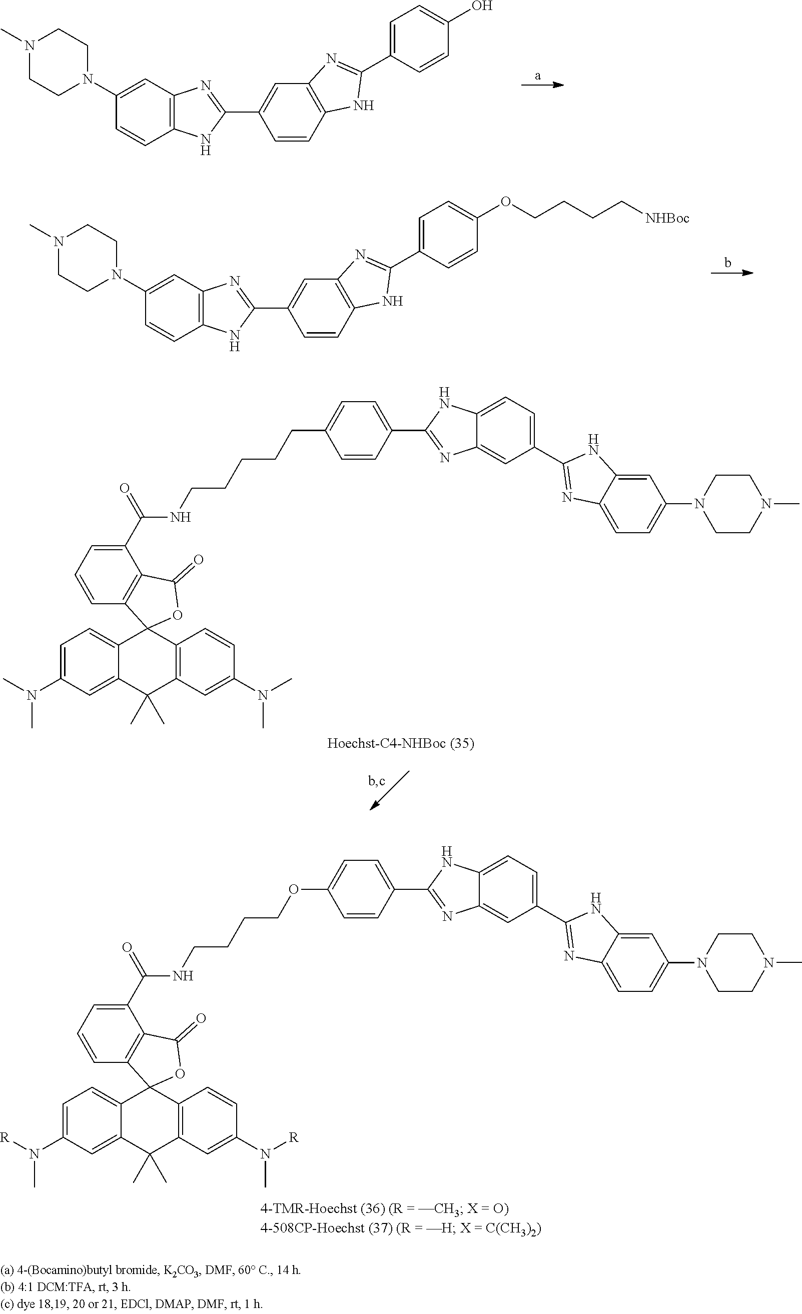

### B.14 Hoechst-C4-NHBoc (35)

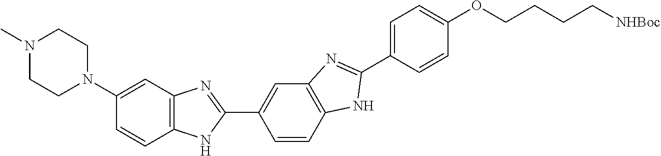 [SMILES: CN1CCN(c2ccc3[nH]c(-c4ccc5[nH]c(-c6ccc(OCCCCNC(=O)OC(C)(C)C)cc6)nc5c4)nc3c2)CC1]

The free base of Hoechst 33258 was prepared by dissolving commercial Hoechst 33258 trihydrochloride (500 mg, 0.936 mmol) in H₂O (30 mL) and adding a solution of K₂CO₃ (388 mg, 2.81 mmol, 3 eq.) in H₂O (10 mL). The precipitate thus formed was isolated by filtration, washed with H₂O and lyophilised from water 1,4-dioxane mixture. The resulting Hoechst 33258 base (397 mg, 0.936 mmol, 1 eq.) was suspended in dry DMF (1 mL). K₂CO₃ (388 mg, 2.81 mmol, 3 eq.) was added followed by 4-(Boc-amino)butyl bromide (283 mg, 1.12 mmol, 1.2 eq.). The reaction was heated at 60° C. for 14 h. The reaction mixture was then cooled to room temperature, DMF was evaporated under reduced pressure. The residue was suspended in DCM and deposited on celite by evaporating the solvent. Product was isolated by flash column chromatography (Büchi Reveleris HP silica 40 g; gradient 20% to 90% CH₂Cl₂—CH₂Cl₂:MeOH:NH_{3(aq)} [9:1:0.2]) as yellow solid, yield 306 mg (55%).

¹H NMR (600 MHz, CD₃OD) δ 8.22 (d, J=1.6 Hz, 1H), 8.01 (d, J=8.8 Hz, 2H), 7.92 (dd, J=8.4, 1.6 Hz, 1H), 7.66 (d, J=8.4 Hz, 1H), 7.50 (d, J=8.7 Hz, 1H), 7.13 (d, J=2.3 Hz, 1H), 7.01-7.07 (m, 3H), 4.03 (t, J=6.4 Hz, 2H), 3.23-3.33 (m, 4H), 3.11 (t, J=7.0 Hz, 2H), 2.79-2.84 (m, 4H), 2.48 (s, 3H), 1.80 (p, J=7.2 Hz, 2H), 1.65 (p, J=7.2 Hz, 2H), 5 1.43 (s, 9H).

¹³C NMR (126 MHz, CD₃OD) b 169.6, 162.4, 158.4, 155.2, 153.7, 149.2, 148.3, 140.4, 136.0, 129.5, 125.5, 122.8, 122.4, 116.5, 116.3, 116.0, 113.8, 102.5, 79.9, 68.9, 56.0, 51.4, 45.6, 41.1, 28.8, 27.7.

HRMS (ESI) calcd for C₃₄H₄₂N₇O₃ [M+H]⁺ 596.3344, found 596.3340.

### B.15 4-TMR-Hoechst (36)

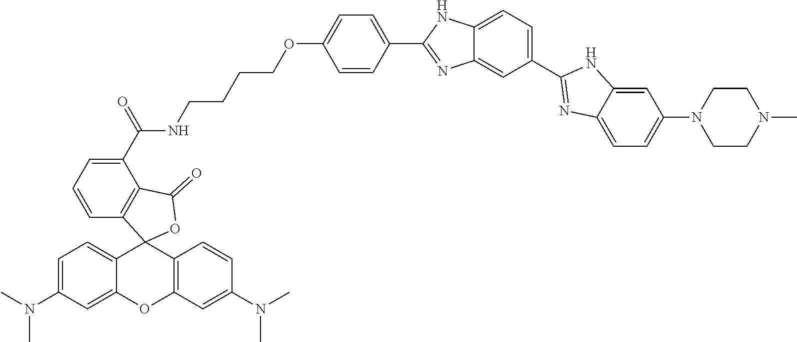

Trifluoroacetic acid (0.2 mL) was added dropwise to a solution of Hoechst-C4-NHBoc (35) (16.5 mg, 0.0276 mmol) in CH₂Cl₂ (0.8 mL). The resulting intense yellow solution was stirred at room temperature for 3 h. The reaction mixture was then evaporated to dryness, the residue was re-evaporated three times with MeOH to remove excess trifluoroacetic acid. The residue was lyophilized from aqueous dioxane. Deprotected product obtained quantatively as trifluoroacetic acid salt ([Hoechst-(CH₂)₄NH₃]⁴⁺[CF₃COO⁻]₄) as yellow solid. Compound used in further step without additional purification.

4-TMR-COOH-TFA salt (10 mg, 0.0184 mmol, 1 eq.), EDCl-HCl (8.4 mg, 0.0276 mmol, 1.5 eq.) DMAP (0.11 mmol, 13.4 mg, 6 eq.) and previously obtained [Hoechst-(CH₂)₄NH₃]⁴⁺[CF₃COO⁻]₄ (26 mg, 0.0276 mmol, 1.5 eq.) were dissolved in 1 mL of dry DMF and stirred for 1 hour. Then DMF was removed at rt under reduced pressure. The product was purified by preparative HPLC (preparative column: Eurospher 100 C18, 5 μm, 250×20 mm; solvent A: acetonitrile, solvent B: H2O+0.2% v/v HCOOH; temperature 25° C., gradient A:B—5 min 20:80 isocratic, 5-30 min 20:80 to 70:30 gradient). Water acetonitrile mixture was removed by rotary evaporator and product was purified one more time by flash chromatography (silica-gel cartridge: Interchim Puriflash 12 g, 15 μm column, gradient 20% to 80% CH₂Cl₂—CH₂Cl₂:MeOH:NH₃(aq) [85:15:2]). Solvents were removed and a product was lyophilised from water acetonitrile mixture to obtain 9 mg (54%) of purple solid.

¹H NMR (400 MHz, CD₃OD+CF₃COOD) δ 8.58 (dd, J=1.7, 0.7 Hz, 1H), 8.25 (dd, J=8.7, 1.7 Hz, 1H), 8.18 (d, J=9.0 Hz, 2H), 8.07 (dd, J=8.7, 0.7 Hz, 1H), 7.83-7.79 (m, 2H), 7.76 (d, J=9.1 Hz, 1H), 7.51 (dd, J=6.2, 2.7 Hz, 1H), 7.44 (dd, J=9.2, 2.2 Hz, 1H), 7.36 (d, J=2.2 Hz, 1H), 7.29 (d, J=9.0 Hz, 2H), 7.23 (d, J=9.5 Hz, 2H), 7.07 (dd, J=9.5, 2.5 Hz, 2H), 6.97 (d, J=2.4 Hz, 2H), 4.22 (t, J=6.2 Hz, 2H), 3.97 (d, J=13.2 Hz, 2H), 3.68 (d, J=12.1 Hz, 2H), 3.49 (t, J=6.9 Hz, 2H), 3.36 (d, J=11.3 Hz, 2H), 3.30 (s, 12H), 3.19 (d, J=11.9 Hz, 2H), 3.00 (s, 3H), 2.03-1.94 (m, 2H), 1.87 (dd, J=8.7, 6.0 Hz, 2H).

¹³C NMR (101 MHz, CD₃OD+CF₃COOD) δ 171.1, 169.2, 165.6, 159.1, 158.8, 154.3, 151.1, 149.5, 139.2, 134.6, 133.9, 133.7, 132.5, 131.9, 131.5, 130.2, 128.0, 126.3, 121.7, 120.9, 120.0, 118.1, 117.5, 117.2, 116.4, 115.7, 115.5, 115.2, 115.0, 114.6, 112.4, 101.0, 97.5, 69.5, 54.6, 48.4 (visible in HSQC), 43.6, 41.0, 40.7, 27.5, 26.9.

ESI-MS, positive mode: m/z=908.5 [M+H]⁺.

HRMS (ESI) calcd for C₅₄H₅₄N₉O₅ [M+H]⁺ 908.4242, found 908.4230.

### B.16 4-580CP-Hoechst (37)

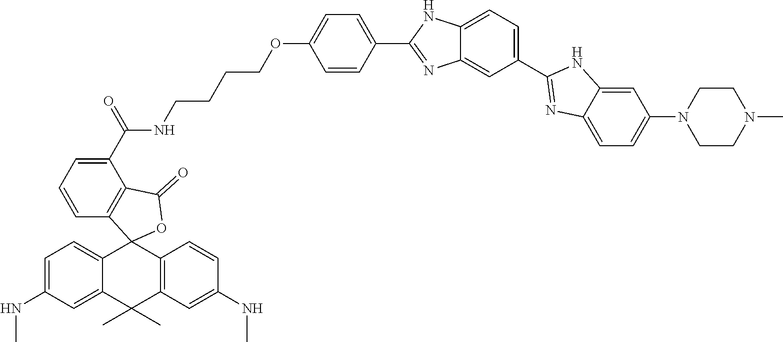 [SMILES: CNc1ccc2c(c1)C(C)(C)c1cc(NC)ccc1C21OC(=O)c2c(C(=O)NCCCCOc3ccc(-c4nc5cc(-c6nc7ccc(N8CCN(C)CC8)cc7[nH]6)ccc5[nH]4)cc3)cccc21]

Trifluoroacetic acid (0.2 mL) was added dropwise to a solution of Hoechst-C4-NHBoc (35) (16.5 mg, 0.0276 mmol) in CH₂Cl₂ (0.8 mL). The resulting intense yellow solution was stirred at room temperature for 3 h. The reaction mixture was then evaporated to dryness, the residue was re-evaporated three times with MeOH to remove excess trifluoroacetic acid. The residue was lyophilized from aqueous dioxane. Deprotected product obtained quantatively as trifluoroacetic acid salt ([Hoechst-(CH₂)₄NH₃]⁴⁺[CF₃COO⁻]₄) as yellow solid. Compound used in further step without additional purification.

4-580CP-COOH-TFA salt (0.0184 mmol, 10 mg, 1 eq.), EDCl-HCl (0.0276 mmol, 8.4 mg, 1.5 eq.) DMAP (0.11 mmol, 13.4 mg, 6 eq.) and [Hoechst-(CH₂)₄NH₃]⁴⁺[CF₃COO⁻]₄ (0.0276, 26 mg, 1.5 eq.) were dissolved in 1 mL of dry DMF and stirred for 1 hour. Then DMF was removed at rt under reduced pressure. The product was purified by preparative HPLC (preparative column: Eurospher 100 C18, 5 μm, 250×20 mm; solvent A: acetonitrile, solvent B: H₂O+0.2% v/v HCOOH; temperature 25° C., gradient A:B—5 min 20:80 isocratic, 5-30 min 20:80 to 70:30 gradient). Water acetonitrile mixture was removed by rotary evaporator and product was purified one more time by flash chromatography (silica-gel cartridge: Interchim Puriflash 12 g, 15 μm column, gradient 20% to 80% CH₂Cl₂—CH₂Cl₂:MeOH:NH_{3(aq)} [85:15:2]). Solvents were removed and a product was lyophilised from water acetonitrile mixture to obtain 6 mg (36%) of dark-violet solid.

¹H NMR (400 MHz, CD₃OD+CF₃COOD) δ 8.39 (s, 1H), 8.17-8.04 (m, 3H), 7.95 (d, J=8.6 Hz, 1H), 7.86-7.78 (m, 2H), 7.76 (d, J=8.9 Hz, 1H), 7.48-7.39 (m, 2H), 7.36 (s, 1H), 7.21-7.12 (m, 4H), 7.09 (d, J=9.1 Hz, 2H), 6.68 (dd, J=9.1, 2.3 Hz, 2H), 4.13 (t, J=5.7 Hz, 2H), 3.98 (d, J=13.1 Hz, 2H), 3.77-3.71 (m, 2H), 3.52 (t, J=6.0 Hz, 2H), 3.35 (d, J=10.6 Hz, 2H), 3.27 (d, J=13.3 Hz, 2H), 3.07 (s, 6H), 3.03 (s, 3H), 1.99-1.85 (m, 4H), 1.83 (s, 3H), 1.70 (s, 3H).

¹³C NMR (101 MHz, CD₃OD+CF₃COOD) b 169.7, 168.6, 162.7, 161.7, 161.4, 157.3, 156.7, 153.7, 149.2, 148.7, 137.4, 136.8, 136.7, 133.2, 132.0, 131.1, 130.3, 129.2, 128.1, 126.9, 124.7, 123.2, 120.7, 118.6, 118.1, 115.2, 115.2, 114.3, 113.7, 112.7, 111.0, 99.7, 67.8, 53.2, 47.1, 42.3, 41.1, 39.4, 34.2, 30.8, 28.9, 26.2, 25.5.

ESI-MS, positive mode: m/z=906.4 [M+H]⁺.

HRMS (ESI) calcd for C₅₅H₅₆N₉O₄ [M+H]⁺ 906.4450, found 906.4455.

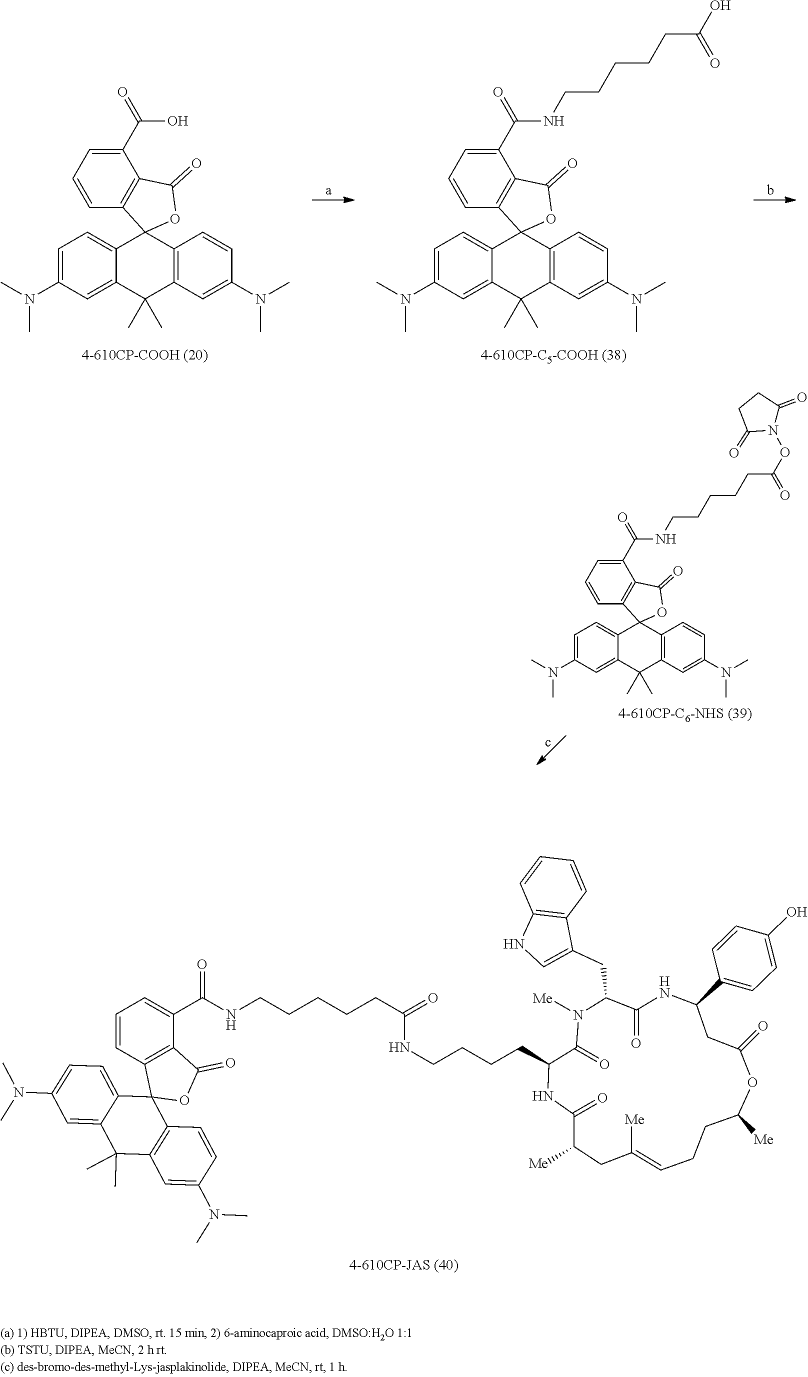

### B.17 4-610CP-C₅—COOH (38)

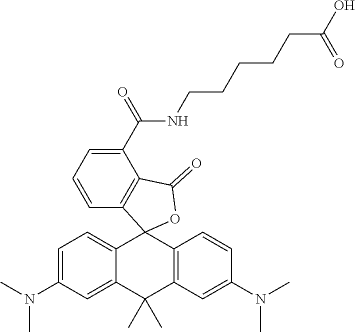 [SMILES: CN(C)c1ccc2c(c1)C(C)(C)c1cc(N(C)C)ccc1C21OC(=O)c2c(C(=O)NCCCCCC(=O)O)cccc21]

4-610CP-COOH (20) TFA salt (0.0175 mmol, 10 mg, 1 eq.), DIPEA (15 μL, 0.0875 mmol, 5 eq.) and HBTU (0.021 mmol, 8.0 mg, 1.2 eq.) were dissolved in 400 μL of dry DMSO and stirred at room temperature for 15 min. A solution of 6-aminocaproic acid (0.0175 mmol, 2.3 mg, 1 eq.) in 200 μL of DMSO:H2O mixture (1:1) was added to the reaction mixture and stirring continued for 1 hour. Reaction mixture was neutralised with formic acid and product was purified by preparative HPLC (preparative column: Eurospher 100 C18, 5 μm, 250×20 mm; solvent A: acetonitrile, solvent B: H2O+0.2% v/v HCOOH; temperature 25° C., gradient A:B—5 min 40:60 isocratic, 5-30 min 40:60 to 80:20 gradient). Product lyophilised from acetonitrile: water mixture to obtain 7 mg (70%) of blue powder.

¹H NMR (400 MHz, CD₃CN) δ 9.74 (t, J=5.1 Hz, 1H), 8.28 (dd, J=7.7, 1.0 Hz, 1H), 7.72 (t, J=7.7 Hz, 1H), 7.08 (dd, J=7.7, 1.0 Hz, 1H), 6.96 (d, J=2.3 Hz, 2H), 6.64-6.53 (m, 4H), 3.49 (q, J=7.0 Hz, 2H), 2.97 (s, 13H), 2.31 (t, J=7.4 Hz, 2H), 2.20 (s, 1H), 1.86 (s, 3H), 1.75 (s, 3H), 1.73-1.61 (m, 4H), 1.56-1.44 (m, 2H).

¹³C NMR (101 MHz, CD₃CN) b 175.08, 172.55, 164.64, 158.41, 152.15, 147.76, 136.10, 134.66, 132.21, 129.55, 127.07, 123.70, 119.05, 112.78, 110.31, 40.67, 40.59, 39.27, 35.31, 34.14, 33.58, 29.72, 27.25, 25.32.

ESI-MS, positive mode: m/z=570.3 [M+H]⁺.

HRMS (ESI) calcd for C₃₄H₄₀N₃O₅[M+H]⁺ 570.2962, found 570.2965.

### B.18 4-610CP-C₆—NHS (39)

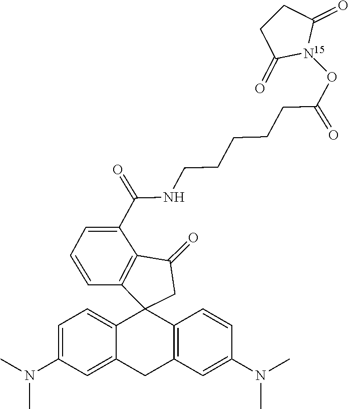

4-610CP-C₅—COOH (37) (0.021 mmol, 12.0 mg), TSTU (0.0274 mmol, 8.1 mg) and DIPEA (36 μL, 0.2064 mmol) were dissolved in 500 μL of MeCN and stirred for 1 hour. Then MeCN was removed by rotary evaporator and obtained product was purified by flash chromatography (silica-gel cartridge: Interchim Puriflash 12 g, 15 μm column, gradient 20% to 100% DCM-EtOAc). The solvents were removed and the obtained NHS ester was redissolved in 1,4-dioxane, microfiltered through a 0.45 μm PTFE membrane filter and lyophilized to obtain 10 mg (71%) of blue powder.

¹H NMR (400 MHz, Acetonitrile-d₃) δ 9.71 (s, 1H), 8.28 (dd, J=7.7, 1.0 Hz, 1H), 7.74 (t, J=7.7 Hz, 1H), 7.09 (dd, J=7.7, 1.0 Hz, 1H), 6.97 (d, J=2.3 Hz, 2H), 6.71-6.54 (m, 4H), 3.51 (td, J=6.9, 5.4 Hz, 2H), 2.99 (s, 12H), 2.75 (s, 4H), 2.67 (t, J=7.4 Hz, 2H), 1.87 (s, 3H), 1.85-1.77 (m, 2H), 1.76 (s, 3H), 1.76-1.68 (m, 2H), 1.63-1.54 (m, 2H).

ESI-MS, positive mode: m/z=667.2 [M+H]⁺.

### B.19 4-610CP-JAS (40)

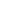

The 4-610CP-C₆—NHS (39) (2.7 mg, 0.0041 mmol) ester was dissolved in 400 μL of MeCN followed by the addition of deprotected des-bromo-des-methyl-Lys-jasplakinolide (0.00342 mmol, 2.3 mg) (Tannert, R., Milroy, L. G., Ellinger, B., Hu, T. S., Arndt, H. D., Waldmann, H., J Am Chem Soc, 2010, 132 (9), 3063-3077) and DIPEA (12 μL, 0.0688 mmol). The reaction mixture was stirred for 1 hour and purified by preparative HPLC (preparative column: Eurospher 100 C18, 5 μm, 250×20 mm; solvent A: acetonitrile, solvent B: H2O+0.2% v/v HCOOH; temperature 25° C., gradient A:B—5 min 30:70 isocratic, 5-30 min 30:70 to 100:0 gradient). Product lyophilised from acetonitrile: water mixture to obtain 1.8 mg (48%) of light blue powder.

¹H NMR (400 MHz, d₆-dmso) δ 10.79 (d, J=1.8 Hz, 1H), 9.27 (s, 1H), 9.11 (t, J=5.5 Hz, 1H), 8.61 (d, J=8.8 Hz, 1H), 7.75 (d, J=7.6 Hz, 1H), 7.70-7.60 (m, 4H), 7.26 (d, J=8.1 Hz, 1H), 7.10 (d, J=8.6 Hz, 2H), 7.03 (d, J=2.3 Hz, 1H), 7.02-6.97 (m, 2H), 6.92 (t, J=7.2 Hz, 1H), 6.88 (d, J=2.1 Hz, 2H), 6.67 (d, J=8.5 Hz, 2H), 6.58-6.50 (m, 4H), 5.49 (dd, J=11.3, 5.1 Hz, 1H), 5.22-5.11 (m, 1H), 4.89 (t, J=7.1 Hz, 1H), 4.64 (h, J=6.4 Hz, 1H), 4.58-4.44 (m, 1H), 3.35-3.31 (m, 2H), 3.08-2.96 (m, 4H), 2.91 (s, 12H), 2.89-2.71 (m, 3H), 2.65 (dd, J=14.7, 11.3 Hz, 1H), 2.55 (dd, J=14.7, 3.2 Hz, 1H), 2.53-2.48 (m, 2H), 2.14 (dd, J=14.6, 11.5 Hz, 1H), 2.05 (t, J=7.5 Hz, 2H), 1.86-1.75 (m, 5H), 1.74-1.65 (m, 4H), 1.61-1.49 (m, 4H), 1.45 (s, 3H), 1.38-1.33 (m, 2H), 1.22-1.20 (m, 2H), 1.13 (d, J=6.3 Hz, 3H), 1.10-1.00 (m, 2H), 0.90 (d, J=6.8 Hz, 3H), 0.85-0.71 (m, 4H).

ESI-MS, positive mode: m/z=1247.6 [M+Na]⁺.

HRMS (ESI) calcd for C₇₂H₈₉N₈O₁₀ [M+H]⁺ 1225.6696, found 1225.6689.

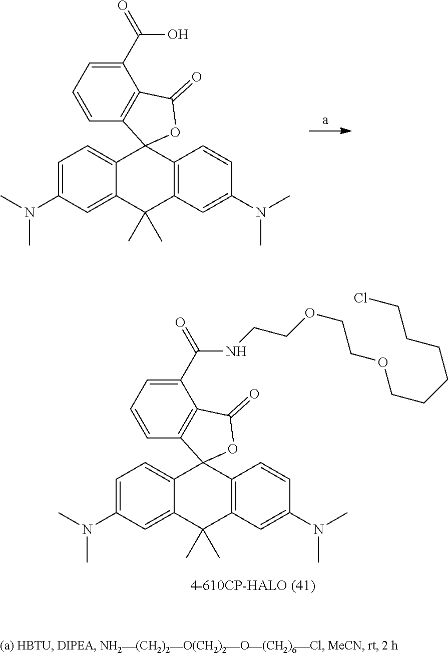

### B.20 4-610CP-HALO (40)

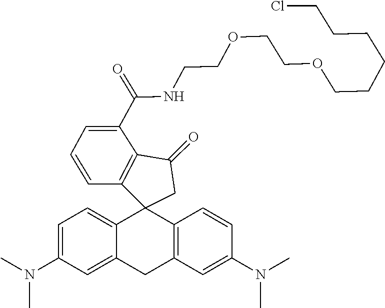 [SMILES: CN(C)c1ccc2c(c1)Cc1cc(N(C)C)ccc1C21CC(=O)c2c(C(=O)NCCOCCOCCCCCCCl)cccc21]

4-610CP-COOH (20)—TFA salt (10 mg, 0.0175 mmol, 1 eq.), DIPEA (15 μL, 0.0875 mmol, 5 eq.) and HBTU (8.0 mg, 0.021 mmol, 1.2 eq.) were dissolved in 400 μL of dry MeCN and stirred at room temperature for 5 min.

A solution of NH₂—(CH₂)₂—O—(CH₂)₂—O—(CH₂)₆—Cl (6 mg, 0.0267 mmol, 1.53 eq.) in 100 μL of dry MeCN was added to the reaction mixture and stirring continued for 1 hour. Reaction mixture was neutralised with formic acid and product was purified by preparative HPLC (preparative column: Eurospher 100 C18, 5 μm, 250×20 mm; solvent A: acetonitrile, solvent B: H2O+0.2% v/v HCOOH; temperature 25° C., gradient A:B—5 min 40:60 isocratic, 5-30 min 30:70 to 100:0 gradient). Product was purified one more time by flash chromatography (silica-gel cartridge: Interchim Puriflash 12 g, 15 μm column, gradient 20% to 80% CH₂Cl₂—CH₂Cl₂:MeOH [90:10]). Solvents were removed and a product was lyophilised from acetonitrile: water mixture to obtain 7 mg (60%) of blue-violet powder.

¹H NMR (400 MHz, d₆-DMSO) δ 9.34 (t, J=5.5 Hz, 1H), 7.83 (dd, J=7.5, 1.0 Hz, 1H), 7.70 (t, J=7.6 Hz, 1H), 7.06 (dd, J=7.7, 1.0 Hz, 1H), 6.91 (d, J=2.2 Hz, 2H), 6.63-6.52 (m, 4H), 3.63 (t, J=5.9 Hz, 2H), 3.60-3.49 (m, 8H), 3.38 (t, J=6.5 Hz, 2H), 2.94 (s, 12H), 1.82 (s, 3H), 1.72 (s, 3H), 1.71-1.61 (m, 2H), 1.47 (q, J=6.9 Hz, 2H), 1.37-1.26 (m, 4H).

¹³C NMR (126 MHz, d₆-DMSO) b 169.4, 164.7, 156.5, 150.6, 146.0, 135.0, 134.5, 129.3, 128.4, 125.2, 121.9, 118.1, 111.7, 109.1, 86.6, 70.2, 69.7, 69.5, 68.9, 45.4, 39.9, 39.8, 37.9, 34.6, 33.5, 32.0, 29.1, 26.1, 24.9.

ESI-MS, positive mode: m/z=662.3 [M+H]⁺.

### Example 1

### Characterization of Rhodamine 4′-Isomer Dyes and 4′-Isomer Based Fluorescent Probes

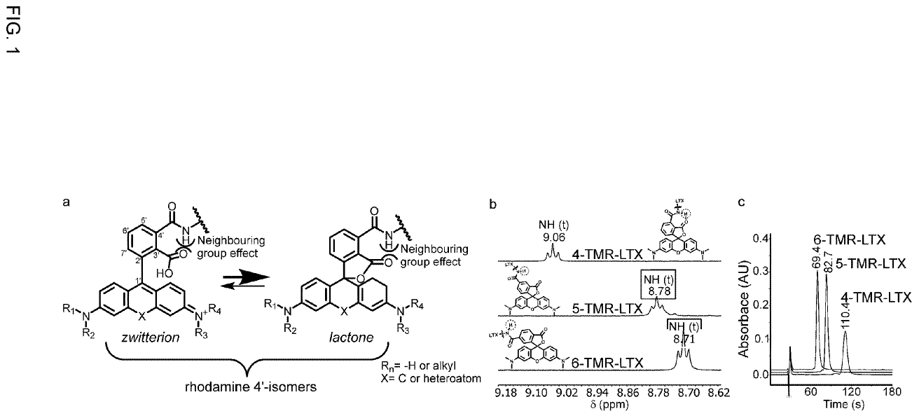

The neighboring group effect (NGE) as described above could be detected by NMR or HPLC methods (FIG. 1): Rhodamine 4′-isomers containing NGE show significant NMR chemical shift of the interacting atoms which are not bound via covalent chemical bond and increased HPLC retention times. The NGE is most profound after attachment of the linker or a ligand by conversion of the carboxylic group to carboxamide.

FIG. 1 shows the occurrence of neighboring group effect in the 4′-isomers of rhodamines. a, Neighboring group effect shifts spirolactone-zwitterion equilibrium of rhodamines. X═O, S, NR₅, CR₅R₆, SO₂, P(O)OH, P(O)OR₅, SiR₅R₆, GeR₅R₆, Rₙ=alkyl. b, Chemical shift differences of amide proton of TMR-LTX regioisomeric probes. c, Comparison of retention times of TMR-LTX regioisomeric probes in HPLC analysis with SB-C18 column and isocratic elution conditions (75:25 MeOH:H₂O 25 mM HCOONH₄ pH=3.6).

The ability of the fluorophores to switch between spirolactone and zwitterion states can be characterized by measuring the absorbance in water-1,4-dioxane mixtures with known dielectric constant (Åkerlöf G, Short A O. J Am Chem Soc 1936, 58(7): 1241-1243). D₅₀ is commonly used as numeric value for the evaluation of the equilibrium changes and represents a dielectric constant at which absorbance is halved.

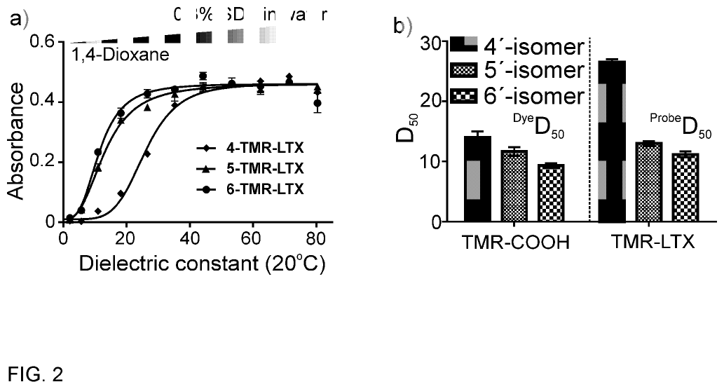

FIG. 2 a) shows graphs representing the absorbance of TMR-LTX positional isomers at λₘₐₓ versus dielectric constant (D) of 1,4-dioxane-water mixtures and FIG. 2 b) shows ^{Dye}D₅₀ values of positional isomers of TMR-COOH and ^{probe}D₅₀ of TMR-LTX.

As evident from FIG. 2, for 5′- and 6′-isomers, the ^{Probe}D₅₀ values of conjugates are only marginally higher than ^{Dye}D₅₀ of corresponding fluorophores. In contrast to this, ^{Probe}D₅₀ values of 4′-isomers conjugates are increased compared to the corresponding ^{Dye}D₅₀ values of the fluorophore, and significantly higher than conjugates ^{Probe}D₅₀ values of 5′- and 6′-isomers. In addition, NMR shift value of —CONH— proton of the probe is significantly increased (>0.3 ppm) in 4′-isomers compared to the corresponding values of 5′- and 6′-isomers. This indicates that the conversion of carboxyl group to amide modulates NGE further and induces higher spirolactone content at equilibrium and thus implies higher hydrophobicity of 4′-isomer conjugates. Most importantly, it demonstrates the major difference between 5′/6′-isomers and 4′-isomer of rhodamine class dyes.

The following Table 1 shows comparative D₅₀ values for different positional isomers of selected rhodamine probes.

| | 1,4-Dioxane-Water with constant 0.3% SDS additive | | | |
|---|---|---|---|---|
| | TMR-LTX | 580CP-LTX | 610CP-CTX | SiR-CTX |
| Probe | D₅₀ | D₅₀ | D₅₀ | D₅₀ |
| 4′-isomer | 26.5 ± 0.5 | 62 ± 1 | 65 ± 2 | >80 |
| 5′-isomer | 13.0 ± 0.4 | 40.0 ± 0.6 | 39.7 ± 0.2 | 66.8 ± 0.3 |
| 6′-isomer | 11.2 ± 0.5 | 36.0 ± 0.7 | 36.4 ± 0.4 | 66.5 ± 0.4 |

The following Table 2 shows comparative chemical shift of amide NH proton in DMSO-d6 of isomeric dye-C8-taxane conjugates

| | Conjugate isomer | | |
|---|---|---|---|
| Probe | 4′-isomer | 5′-isomer | 6′-isomer |
| TMR-LTX | 9.07 | 8.76 | 8.62 |
| 580CP-LTX | 9.14 | 8.72 | 8.69 |
| 610CP-CTX | 9.09 | 8.73 | 8.66 |
| SiR-CTX | 8.99 | 8.75 | 8.69 |
| Average | 9.07 ± 0.06 | 8.74 ± 0.01 | 8.67 ± 0.03 |

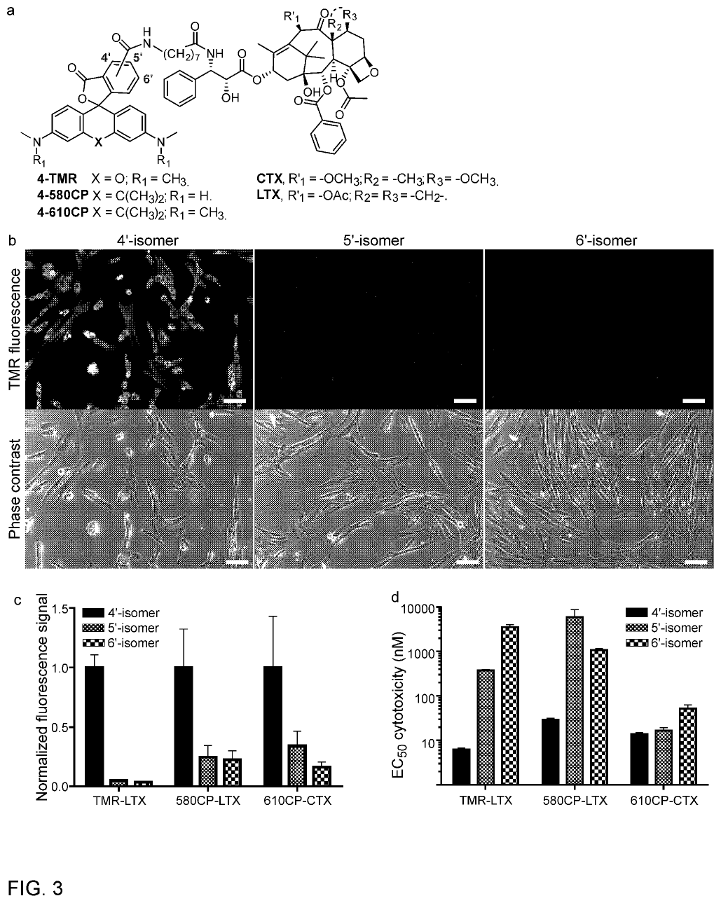

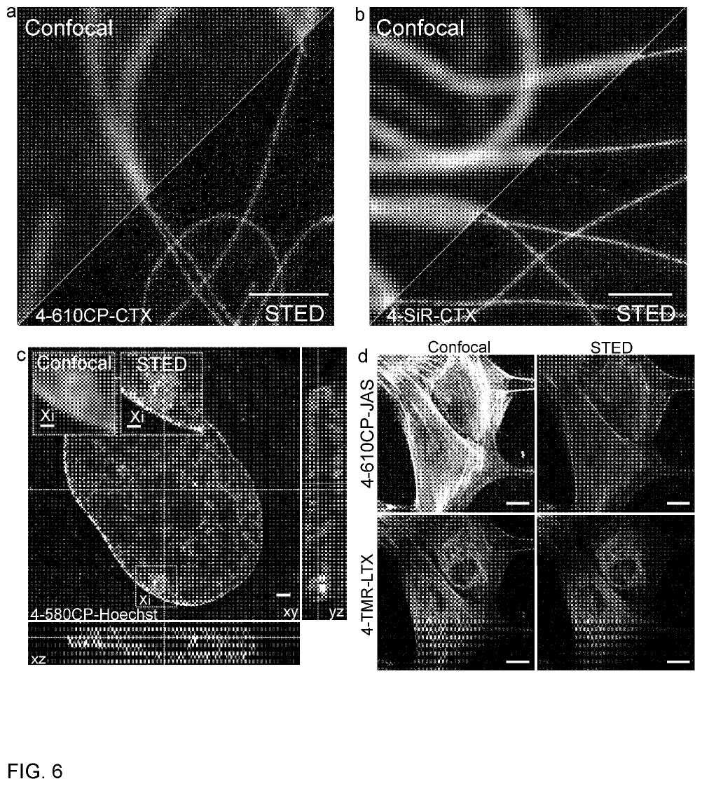

As demonstrated in further experiments and FIG. 3, this neighboring group effect results in dramatically increased cell membrane permeability while keeping all photophysical properties almost unchanged. As an example, the inventors prepared the larotaxel derivative (4-TMR-LTX) for labelling of tubulin in living cells (FIG. 3a). 4-TMR-LTX demonstrates excellent membrane permeability compared to widely used 5′- and 6′-isomers and permits the highly specific tubulin labelling in mammalian cells by simply incubating cells with 4-TMR-LTX (FIG. 3b,c). To prove the increased membrane permeability, the inventors relied on the property of the targeting moiety, larotaxel, to perturb cell cycle. 4-TMR-LTX induce accumulation of subG1 phase cells almost as efficiently as parent compound. In contrast, the 5′- and 6′-isomers showed much higher EC₅₀ (FIG. 6d). This demonstrates the superior permeability, labeling specificity and biocompatibility of the 4′-isomer. Importantly, the use of the observed NGE enhanced cell membrane permeability could be extended to other rhodamine like fluorophores-carbopyronines (580CP and 610CP) 4′-isomers derivatives (FIG. 3b-d).

As mentioned above, FIG. 3. illustrates the tubulin labelling of mammalian cells: a, structures of the tubulin probes. b, Wide-field fluorescence microscopy of living primary fibroblasts stained 100 nM TMR-LTX isomers for 1 h at 37° C. Cells were washed once with HBSS and imaged in growth DMEM media. Inserts shows zoomed-in images. Scale bars: large field of view—100 μm, insert −10 μm. Hoechst staining is shown in cyan and all tubulin probes were shown in magenta. c, Quantification of fluorescence signal in the cytoplasm of living cells stained with tubulin probes. Data were presented as mean±s.e.m., N=3 independent experiments, each time n>100 cells were quantified. d, Cytotoxicity of tubulin fluorescent probes presented as half maximal effective concentration (EC50) after 24 h incubation at 37° C. in growth media. Cytotoxicity was determined as fraction of cells population containing less then single set of genetic material (sub G1 DNA content). Data were presented as mean±s.e.m., N=3 independent experiments, each time n>100 cells were quantified.

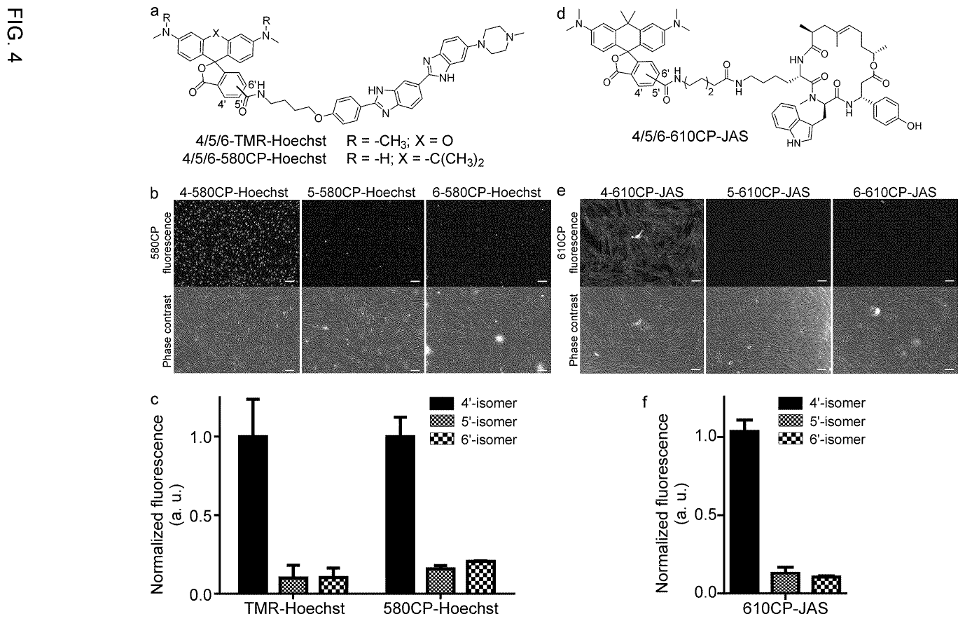

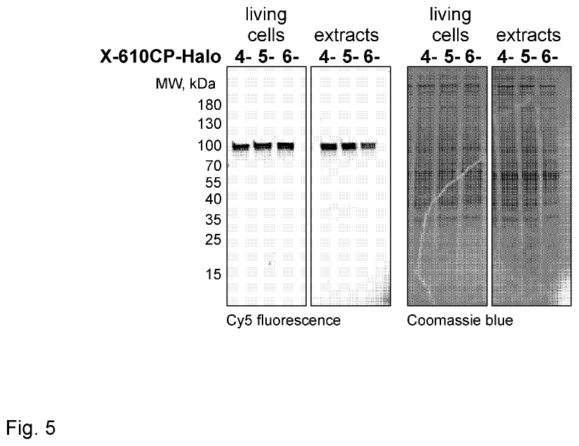

Furthermore, said increased cell permeability and favourable optical properties are also observed with multiple targeting moieties, as demonstrated with ligands targeting actin, DNA and Halo-tag (FIGS. 4 and 5). This demonstrates that rhodamine 4′-isomers represent a unique platform for the development of biocompatible fluorescent probes, in which the rhodamine core structure is derivatized with: (i) ligands that specifically bind to other biomolecules in vitro or in vivo, (ii) reactive groups such as activated esters or (iii) molecules that can control the fluorescence properties of the fluorescent dyes, like quenchers.

FIG. 4 shows the performance of DNA and actin fluorescent probes based on rhodamines' positional isomers: a, Structure of DNA probes showing attachment point positional isomerism. b, Wide-field microscopy images of living primary fibroblasts were stained with 100 nM 4/5/6-580CP-Hoechst (magenta) for 1 h at 37° C. overlaid with of the light transmission (grey). Cells washed once with HBSS and imaged in growth DMEM media. Inserts show zoomed-in images. Scale bars: large field of view 100 μm, inserts−10 μm. c, Quantification of DNA probes' fluorescence signal in the nuclei. Data presented as mean s.d., N=3 independent experiments, each n>100 cells. d, Structure of actin probes attachment point positional isomerism. e, Wide-field microscopy overlay images of the light transmission (grey) and fluorescence channels: Hoechst—blue, 610CP—yellow. Living primary fibroblasts stained with 100 nM 4/5/6-610CP-JAS for 1 h at 37° C. Cells were washed once with HBSS and imaged in growth DMEM media. Inserts show zoomed-in images. Scale bars: large field of view 100 μm, inserts−10 μm. f, Quantification of 4/5/6-610CP-JAS fluorescence signal in the cytoplasm of living cells. Data were presented as mean s.d., N=3 independent experiments, each n>100 cells.

FIG. 5 shows the SDS-PAGE in-gel fluorescence of 100 nM Halo-tag purified protein labeled with 100 nM 4-610CP-Halo substrate for the indicated periods of time at 37° C.

### Example 2

### STED Optical Microscopy of Living Cells

The novel fluorescent probes were applied for STED nanoscopy imaging in living cells. For these experiments, STED optical microscopy was performed as indicated above.

The inventors obtained an unprecedented quality of images at resolution below diffraction limit. A combination of TMR and 610CP was successfully applied for two-color imaging of living cells.

FIG. 6 shows STED nanoscopy imaging of living cells stained with rhodamine 4′-isomer probes: a, Confocal and STED microscopy images of microtubules in living human fibroblasts stained with 100 nM 4-610CP-CTX for 1 h at 37° C. Scale bar 1 μm. b, Confocal and STED microscopy images of microtubules in living human fibroblasts stained with 100 nM 4-SiR-CTX for 1 h at 37° C. Scale bar 1 μm. c, Deconvolved STED image of human female primary fibroblast nucleus stained with 100 nM 4-580CP-Hoechst showing the inactivated X chromosome (Xi). Insets—confocal and STED microscopy zoomed-in images of Xi region. Scale bars: 500 nm—insets, 1 μm—main image. d, Two-colour STED no-wash image of primary human fibroblasts stained with 100 nM 4-610CP-JAS and 10 nM 4-TMR-LTX for 1 h at 37° C. Scale bar 10 μm.

1. A fluorescent dye which is a rhodamine 4′-isomer having the following general structural formula A: 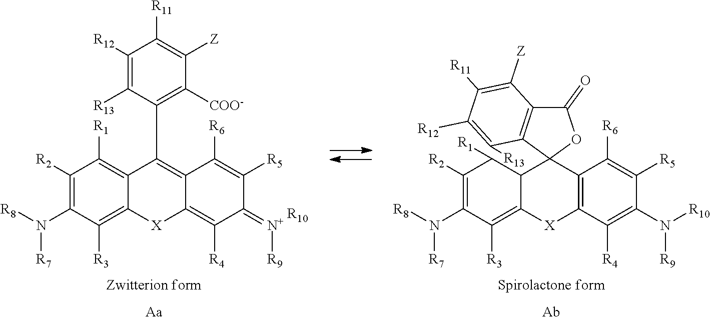
wherein
R₁, R₂, R₃, R₄, R₅, R₆, R₇, R₈, R₉, R₁₀, R₁₁, R₁₂ and R₁₃ are independently selected from H, halogen, D, CN, OH, O(alkyl), O(aryl), SH, S(alkyl), S(aryl), S(O)(alkyl), S(O)(aryl), S(O)₂(alkyl), S(O)₂(aryl), N₃, NH₂, NH(alkyl), N(alkyl)₂, NH(aryl), NH(aryl)₂, NO₂, CHO, C(O)(alkyl), C(O)(aryl), COOH, COO(alkyl) COO(aryl), C(O)NH(alkyl), C(O)NH(aryl), C(O)N(alkyl)₂, C(O)N(aryl)₂, P(O)OH(alkyl), P(O)OH(aryl), P(O)(—O-alkyl)₂, P(O)(—O-aryl)₂, PO₃H₂, SO₃H, alkyl, substituted alkyl, alkenyl, and substituted alkenyl;
R₂ and R₈ or R₇ and R₈ taken together may form a cyclic structure;
R₅ and R₉ or R₉ and R₁₀ taken together may form a cyclic structure;
R₃ and R₇ or R₄ and R₉ taken together may form a cyclic structure;
R₁ and R₂ or R₅ and R₆ taken together may form a cyclic structure;
X is selected from the group consisting of CR₁₄R₁₅, a heteroatom, NR₁₄, SO₂, P(O)OH, P(O)OR₁₄, SiR₁₄R₁₅, and GeR₁₄R₁₅, where R₁₄ and R₁₅ are alkyl or aryl;
Z is selected from the group consisting of O(alkyl), O(aryl), S(aryl), S(O)(alkyl), S(O)(aryl), S(O)₂(alkyl), S(O)₂(aryl), S(O)₂(—O-alkyl), S(O)₂(—O-aryl), S(O)₂NH(alkyl), S(O)₂NH(aryl), S(O)₂N(alkyl)₂, S(O)₂N(aryl)₂, S(O)₂N(alkyl)(aryl), C(O)O(alkyl), C(O)O(aryl), C(O)(alkyl), C(O)(aryl), P(O)OH(—NH-alkyl), P(O)OH(—O-alkyl), P(O)OH(—NH-aryl), P(O)OH(—O-aryl), P(O)(—O-alkyl)₂, P(O)(—NH-alkyl)₂, P(O)OH(—N(alkyl)₂), P(O)OH(—N(aryl)₂), P(O)(—N(aryl)₂)₂, P(O)(—N(alkyl)₂)(—N(alkyl)₂), P(O)(—O-aryl)₂, P(O)(—NH-aryl)₂, P(O)(—O-alkyl)(-O-aryl), P(O)(—NH-alkyl)(-O-aryl), P(O)(—O-alkyl)(-NH-aryl), P(O)(—NH-alkyl)(-NH-aryl), C(O)OH, C(O)NH(alkyl), C(O)NH(aryl), CON(alkyl)₂, CON(aryl)₂, and any other substituent which is not Cl, NH₂ or NO₂ and which induces a neighboring group effect via steric, ionic or bonding interactions with an adjacent carboxyl group involved in spirolactone formation resulting in a shift of an equilibrium between zwitterionic form and spirolactone form towards the spirolactone form as indicated by an increased D₅₀ value of a 4′-isomer compared to the D₅₀ value of a reference isomer, wherein said D₅₀ value represents the dielectric constant at which an absorbance of a dye sample in a mixture of 1,4-dioxane and water is halved.

2. The fluorescent dye according to claim 1, having one of the following structural formulae B-E 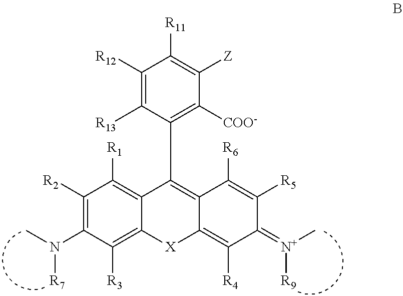
wherein the cyclic structure formed by R₇ and R₈ and/or by R₉ and R₁₀ taken together represents a non-aromatic heterocycle; 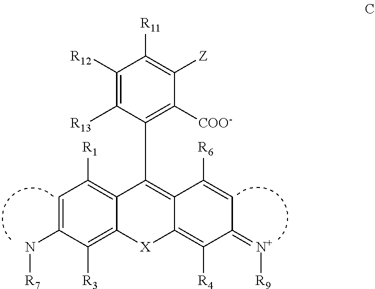
wherein the cyclic structure formed by R₂ and R₈ and/or by R₅ and R₁₀ taken together represents an aromatic or non-aromatic 5 or 6 atom membered heterocyclic structure; 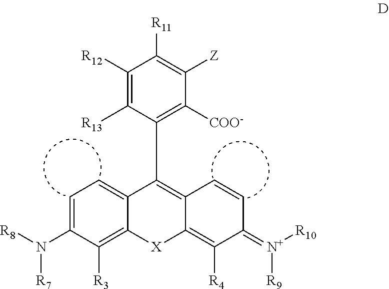
wherein the cyclic structure formed by R₁ and R₂ and/or by R₅ and R₆ taken together represents an aromatic or non-aromatic 5 or 6 atom membered heterocyclic structure; 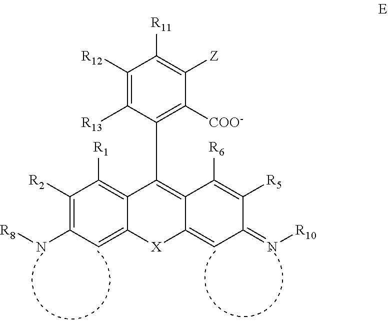
wherein the cyclic structure formed by R₃ and R₇ and/or by R₄ and R₉ taken together represents an aromatic or non-aromatic 5 or 6 atom membered heterocyclic structure.

3. The fluorescent dye according to claim 1, having one of the following structural formulae 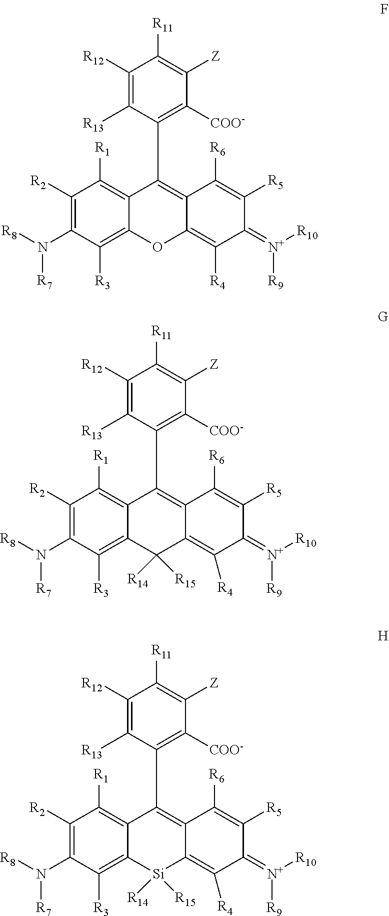
wherein
R₁, R₂, R₃, R₄, R₅, R₆, R₇, R₈, R₉, R₁₀, R₁₁, R₁₂ and R₁₃ are independently selected from H, halogen, D, CN, OH, O(alkyl), O(aryl), SH, S(alkyl), S(aryl), S(O)(alkyl), S(O)(aryl), S(O)₂(alkyl), S(O)₂(aryl), N₃, NH₂, NH(alkyl), N(alkyl)₂, NH(aryl), NH(aryl)₂, NO₂, CHO, C(O)(alkyl), C(O)(aryl), COOH, COO(alkyl) COO(aryl), C(O)NH(alkyl), C(O)NH(aryl), C(O)N(alkyl)₂, C(O)N(aryl)₂, P(O)OH(alkyl), P(O)OH(aryl), P(O)(—O-alkyl)₂, P(O)(—O-aryl)₂, PO₃H₂, SO₃H, alkyl, substituted alkyl, alkenyl, and substituted alkenyl;
R₂ and R₈ or R₇ and R₈ taken together may form a cyclic structure;
R₅ and R₁₀ or R₉ and R₁₀ taken together may form a cyclic structure;
R₃ and R₇ or R₄ and R₉ taken together may form a cyclic structure;
R₁ and R₂ or R₅ and R₆ taken together may form a cyclic structure;
Z is selected from the group consisting of O(alkyl), O(aryl), S(aryl), S(O)(alkyl), S(O)(aryl), S(O)₂(alkyl), S(O)₂(aryl), S(O)₂(—O-alkyl), S(O)₂(—O-aryl), S(O)₂NH(alkyl), S(O)₂NH(aryl), S(O)₂N(alkyl)₂, S(O)₂N(aryl)₂, S(O)₂N(alkyl)(aryl), C(O)O(alkyl), C(O)O(aryl), C(O)(alkyl), C(O)(aryl), P(O)OH(—NH-alkyl), P(O)OH(—O-alkyl), P(O)OH(—NH-aryl), P(O)OH(—O-aryl), P(O)(—O-alkyl)₂, P(O)(—NH-alkyl)₂, P(O)OH(—N(alkyl)₂), P(O)OH(—N(aryl)₂), P(O)(—N(aryl)₂)₂, P(O)(—N(alkyl)₂)(—N(alkyl)₂), P(O)(—O-aryl)₂, P(O)(—NH-aryl)₂, P(O)(—O-alkyl)(-O-aryl), P(O)(—NH-alkyl)(-O-aryl), P(O)(—O-alkyl)(-NH-aryl), P(O)(—NH-alkyl)(-NH-aryl), C(O)OH, C(O)NH(alkyl), C(O)NH(aryl), CON(alkyl)₂, CON(aryl)₂, and any other substituent which induces the neighboring group effect as defined in claim 1.

4. The fluorescent dye according to claim 1, having one of the following structural formulae: 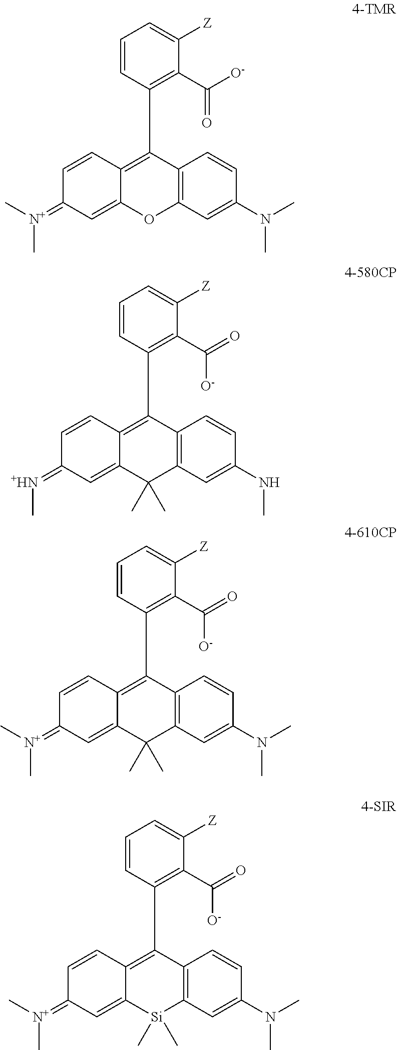
wherein
Z is selected from the group consisting of O(alkyl), O(aryl), S(aryl), S(O)(alkyl), S(O)(aryl), S(O)₂(alkyl), S(O)₂(aryl), S(O)₂(—O-alkyl), S(O)₂(—O-aryl), S(O)₂NH(alkyl), S(O)₂NH(aryl), S(O)₂N(alkyl)₂, S(O)₂N(aryl)₂, S(O)₂N(alkyl)(aryl), C(O)O(alkyl), C(O)O(aryl), C(O)(alkyl), C(O)(aryl), P(O)OH(—NH-alkyl), P(O)OH(—O-alkyl), P(O)OH(—NH-aryl), P(O)OH(—O-aryl), P(O)(—O-alkyl)₂, P(O)(—NH-alkyl)₂, P(O)OH(—N(alkyl)₂), P(O)OH(—N(aryl)₂), P(O)(—N(aryl)₂)₂, P(O)(—N(alkyl)₂)(—N(alkyl)₂), P(O)(—O-aryl)₂, P(O)(—NH-aryl)₂, P(O)(—O-alkyl)(-O-aryl), P(O)(—NH-alkyl)(-O-aryl), P(O)(—O-alkyl)(-NH-aryl), P(O)(—NH-alkyl)(-NH-aryl), C(O)OH, C(O)NH(alkyl), C(O)NH(aryl), CON(alkyl)₂, CON(aryl)₂, and any other substituent which induces a neighboring group effect as defined in claim 1.

5. The fluorescent dye according to claim 1, wherein Z is selected from the group consisting of C(O)OH, C(O)NH(alkyl), C(O)NH(aryl), CON(alkyl)₂, CON(aryl)₂, C(O)O(alkyl), and C(O)O(aryl).

6. A rhodamine 4′-isomer derivative comprising a rhodamine 4′-isomer having one of the following general structural formulae A-H: 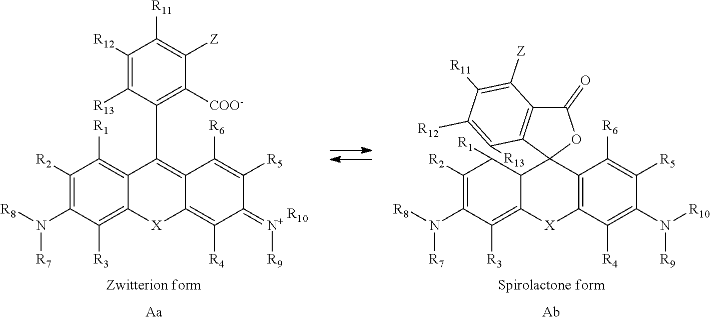
wherein
R₁, R₂, R₃, R₄, R₅, R₆, R₇, R₈, R₉, R₁₀, R₁₁, R₁₂ and R₁₃ are independently selected from H, halogen, D, CN, OH, O(alkyl), O(aryl), SH, S(alkyl), S(aryl), S(O)(alkyl), S(O)(aryl), S(O)₂(alkyl), S(O)₂(aryl), N₃, NH₂, NH(alkyl), N(alkyl)₂, NH(aryl), NH(aryl)₂, NO₂, CHO, C(O)(alkyl), C(O)(aryl), COOH, COO(alkyl) COO(aryl), C(O)NH(alkyl), C(O)NH(aryl), C(O)N(alkyl)₂, C(O)N(aryl)₂, P(O)OH(alkyl), P(O)OH(aryl), P(O)(—O-alkyl)₂, P(O)(—O-aryl)₂, PO₃H₂, SO₃H, alkyl, substituted alkyl, alkenyl, and substituted alkenyl;
R₂ and R₈ or R₇ and R₈ taken together may form a cyclic structure;
R₅ and R₉ or R₉ and R₁₀ taken together may form a cyclic structure;
R₃ and R₇ or R₄ and R₉ taken together may form a cyclic structure;
R₁ and R₂ or R₅ and R₆ taken together may form a cyclic structure;
X is selected from the group consisting of CR₁₄R₁₅, O, S, NR₁₄, SO₂, P(O)OH, P(O)OR₁₄, SiR₁₄R₁₅, and GeR₁₄R₁₅, where R₁₄ and R₁₅ are alkyl or aryl; 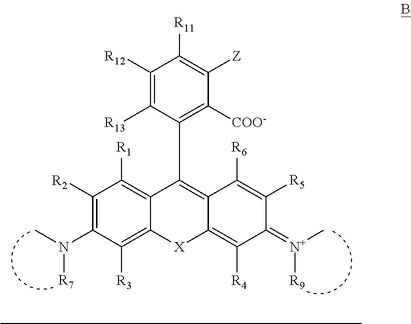
wherein the cyclic structure formed by R₇ and R₈ and/or by R₉ and R₁₀ taken together represents a non-aromatic heterocyle; 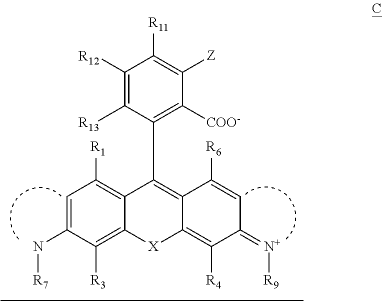
wherein the cyclic structure formed by R₇ and R₈ and/or by R₉ and R₁₀ taken together represents an aromatic or non-aromatic 5 or 6 atom membered heterocyclic structure; 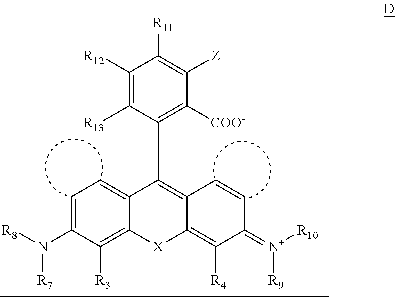
wherein the cyclic structure formed by R₁ and R₂ and/or by R₅ and R₆ taken together represents an aromatic or non-aromatic 5 or 6 atom membered heterocyclic structure; 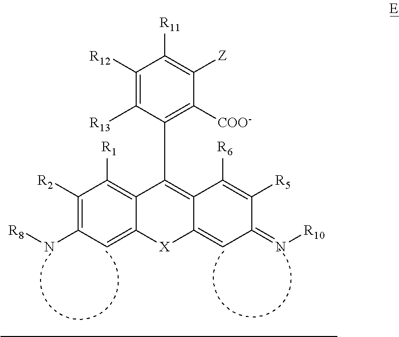
wherein the cyclic structure formed by R₃ and R₇ and/or by R₄ and R₉ taken together represents an aromatic or non-aromatic 5 or 6 atom membered heterocyclic structure; 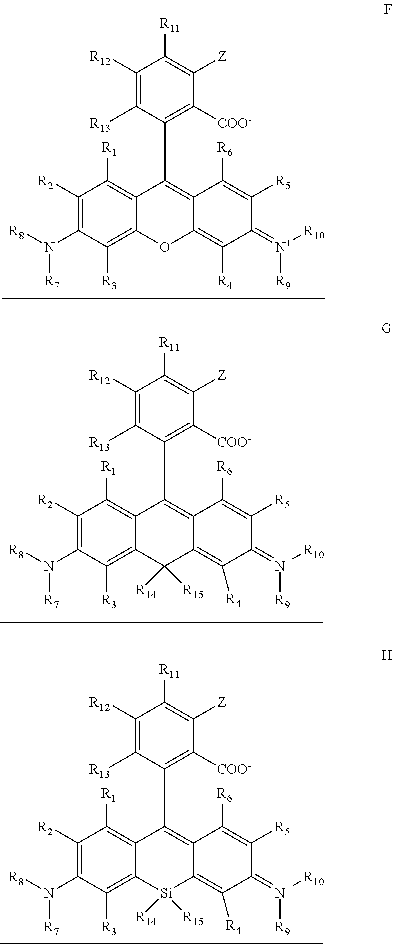
wherein
R₁, R₂, R₃, R₄, R₅, R₆, R₇, R₈, R₉, R₁₀, R₁₁, R₁₂ and R₁₃ are independently selected from H, halogen, D, CN, OH, O(alkyl), O(aryl), SH, S(alkyl), S(aryl), S(O)(alkyl), S(O)(aryl), S(O)₂(alkyl), S(O)₂(aryl), N₃, NH₂, NH(alkyl), N(alkyl)₂, NH(aryl), NH(aryl)₂, NO₂, CHO, C(O)(alkyl), C(O)(aryl), COOH, COO(alkyl) COO(aryl), C(O)NH(alkyl), C(O)NH(aryl), C(O)N(alkyl)₂, C(O)N(aryl)₂, P(O)OH(alkyl), P(O)OH(aryl), P(O)(—O-alkyl)₂, P(O)(—O-aryl)₂, PO₃H₂, SO₃H, alkyl, substituted alkyl, alkenyl, and substituted alkenyl;
R₂ and R₈ or R₇ and R₈ taken together may form a cyclic structure;
R₅ and R₁₀ or R₉ and R₁₀ taken together may form a cyclic structure;
R₃ and R₇ or R₄ and R₉ taken together may form a cyclic structure;
R₁ and R₂ or R₅ and R₆ taken together may form a cyclic structure;
wherein Z is selected from the group consisting of O(alkyl), O(aryl), S(aryl), S(O)(alkyl), S(O)(aryl), S(O)₂(alkyl), S(O)₂(aryl), S(O)₂(—O-alkyl), S(O)₂(—O-aryl), S(O)₂NH(alkyl), S(O)₂NH(aryl), S(O)₂N(alkyl)₂, S(O)₂N(aryl)₂, S(O)₂N(alkyl)(aryl), C(O)O(alkyl), C(O)O(aryl), C(O)(alkyl), C(O)(aryl), P(O)OH(—NH-alkyl), P(O)OH(—O-alkyl), P(O)OH(—NH-aryl), P(O)OH(—O-aryl), P(O)(—O-alkyl)₂, P(O)(—NH-alkyl)₂, P(O)OH(—N(alkyl)₂), P(O)OH(—N(aryl)₂), P(O)(—N(aryl)₂)₂, P(O)(—N(alkyl)₂)(-N(alkyl)₂), P(O)(—O-aryl)₂, P(O)(—NH-aryl)₂, P(O)(—O-alkyl)(-O-aryl), P(O)(—NH-alkyl)(-O-aryl), P(O)(—O-alkyl)(-NH-aryl), P(O)(—NH-alkyl)(-NH-aryl), C(O)OH, C(O)NH(alkyl), C(O)NH(aryl), CON(alkyl)₂, CON(aryl)₂, and any other substituent which is not Cl, NH₂ or NO₂ and which induces a neighboring group effect via steric, ionic or bonding interactions as indicated by an increased D₅₀ value of a 4′-isomer derivative compared to the D₅₀ value of a reference isomer derivative, wherein said D₅₀ value represents a dielectric constant at which an absorbance of a dye sample in a mixture of 1,4-dioxane and water is halved,
coupled to at least one reactive group or ligand which is capable to interact with or bind to other molecules, wherein said reactive group or ligand is coupled to the rhodamine 4′-isomer fluorophore either directly or via a linker.

7. The rhodamine 4′-isomer derivative according to claim 6, wherein Z is selected from the group consisting of C(O)OH, C(O)NH(alkyl), C(O)NH(aryl), CON(alkyl)₂, CON(aryl)₂, C(O)O(alkyl) and C(O)O(aryl).

8. The rhodamine 4′-isomer derivative according to claim 6, wherein the linker comprises a straight or branched alkyl chain with 1-21 C atoms optionally substituted by one or more functional groups, optionally including heteroatoms and/or aromatic groups

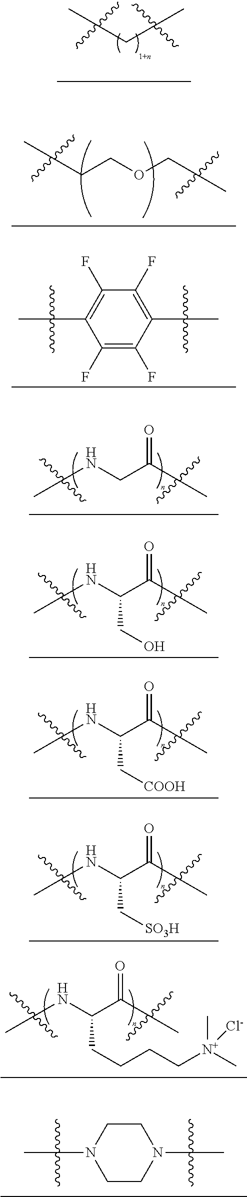

9. The rhodamine 4′-isomer derivative according to claim 6, wherein the reactive group is selected from the group consisting of an activated ester, an amine, a thiol, an azide, an ethyne, a maleimide, a tetrazine, N-hydroxysuccinimide and e an alcohol group, or wherein the ligand is a ligand which binds specifically to a protein, peptide, nucleotide or nucleic acid, carbohydrate, iodoacetamide or which is capable to effect or participate in chelation of NH₄⁺ or metal ions.

10. The rhodamine 4′-isomer derivative according to claim 6, wherein the reactive group or ligand is coupled directly or via a linker to group Z.

11. The rhodamine 4′-isomer derivative according claim 10, wherein the reactive group or ligand is coupled to group Z by an amide bond formed between a carboxyl functional group or amine functional group provided by Z and an amine functional group or carboxyl functional group provided by the linker or ligand, or wherein the ligand is selected from the group consisting of benzylguanine, benzylcytosine, a primary alkyl chloride and trimethoprim.

12. A conjugate comprising a rhodamine 4′-isomer according to claim 1 coupled to or associated with a molecule which is selected from the group consisting of a peptide, a protein, a nucleotide, a nucleic acid sequence, a lipid, a carbohydrate, an organic polyphosphate, an inorganic polyphosphate, a pharmaceutical drug or its metabolite, NH₄⁺, a metal ion, and a complex thereof.

13. A method of forming a conjugate, said method comprising conjugating the fluorescent dye according to claim 1 with an analyte or molecule of interest, which is selected from the group consisting of a peptide, a protein, a nucleotide, a nucleic acid sequence, a lipid, a carbohydrate, an organic polyphosphate, an inorganic polyphosphate, a pharmaceutical drug or its metabolite, a toxin, a reactive oxygen species (ROS), NH₄⁺, Li⁺, Na⁺, K⁺, Cs⁺, Rb⁺, Cu⁺, Tl⁺, Hg⁺, Ag⁺, Au⁺, Ca²⁺, Ba²⁺, Sr²⁺, Be²⁺, Zn²⁺, Ni²⁺, Cu²⁺, Mg²⁺, Co²⁺, Fe²⁺, Mn²⁺, Pt²⁺, Cd²⁺, Hg²⁺, Sn²⁺, Pb²⁺, Au³⁺, Cr³⁺, Co³⁺, Fe³⁺, Al³⁺, Ga³⁺, In³⁺, Sc³⁺, Ti³⁺, Tl³⁺, V³⁺, Y³⁺, La³⁺, Pt⁴⁺, Pb⁴⁺, Ce⁴⁺, Ge⁴⁺, Th⁴⁺, Zr⁴⁺, U⁴⁺, and a complex thereof.

14. The method according to claim 13, wherein the conjugating comprises formation of at least one covalent chemical bond or at least one molecular complex with a chemical entity or substance.

15. A method of forming a conjugate, said method comprising conjugating the rhodamine 4′-isomer derivative according to claim 6 with an analyte or molecule of interest, which is selected from the group consisting of a peptide, a protein, a nucleotide, a nucleic acid sequence, a lipid, a carbohydrate, an organic polyphosphate, an inorganic polyphosphate, a pharmaceutical drug or its metabolite, a toxin, a reactive oxygen species (ROS), NH₄⁺, Li⁺, Na⁺, K⁺, Cs⁺, Rb⁺, Cu⁺, Tl⁺, Hg⁺, Ag⁺, Au⁺, Ca²⁺, Ba²⁺, Sr²⁺, Be²⁺, Zn²⁺, Ni²⁺, Cu²⁺, Mg²⁺, Co²⁺, Fe²⁺, Mn²⁺, Pt²⁺, Cd²⁺, Hg²⁺, Sn²⁺, Pb²⁺, Au³⁺, Cr³⁺, Co³⁺, Fe³⁺, Al³⁺, Ga³⁺, In³⁺, Sc³⁺, Ti³⁺, Tl³⁺, V³⁺, Y³⁺, La³⁺, Pt⁴⁺, Pb⁴⁺, Ce⁴⁺, Ge⁴⁺, Th⁴⁺, Zr⁴⁺, U⁴⁺, and a complex thereof, wherein the ligand of the rhodamine 4′-isomer derivative which is capable to interact with or bind to other molecules is benzylguanine and the protein is SNAP-tag, or the ligand is benzylcytosine and the protein is CLIP-tag, or the ligand is a primary alkyl chloride and the protein is Halo-tag, or the ligand is trimethoprim and the protein is dihydrofolate reductase.

16. A method of penetrating a cell membrane, said method comprising contacting the cell membrane with the fluorescent dye according to claim 1 so that the fluorescent dye penetrates through the cell membrane of living and fixed cells in vivo or in vitro.

17. A detection method comprising adding the fluorescent dye according to claim 1 to a sample, optionally photoactivating the fluorescent dye and tracking and monitoring dynamic processes in the sample.

18. The detection method according to claim 17, wherein a rhodamine 4′-isomer derivative coupled to tubulin, DNA, RNA, lipid, or actin is used for cell cycle monitoring in living cells or living tissues in vivo or in vitro.

19. A treatment method comprising administering to living cells or a living organism the rhodamine 4′-isomer derivative according to claim 6 having singlet oxygen (¹O₂) quantum yield of at least 5%, for chromophore-assisted light inactivation (CALI) and photodynamic therapy (PDT).

20. A labeling method comprising using the fluorescent dye according to claim 1 as a label in microscopic, spectroscopic and other imaging techniques, in microfluidic devices, capillary electrophoresis, fluorescence activated cell sorting, DNA sequencing, sequence-specific genome labelling, analyte tracking techniques in vitro or in vivo.

21. The labeling method according to claim 20, wherein the analyte tracking techniques comprise at least one of the following sequences of steps:
interacting with or binding of the rhodamine 4′-isomer derivative to the analyte, which is a metal ion, resulting in metal ion chelation and in a corresponding change in fluorescence properties of the rhodamine 4′-isomer derivative, and using this change in fluorescence properties for metal ion imaging in vitro or in vivo, in particular in living cells and in living organisms including humans, mammals, birds, fish, hemichordates, molluscs, tunicates, cnidarians, cephalochordates, flatworms, nematodes, annelids, tardigrades, reptiles, arthropods, echinoderms, chaetognathas, rotifers, frogs, plants, sponges or fungi;
reacting of the rhodamine 4′-isomer derivative with reactive oxygen species (ROS) resulting in a corresponding change in fluorescence properties of the rhodamine 4′-isomer derivative, and using this change in fluorescence properties for ROS imaging in vitro or in vivo, in particular in living cells and in living organisms such as humans, mammals, birds, fish, hemichordates, molluscs, tunicates, cnidarians, cephalochordates, flatworms, nematodes, annelids, tardigrades, reptiles, arthropods, echinoderms, chaetognathas, rotifers, frogs, plants, sponges or fungi;
selectively interacting or reacting of the rhodamine 4′-isomer derivative with an enzyme, resulting in a corresponding change in fluorescence properties of the rhodamine 4′-isomer derivative, and using this change in fluorescence properties for detection, quantification and imaging of enzymatic activity in vitro and in vivo;
selectively interacting or reacting of the rhodamine 4′-isomer derivative with a lipid, organic and inorganic polyphosphate, protein, carbohydrate, metabolites, DNA or RNA, resulting in a corresponding change in fluorescence properties, and using this change in fluorescence properties for lipid, polyphosphate, protein, carbohydrate, metabolite, DNA or RNA imaging in vitro or in vivo;
interacting or reacting of rhodamine 4′-isomers derivatives coupled to any drug or drug candidate with a target molecule or target site in a cell or tissue, resulting in a corresponding change in fluorescence properties, and using this change in fluorescence properties for drug-target interaction monitoring using fluorescence imaging or NMR in vitro or in vivo;
interacting or reacting of a rhodamine 4′-isomer derivative with oxygen, fluoride or glucose through non-covalent complex or covalent bond formation, resulting in a corresponding change in fluorescence properties of the rhodamine 4′-isomer derivative, and using this change in fluorescence properties for oxygen, fluoride or glucose sensing in vitro or in vivo, in particular in living cells and in living organisms, including humans, mammals, birds, fish, hemichordates, molluscs, tunicates, cnidarians, cephalochordates, flatworms, nematodes, annelids, tardigrades, reptiles, arthropods, echinoderms, chaetognathas, rotifers, frogs, plants, sponges or fungi.

22. The labeling method according to claim 20, wherein the imaging techniques comprise stimulated emission depletion microscopy [STED], single molecule spectroscopy, single molecule switching (SMS) “nanoscopy” (diffraction unlimited optical resolution by using switching of the fluorescence of the single molecules, such as single molecule localization microscopy [SMLM], structured illumination microscopy (SIM), light-sheet microscopy, photoactivation localization microscopy [PALM, PALMIRA, fPALM], stochastic optical reconstruction microscopy [STORM]), fluorescence correlation spectroscopy [FCS] or fluorescence anisotropy spectroscopy, fluorescence recovery after photobleaching [FRAP], fluorescence lifetime imaging [FLIM], ground state depletion with individual molecular return [GSDIM], and fluorescence resonant energy transfer [FRET], correlative fluorescence—electron microscopy, correlative fluorescence—cryo-electron microscopy, microscale thermophoresis, fluorescence in situ hybridization (FISH), nuclear magnetic resonance spectroscopy.

23. The fluorescent dye according to claim 1, wherein the heteroatom of X is O or S.

24. The fluorescent dye according to claim 2, wherein the non-aromatic heterocycle of formulae B and the aromatic or non-aromatic 5 or 6 atom membered heterocyclic structure of formulae C-E are selected from the group consisting of azetidine, pyrrolidine, piperidine, azepane and azecane.
